(19) United States
(12) Patent Application Publication
(10) Pub. No.: US 2023/0256090 A1
(43) Pub. Date: Aug. 17, 2023

(54) ADJUVANTS

(71) Applicant: GLAXOSMITHKLINE BIOLOGICALS SA, Rixensart (BE)

(72) Inventor: Emmanuel Jules HANON, Rixensart (BE)

(73) Assignee: GLAXOSMITHKLINE BIOLOGICALS SA, Rixensart (BE)

(21) Appl. No.: 18/011,643

(22) PCT Filed: Jun. 25, 2021
(86) PCT No.: PCT/EP2021/067472

§ 371 Date: Dec. 20, 2022

Related U.S. Application Data
(60) Provisional application No. 63/045,375, filed on Jun. 29, 2020.
(60) Provisional application No. 63/139,896, filed on Jan. 21, 2021.

(51) Int. Cl. A61K 39/39; A61P 37/04

(52) U.S. Cl. CPC A61K 39/39; A61P 37/04; A61K 2039/55566

(57) ABSTRACT

The present invention relates to immunisation using carrier-formulated mRNA in conjunction with squalene emulsion adjuvants, and to related aspects.

### TECHNICAL FIELD

The present invention relates to carrier-formulated mRNA immunisation in conjunction with squalene emulsion adjuvants, and to related aspects.

### BACKGROUND ART

Messenger RNA (mRNA) is a single-stranded RNA molecule that corresponds to the genetic sequence of a gene and is read by ribosomes in the process of producing a protein. mRNA based vaccines provide an alternative vaccination approach to traditional strategies involving live attenuated/inactivated pathogens or subunit vaccines (Zhang, 2019). mRNA vaccines may utilise non-replicating mRNA or self-replicating RNA (also referred to as self-replicating or self-amplifying mRNA (‘SAM’)). Non-replicating mRNA-based vaccines typically encode an antigen of interest and contain 5′ and 3′ untranslated regions (UTRs), a 5′ cap and a poly(A) tail; whereas self-replicating RNAs also encode viral replication machinery that enables intracellular RNA amplification (Pardi, 2018).

Coronavirus disease 2019 (COVID-19) is an infectious disease caused by severe acute respiratory syndrome coronavirus 2 (SARS-CoV-2). The disease was first identified in late 2019 and has spread globally. The World Health Organization (WHO) declared the 2019-2020 coronavirus outbreak a Public Health Emergency of International Concern (PHEIC) on 30 Jan. 2020 and a pandemic on 11 Mar. 2020. The time from exposure to onset of symptoms is typically around five days but may range from two to fourteen days. While the majority of cases result in mild symptoms, some progress to viral pneumonia and multi-organ failure. As of 16 Jun. 2020, more than 7.89 million cases have been reported across 216 countries and territories, resulting in more than 433,000 deaths (WHO, 16 Jun. 2020). Management involves treatment of symptoms, supportive care, isolation and experimental measures.

mRNA-1273 is a vaccine against the novel coronavirus. mRNA-1273 encodes a prefusion stabilised form of the Spike (S) protein from SARS-CoV-2. Interim Phase 1 data showed mRNA-1273 elicited neutralizing antibody titre levels in all eight initial participants across the 25 ug and 100 ug dose cohorts, reaching or exceeding neutralizing antibody titres generally seen in convalescent sera. (Moderna Press Release, 18 May 2020)

Oil-in-water emulsion adjuvants containing squalene have featured in licensed pandemic and prepandemic influenza vaccines. ‘AS03’ (WO2006/100109; Garçon, 2012; Cohet, 2019) includes squalene, alpha-tocopherol and polysorbate 80. An adult human dose of AS03_{A} contains 10.69 mg squalene, 11.86 mg alpha-tocopherol and 4.86 mg polysorbate 80 (Fox, 2009; Morel, 2011). Certain reduced does of AS03 have also been described (WO2008/043774), including AS03_{B} (½ dose), AS03c (¼ dose) and AS03_{D} (⅛ dose) (Carmona Martinez, 2014). AS03 and MF59 (a submicron oil-in-water emulsion of squalene, polysorbate 80 and sorbitan trioleate) adjuvants have been shown to augment the immune responses to 2 doses of an inactivated H7N9 influenza vaccine, with the tocopherol containing AS03-adjuvanted formulations inducing the highest titers (Jackson, 2015).

Stable emulsions (SE) have also been described which contain squalene, phospholipid, poloxamer 188 (Pluronic F68) and glycerol in ammonium phosphate buffer (Carter, 2016). The SE have sometimes been described as containing low levels of alpha-tocopherol as an antioxidant (Sun, 2016).

There remains a need for the provision of further immunisation approaches that may have benefits such as reduced raw material need and/or reduced unsolicited effects (e.g. reduced reactogenicity), particularly while maintaining an adequate or even improved immune response.

### SUMMARY OF THE INVENTION

The inventors have found that squalene emulsion adjuvants may be of benefit in conjunction with carrier-formulated mRNA encoding an antigen.

The invention therefore provides a method of eliciting an immune response in a subject, the method comprising administering to the subject (i) carrier-formulated mRNA wherein the mRNA encodes an antigen, and (ii) a squalene emulsion adjuvant. Further provided is a method of adjuvanting the immune response of a subject to an antigen expressed following administration of carrier-formulated mRNA wherein the mRNA encodes an antigen, the method comprising administering to the subject a squalene emulsion adjuvant.

The invention also provides a squalene emulsion adjuvant for use in eliciting an immune response in a subject by administration with carrier-formulated mRNA wherein the mRNA encodes an antigen. Also provided is carrier-formulated mRNA wherein the mRNA encodes an antigen, for use in eliciting an immune response in a subject by administration with a squalene emulsion adjuvant.

The invention also provides the use of a squalene emulsion adjuvant in the manufacture of a medicament for use in eliciting an immune response in a subject by administration with carrier-formulated mRNA wherein the mRNA encodes an antigen. Also provided is the use of carrier-formulated mRNA, wherein the mRNA encodes an antigen, in the manufacture of a medicament for use in eliciting an immune response in a subject by administration with a squalene emulsion adjuvant.

The invention also provides an immunogenic composition comprising (i) carrier-formulated mRNA wherein the mRNA encodes an antigen, and (ii) a squalene emulsion adjuvant. Also provided is a kit comprising: (i) a first container comprising carrier-formulated mRNA wherein the mRNA encodes an antigen; and (ii) a second container comprising a squalene emulsion adjuvant. Additionally provided is a kit comprising: (i) a first container comprising carrier-formulated mRNA wherein the mRNA encodes an antigen; (ii) a second container comprising a squalene emulsion adjuvant, and (iii) instructions for combining the carrier-formulated mRNA (such as a single dose of the carrier-formulated mRNA) with the squalene emulsion adjuvant (such as a single dose of the squalene emulsion adjuvant) to produce an immunogenic composition prior to administration of a single dose of the immunogenic composition to a subject.

The invention also provides the use of (i) carrier-formulated mRNA wherein the mRNA encodes an antigen, and (ii) a squalene emulsion adjuvant, in the manufacture of a medicament.

### BRIEF DESCRIPTION OF THE SEQUENCES

SEQ ID NO: 1: SARS-CoV-2 S protein
SEQ ID NO: 2: SARS-CoV-2 S protein ectodomain
SEQ ID NO: 3: SARS-CoV-2 S protein receptor binding domain
SEQ ID NO: 4: Pre-fusion stabilised SARS-CoV-2 S protein ectodomain
SEQ ID NO: 5: Polynucleotide sequence of HSV-2 gE/gl insert
SEQ ID NO: 6: Polynucleotide sequence of parent SAM pTC83R_P989 plasmid
SEQ ID NO: 7: Polynucleotide sequence of SAM gE_P317R_IRES_gl transcript
SEQ ID NO: 8: SAM pTC83R_P989 transcript theoretical RNA sequence
SEQ ID NO: 9: Polypeptide sequence of HSV-2 gE P317R
SEQ ID NO: 10: Polypeptide sequence of HSV-2 gl
SEQ ID NO: 11: Polynucleotide sequence of mRNA encoding HSV-2 gE
SEQ ID NO: 12: Polynucleotide sequence of chemically modified mRNA encoding HSV-2 gE
SEQ ID NO: 13: Polypeptide sequence of encoded HSV-2 gE

### BRIEF DESCRIPTION OF THE FIGURES

FIG. 1: Evaluation of total anti-HSV-2 gE IgG antibody response measured in serum samples collected after one or two 0.1 ug doses of LNP/SAM-gE_P317R/gl vaccine co-administered at different timings and with different doses of AS03 adjuvant.

FIG. 2: Evaluation of total anti-HSV-2 gE IgG antibody response measured in serum samples collected after one or two 0.01 ug doses of LNP/SAM-gE_P317R/gl vaccine co-administered at different timings and with different doses of AS03 adjuvant.

FIG. 3: Evaluation of total anti-HSV-2 gl IgG antibody response measured in serum samples collected after one or two 0.1 ug doses of LNP/SAM-gE_P317R/gl vaccine co-administered at different timings and with different doses of AS03 adjuvant.

FIG. 4: Evaluation of total anti-HSV-2 gl IgG antibody response measured in serum samples collected after one or two 0.01 ug doses of LNP/SAM-gE_P317R/gl vaccine co-administered at different timings and with different doses of AS03 adjuvant.

FIG. 5: Inhibition of hlgG Fc binding activity by HSV-2 gE/gl protein (ED50) 22 days after the second 0.1 ug dose of LNP/SAM-gE_P317R/gl vaccine co-administered at different timings and with different doses of AS03 adjuvant.

FIG. 6: Inhibition of hlgG Fc binding activity by HSV-2 gE/gl protein (ED50) 22 days after the second 0.01 ug dose of LNP/SAM-gE_P317R/gl vaccine co-administered at different timings and with different doses of AS03 adjuvant.

FIG. 7: Percentage of anti-HSV-2 gE or gl-specific CD4+ T cell responses induced in CB6F1 mice 22 days after second immunization with 0.1 ug LNP/SAM-gE_P317R/gl vaccine co-administered at different timings and with different doses of AS03 adjuvant.

FIG. 8: Percentage of anti-HSV-2 gE or gl-specific CD4+ T cell responses induced in CB6F1 mice 22 days after second immunization with 0.01 ug LNP/SAM-gE_P317R/gl vaccine co-administered at different timings and with different doses of AS03 adjuvant.

FIG. 9: Percentage of anti-HSV-2 gE or gl-specific CD8+ T cell responses induced in CB6F1 mice 22 days after second immunization with 0.1 ug LNP/SAM-gE_P317R/gl vaccine co-administered at different timings and with different doses of AS03 adjuvant.

FIG. 10: Percentage of anti-HSV-2 gE or gl-specific CD8+ T cell responses induced in CB6F1 mice 22 days after second immunization with 0.01 ug LNP/SAM-gE_P317R/gl vaccine co-administered at different timings and with different doses of AS03 adjuvant.

FIG. 11: Evaluation of total anti-HSV-2 gE IgG antibody response measured in serum samples collected after one or two 0.1 ug doses of LNP/SAM-gE_P317R/gl vaccine co-administered at different timings and with different doses of AddaVax™ adjuvant.

FIG. 12: Evaluation of total anti-HSV-2 gE IgG antibody response measured in serum samples collected after one or two 0.01 ug doses of LNP/SAM-gE_P317R/gl vaccine co-administered at different timings and with different doses of AddaVax™ adjuvant.

FIG. 13: Evaluation of total anti-HSV-2 gl IgG antibody response measured in serum samples collected after one or two 0.1 ug doses of LNP/SAM-gE_P317R/gl vaccine co-administered at different timings and with different doses of AddaVax™ adjuvant.

FIG. 14: Evaluation of total anti-HSV-2 gl IgG antibody response measured in serum samples collected after one or two 0.01 ug doses of LNP/SAM-gE_P317R/gl vaccine co-administered at different timings and with different doses of AddaVax™ adjuvant.

FIG. 15: Inhibition of hlgG Fc binding activity by HSV-2 gE/gl protein (ED50) 22 days after the second 0.1 ug dose of LNP/SAM-gE_P317R/gl vaccine co-administered at different timings and with different doses of AddaVax™ adjuvant.

FIG. 16: Inhibition of hlgG Fc binding activity by HSV-2 gE/gl protein (ED50) 22 days after the second 0.01 ug dose of LNP/SAM-gE_P317R/gl vaccine co-administered at different timings and with different doses of AddaVax™ adjuvant.

FIG. 17: Percentage of anti-HSV-2 gE or gl-specific CD4+ T cell responses induced in CB6F1 mice 22 days after second immunization with 0.1 ug LNP/SAM-gE_P317R/gl vaccine co-administered at different timings and with different doses of AddaVax™ adjuvant.

FIG. 18: Percentage of anti-HSV-2 gE or gl-specific CD4+ T cell responses induced in CB6F1 mice 22 days after second immunization with 0.01 ug LNP/SAM-gE_P317R/gl vaccine co-administered at different timings and with different doses of AddaVax™ adjuvant.

FIG. 19: Percentage of anti-HSV-2 gE or gl-specific CD8+ T cell responses induced in CB6F1 mice 22 days after second immunization with 0.1 ug LNP/SAM-gE_P317R/gl vaccine co-administered at different timings and with different doses of AddaVax™ adjuvant.

FIG. 20: Percentage of anti-HSV-2 gE or gl-specific CD8+ T cell responses induced in CB6F1 mice 22 days after second immunization with 0.01 ug LNP/SAM-gE_P317R/gl vaccine co-administered at different timings and with different doses of AddaVax™ adjuvant.

FIG. 21: Evaluation of total anti-HSV-2 gE IgG antibody response measured in serum samples collected after one or two 0.8 ug doses of LNP/mRNA (native) vaccine co-administered at different timings and with different doses of AS03 adjuvant.

FIG. 22: Evaluation of total anti-HSV-2 gE IgG antibody response measured in serum samples collected after one or two 0.8 ug doses of LNP/mRNA (N1mψ) vaccine co-administered at different timings and with different doses of AS03 adjuvant.

FIG. 23: Evaluation of total anti-HSV-1 gE/gl cross-reactive IgG antibody response measured in serum samples collected after one or two 0.8 ug doses of LNP/mRNA (native) vaccine co-administered at different timings and with different doses of AS03 adjuvant.

FIG. 24: Evaluation of total anti-HSV-1 gE/gl cross-reactive IgG antibody response measured in serum samples collected after one or two 0.8 ug doses of LNP/mRNA (N1mψ) vaccine co-administered at different timings and with different doses of AS03 adjuvant.

FIG. 25: Inhibition of hlgG Fc binding activity by HSV-2 gE/gl protein (ED50) 14PII for LNP/mRNA (native) vaccine co-administered at different timings and with different doses of AS03 adjuvant.

FIG. 26: Inhibition of hlgG Fc binding activity by HSV-2 gE/gl protein (ED50) 14PII for LNP/mRNA (N1mψ) vaccine co-administered at different timings and with different doses of AS03 adjuvant.

FIG. 27: Percentage of anti-HSV-2 gE or anti-HSV-1 gE cross-reactive CD4+ T cell responses induced in CB6F1 mice 14 days after second immunization with 0.8 ug LNP/mRNA (native) vaccine co-administered at different timings and with different doses of AS03 adjuvant.

FIG. 28: Percentage of anti-HSV-2 gE or anti-HSV-1 gE cross-reactive CD4+ T cell responses induced in CB6F1 mice 14 days after second immunization with 0.8 ug LNP/mRNA (N1mψ) vaccine co-administered at different timings and with different doses of AS03 adjuvant.

FIG. 29: Percentage of anti-HSV-2 gE or anti-HSV-1 gE cross-reactive CD8+ T cell responses induced in CB6F1 mice 14 days after second immunization with 0.8 ug LNP/mRNA (native) vaccine co-administered at different timings and with different doses of AS03 adjuvant.

FIG. 30: Percentage of anti-HSV-2 gE or anti-HSV-1 gE cross-reactive CD8+ T cell responses induced in CB6F1 mice 14 days after second immunization with 0.8 ug LNP/mRNA (N1mψ) vaccine co-administered at different timings and with different doses of AS03 adjuvant.

FIG. 31: Evaluation of total anti-HSV-2 gE IgG antibody response measured in serum samples collected after one or two 0.8 ug doses of LNP/mRNA (native) vaccine co-administered at different timings and with different doses of AddaVax™ adjuvant.

FIG. 32: Evaluation of total anti-HSV-2 gE IgG antibody response measured in serum samples collected after one or two 0.8 ug doses of LNP/mRNA (N1mψ) vaccine co-administered at different timings and with different doses of AddaVax™ adjuvant.

FIG. 33: Evaluation of total anti-HSV-1 gE/gl cross-reactive IgG antibody response measured in serum samples collected after one or two 0.8 ug doses of LNP/mRNA (native) vaccine co-administered at different timings and with different doses of AddaVax™ adjuvant.

FIG. 34: Evaluation of total anti-HSV-1 gE/gl cross-reactive IgG antibody response measured in serum samples collected after one or two 0.8 ug doses of LNP/mRNA (N1mψ) vaccine co-administered at different timings and with different doses of AddaVax™ adjuvant.

FIG. 35: Inhibition of hlgG Fc binding activity by HSV-2 gE/gl protein (ED50) 14PII for LNP/mRNA (native) vaccine co-administered at different timings and with different doses of AddaVax™ adjuvant.

FIG. 36: Inhibition of hlgG Fc binding activity by HSV-2 gE/gl protein (ED50) 14PII for LNP/mRNA (N1mψ) vaccine co-administered at different timings and with different doses of AddaVax™ adjuvant.

FIG. 37: Percentage of anti-HSV-2 gE or anti-HSV-1 gE cross-reactive CD4+ T cell responses induced in CB6F1 mice 14 days after second immunization with 0.8 ug LNP/mRNA (native) vaccine co-administered at different timings and with different doses of AddaVax™ adjuvant.

FIG. 38: Percentage of anti-HSV-2 gE or anti-HSV-1 gE cross-reactive CD4+ T cell responses induced in CB6F1 mice 14 days after second immunization with 0.8 ug LNP/mRNA (N1mψ) vaccine co-administered at different timings and with different doses of AddaVax™ adjuvant.

FIG. 39: Percentage of anti-HSV-2 gE or anti-HSV-1 gE cross-reactive CD8+ T cell responses induced in CB6F1 mice 14 days after second immunization with 0.8 ug LNP/mRNA (native) vaccine co-administered at different timings and with different doses of AddaVax™ adjuvant.

FIG. 40: Percentage of anti-HSV-2 gE or anti-HSV-1 gE cross-reactive CD8+ T cell responses induced in CB6F1 mice 14 days after second immunization with 0.8 ug LNP/mRNA (N1mψ) vaccine co-administered at different timings and with different doses of AddaVax™ adjuvant.

### DETAILED DESCRIPTION OF THE INVENTION

As mentioned previously, the inventors have found that squalene emulsion adjuvants may be of benefit in conjunction with carrier-formulated mRNA encoding an antigen.

### Squalene Emulsion Adjuvants

The term ‘squalene emulsion adjuvant’ as used herein refers to a squalene containing oil-in-water emulsion adjuvant. The term ‘tocopherol containing squalene emulsion adjuvant’ as used herein refers to a squalene and tocopherol containing oil-in-water emulsion adjuvant wherein the weight ratio of squalene to tocopherol is 20 or less (i.e. 20 weight units of squalene or less per weight unit of tocopherol or, alternatively phrased, at least 1 weight unit of tocopherol per 20 weight units of squalene). Tocopherol containing squalene emulsion adjuvants are therefore a subset of squalene emulsion adjuvants and are of particular interest in the present invention.

Squalene, is a branched, unsaturated terpenoid ([(CH₃)₂C[═CHCH₂CH₂C(CH₃)]₂═CHCH₂-]₂; C₃₀H₅₀; 2,6,10,15,19,23-hexamethyl-2,6,10,14,18,22-tetracosahexaene; CAS Registry Number 7683-64-9). Squalene is readily available from commercial sources or may be obtained by methods known in the art. Squalene shows good biocompatibility and is readily metabolised.

Squalene emulsion adjuvants will typically have a submicron droplet size. Droplet sizes below 200 nm are beneficial in that they can facilitate sterilisation by filtration. There is evidence that droplet sizes in the 80 to 200 nm range are of particular interest for potency, manufacturing consistency and stability reasons. (Klucker, 2012; Shah, 2014; Shah, 2015; Shah, 2019). Suitably the squalene emulsion adjuvant has an average droplet size of less than 1 um, especially less than 500 nm and in particular less than 200 nm. Suitably the squalene emulsion adjuvant has an average droplet size of at least 50 nm, especially at least 80 nm, in particular at least 100 nm, such as at least 120 nm. The squalene emulsion adjuvant may have an average droplet size of 50 to 200 nm, such as 80 to 200 nm, especially 120 to 180 nm, in particular 140 to 180 nm, such as about 160 nm.

Uniformity of droplet sizes is desirable. A polydispersity index (PdI) of greater than 0.7 indicates that the sample has a very broad size distribution and a reported value of 0 means that size variation is absent, although values smaller than 0.05 are rarely seen. Suitably the squalene emulsion adjuvant has a polydispersity of 0.5 or less, especially 0.3 or less, such as 0.2 or less.

The droplet size, as used herein, means the average diameter of oil droplets in an emulsion and can be determined in various ways e.g. using the techniques of dynamic light scattering and/or single-particle optical sensing, using an apparatus such as the Accusizer™ and Nicomp™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan). See Light Scattering from Polymer Solutions and Nanoparticle Dispersions Schartl, 2007. Dynamic light scattering (DLS) is the preferred method by which droplet size is determined. The preferred method for defining the average droplet diameter is a Z-average i.e. the intensity-weighted mean hydrodynamic size of the ensemble collection of droplets measured by DLS. The Z-average is derived from cumulants analysis of the measured correlation curve, wherein a single particle size (droplet diameter) is assumed and a single exponential fit is applied to the autocorrelation function. Thus, references herein to average droplet size should be taken as an intensity-weighted average, and ideally the Z-average. Polydispersity Index (PdI) values are easily provided by the same instrumentation which measures average diameter.

In order to maintain a stable submicron emulsion, one or more emulsifying agents (i.e. surfactants) are generally required. Surfactants can be classified by their ‘HLB’ (Griffin's hydrophile/lipophile balance), where a HLB in the range 1-10 generally means that the surfactant is more soluble in oil than in water, whereas a HLB in the range 10-20 means that the surfactant is more soluble in water than in oil. HLB values are readily available for many surfactants of interest or can be determined experimentally, e.g. polysorbate 80 has a HLB of 15.0 and TPGS has a HLB of 13 to 13.2. Sorbitan trioleate has a HLB of 1.8. When two or more surfactants are blended, the resulting HLB of the blend is typically calculated by the weighted average e.g. a 70/30 wt % mixture of polysorbate 80 and TPGS has a HLB of (15.0×0.70)+(13×0.30) i.e. 14.4. A 70/30 wt % mixture of polysorbate 80 and sorbitan trioleate has a HLB of (15.0×0.70)+(1.8×0.30) i.e. 11.04.

Surfactant(s) will typically be metabolisable (biodegradable) and biocompatible, being suitable for use as a pharmaceutical. The surfactant can include ionic (cationic, anionic or zwitterionic) and/or non-ionic surfactants. The use of only non-ionic surfactants is often desirable, for example due to their pH independence. The invention can thus use surfactants including, but not limited to:
the polyoxyethylene sorbitan ester surfactants (commonly referred to as the Tweens or polysorbates), such as polysorbate 20 and polysorbate 80, especially polysorbate 80;
copolymers of ethylene oxide (EO), propylene oxide (PO), and/or butylene oxide (BO), sold under the DOWFAX™, Pluronic™ (e.g. F68, F127 or L121 grades) or Synperonic™ tradenames, such as linear EO/PO block copolymers, for example poloxamer 407, poloxamer 401 and poloxamer 188;
octoxynols, which can vary in the number of repeating ethoxy (oxy-1,2-ethanediyl) groups, with octoxynol-9 (Triton X-100, or t-octylphenoxypolyethoxyethanol) being of particular interest;
(octylphenoxy)polyethoxyethanol (IGEPAL CA-630/NP-40);
phospholipids such as phosphatidylcholine (lecithin);
polyoxyethylene fatty ethers derived from lauryl, cetyl, stearyl and oleyl alcohols (known as Brij surfactants), such as polyoxyethylene 4 lauryl ether (Brij 30, Emulgen 104P), polyoxyethylene-9-lauryl ether and polyoxyethylene 12 cetyl/stearyl ether (Eumulgin™ B1, cetereth-12 or polyoxyethylene cetostearyl ether);
sorbitan esters (commonly known as the Spans), such as sorbitan trioleate (Span 85), sorbitan monooleate (Span 80) and sorbitan monolaurate (Span 20);
or tocopherol derivative surfactants, such as alpha-tocopherol-polyethylene glycol succinate (TPGS).

Many examples of pharmaceutically acceptable surfactants are known in the art e.g. see Handbook of Pharmaceutical Excipients 6th edition, 2009. Methods for selecting an optimising the choice of surfactant used in a squalene emulsion adjuvant are illustrated in Klucker, 2012.

In general, the surfactant component has a HLB between 10 and 18, such as between 12 and 17, in particular 13 to 16. This can be typically achieved using a single surfactant or, in some embodiments, using a mixture of surfactants. Surfactants of particular interest include: poloxamer 401, poloxamer 188, polysorbate 80, sorbitan trioleate, sorbitan monooleate and polyoxyethylene 12 cetyl/stearyl ether either alone, in combination with each other or in combination with other surfactants. Especially of interest are polysorbate 80, sorbitan trioleate, sorbitan monooleate and polyoxyethylene 12 cetyl/stearyl ether either alone, or in combination with each other. A particular surfactant of interest is polysorbate 80. A particular combination of surfactants of interest is polysorbate 80 and sorbitan trioleate. A further combination of surfactants of interest is sorbitan monooleate and polyoxyethylene cetostearyl ether.

In certain embodiments the squalene emulsion adjuvant comprises one surfactant, such as polysorbate 80. In some embodiments the squalene emulsion adjuvant comprises two surfactants, such as polysorbate 80 and sorbitan trioleate or sorbitan monooleate and polyoxyethylene cetostearyl ether. In other embodiments the squalene emulsion adjuvant comprises three or more surfactants, such as three surfactants.

The amount of squalene in a single dose, such as a human dose, of squalene emulsion adjuvant may be 50 mg or less, especially 40 mg or less, in particular 30 mg or less, such as 20 mg or less (for example 15 mg or less). The amount of squalene in a single dose, such as a human dose, of squalene emulsion adjuvant may be 0.5 mg or more, especially 1 mg or more, in particular 2 mg or more, such as 4 mg or more and desirably 8 mg or more. The amount of squalene in a single dose, such as a human dose, of squalene emulsion adjuvant may be 0.5 to 50 mg, especially 1 to 20 mg, in particular 2 to 15 mg, such as 5 to 15 mg. The amount of squalene in a single dose, such as a human dose, of squalene emulsion adjuvant may be 0.5 to 2 mg, 2 to 4 mg, 4 to 8 mg, 8 to 12 mg, 12 to 16 mg, 16 to 20 mg or 20 to 50 mg.

The amount of squalene in a single dose, such as a human dose, of squalene emulsion adjuvant may be 1.2 to 20 mg, in particular 1.2 to 15 mg. The amount of squalene in a single dose, such as a human dose, of squalene emulsion adjuvant may be 1.2 to 2 mg, 2 to 4 mg, 4 to 8 mg or 8 to 12.1 mg. For example, the amount of squalene in a single dose, such as a human dose, of squalene emulsion adjuvant may be 1.21 to 1.52 mg, 2.43 to 3.03 mg, 4.87 to 6.05 mg or 9.75 to 12.1 mg.

Typically the weight ratio of squalene to surfactant is 0.73 to 6.6, especially 1 to 5, in particular 1.5 to 4.5. The weight ratio of squalene to surfactant may be 1.5 to 3, especially 1.71 to 2.8, such as 2.2 or 2.4. The weight ratio of squalene to surfactant may be 2.5 to 3.5, especially 3 or 3.1. The weight ratio of squalene to surfactant may be 3 to 4.5, especially 4 or 4.3.

The amount of surfactant in a single dose, such as a human dose, of squalene emulsion adjuvant is typically at least 0.4 mg. Generally, the amount of surfactant in a single dose, such as a human dose, of squalene emulsion adjuvant is 18 mg or less. The amount of surfactant in a single dose, such as a human dose, of squalene emulsion adjuvant may be 0.4 to 9.5 mg, in particular 0.4 to 7 mg. The amount of surfactant in a single dose, such as a human dose, of squalene emulsion adjuvant may be 0.4 to 1 mg, 1 to 2 mg, 2 to 4 mg or 4 to 7 mg. For example, the amount of surfactant in a single dose, such as a human dose, of squalene emulsion adjuvant may be 0.54 to 0.71 mg, 1.08 to 1.42 mg, 2.16 to 2.84 mg or 4.32 to 5.68 mg.

The squalene emulsion adjuvant may contain one or more tocopherols. Any of the α, β, γ, δ, ε and/or ξ tocopherols can be used, but α-tocopherol (also referred to herein as alpha-tocopherol) is typically used. D-alpha-tocopherol and D/L-alpha-tocopherol can both be used. Tocopherols are readily available from commercial sources or may be obtained by methods known in the art. In some embodiments the squalene emulsion adjuvant does not contain tocopherol. In some embodiments the squalene emulsion adjuvant contains tocopherol (i.e. at least one tocopherol, suitably one tocopherol), especially alpha-tocopherol, in particular D/L-alpha-tocopherol.

Tocopherols have been used, in relatively small amounts, in squalene emulsion adjuvants as antioxidants. Desirably tocopherols are present a level where the weight ratio of squalene to tocopherol is 20 or less, such as 10 or less. Suitably the weight ratio of squalene to tocopherol is 0.1 or more. Typically the weight ratio of squalene to tocopherol is 0.1 to 10, especially 0.2 to 5, in particular 0.3 to 3, such as 0.4 to 2. Suitably, the weight ratio of squalene to tocopherol is 0.72 to 1.136, especially 0.8 to 1, in particular 0.85 to 0.95, such as 0.9. Alternatively, the weight ratio of squalene to tocopherol is 3.4 to 4.6, especially 3.6 to 4.4, in particular 3.8 to 4.2, such as 4.

The amount of tocopherol in a single dose, such as a human dose, of squalene emulsion adjuvant is typically at least 0.5 mg, especially at least 1.3 mg. Generally, the amount of tocopherol in a single dose, such as a human dose, of squalene emulsion adjuvant is 55 mg or less. The amount of tocopherol in a single dose, such as a human dose, of squalene emulsion adjuvant may be 1.3 to 22 mg, in particular 1.3 to 16.6 mg. The amount of tocopherol in a single dose, such as a human dose, of squalene emulsion adjuvant may be 1.3 to 2 mg, 2 to 4 mg, 4 to 8 mg or 8 to 13.6 mg. For example, the amount of tocopherol in a single dose, such as a human dose, of squalene emulsion adjuvant may be 1.33 to 1.69 mg, 2.66 to 3.39 mg, 5.32 to 6.77 mg or 10.65 to 13.53 mg.

In certain embodiments the squalene emulsion adjuvant may consist essentially of squalene, tocopherol (if present), surfactant and water. In addition to squalene, tocopherol, surfactant and water, squalene emulsion adjuvants may contain additional components as desired or required depending upon the intended final presentation and vaccination strategy, such as buffers and/or tonicity modifying agents, for example modified phosphate buffered saline (disodium phosphate, potassium biphosphate, sodium chloride and potassium chloride).

A squalene emulsion of interest in the present invention is known as ‘MF59’ (WO90/14837; Podda, 2003; Podda, 2001) and is a submicron oil-in-water emulsion of squalene, polysorbate 80 (also known as Tween 80™), and sorbitan trioleate (also known as Span 85™). It may also include citrate ions e.g. 10 mM sodium citrate buffer. The composition of the emulsion by volume can be about 5% squalene, about 0.5% polysorbate 80 and about 0.5% sorbitan trioleate. The adjuvant and its production are described in more detail in Vaccine Design: The Subunit and Adjuvant Approach (chapter 10), Vaccine Adjuvants: Preparation Methods and Research Protocols (chapter 12) and New Generation Vaccines (chapter 19). As described in O'Hagan, 2007, MF59 is typically manufactured on a commercial scale by dispersing sorbitan trioleate in the squalene, dispersing polysorbate 80 in an aqueous phase (e.g. citrate buffer), then mixing these two phases to form a coarse emulsion which is then microfluidised. The emulsion is typically prepared at double-strength (4.3% v/v squalene, 0.5% v/v polysorbate 80 and 0.5% v/v sorbitan trioleate) and is diluted 1:1 (by volume) with an antigen composition to provide a final adjuvanted vaccine composition. An adult human dose of MF59 contains 9.75 mg squalene, 1.17 mg polysorbate 80 and 1.17 mg sorbitan trioleate (O'Hagan, 2013). An adult human dose of MF59C.1, as used in the seasonal influenza vaccine Fluad™, contains 9.75 mg squalene, 1.175 mg polysorbate 80 and 1.175 mg sorbitan trioleate, 0.66 mg sodium citrate and 0.04 mg citric acid (O'Hagan, 2013) in 0.5 ml of water for injection (Fluad™ Summary of Product Characteristics).

A further squalene emulsion of interest in the present invention is known as ‘AF03’ (US2007/0014805; Klucker, 2012). AF03 includes squalene, sorbitan monooleate, polyoxyethylene cetostearyl ether and mannitol. AF03 is typically prepared by cooling a pre-heated water-in-oil emulsion until it crosses its emulsion phase inversion temperature, at which point it thermoreversibly converts into an oil-in-water emulsion. The mannitol, cetostearyl ether and a phosphate buffer are mixed in one container to form an aqueous phase, while the sorbitan ester and squalene are mixed in another container to form an oily component. The aqueous phase is added to the oily component and the mixture is then heated to approximately 60° C. and cooled to provide the final emulsion. The emulsion is typically initially prepared as a concentrate with a composition of 32.5% squalene, 4.8% sorbitan monooleate, 6.2% polyoxyethylene cetostearyl ether and 6% mannitol and 50.5% phosphate buffered saline. AF03 adjuvant contains 12.4 mg squalene, 1.9 mg sorbitan monooleate, 2.4 mg polyoxyethylene cetostearyl ether and 2.3 mg mannitol per 500 ul human adult dose (Humenza™ Summary of Product Characteristics).

Another squalene emulsion of interest in the present invention is known as ‘AS03’ (Garçon, 2012) and is typically prepared by mixing an oil mixture (consisting of squalene and alpha-tocopherol) with an aqueous phase (polysorbate 80 and buffer), followed by microfluidisation (WO2006/100109). AS03 is typically prepared at double-strength with the expectation of dilution by an aqueous antigen containing composition prior to administration. An adult human dose of AS03_{A} contains 10.69 mg squalene, 11.86 mg alpha-tocopherol and 4.86 mg polysorbate 80 (Morel, 2011; Fox, 2009). Certain reduced does of AS03 have also been described (WO2008/043774), including AS03_{B} (½ dose), AS03c (¼ dose) and AS03_{D}(⅛ dose) (Carmona Martinez, 2014).

As discussed above, high pressure homogenization (HPH or microfluidisation) and a phase inversion temperature method (PIT) may be applied to yield squalene emulsion adjuvants which demonstrate uniformly small droplet sizes and long-term stability. More recently, squalene based self-emulsifying adjuvant systems (SEAS) have been described. WO2015/140138 and WO2016/135154 describe the preparation of oil/surfactant compositions, which when diluted with an aqueous phase spontaneously form oil-in-water emulsions having small droplet particle sizes, such emulsions can be used as immunological adjuvants. An adult human dose of ‘SEA160’ emulsion may include 7.62 mg squalene, 2.01 mg polysorbate 80 and 2.01 mg sorbitan trioleate. (Shah, 2014; Shah, 2015; Shah, 2019)

International patent application PCT/US2020/15565 (published as WO2020/160080) and Lodaya, 2019 describe further squalene based self-emulsifying adjuvant systems (SEAS), specifically systems comprising a tocopherol in addition to squalene. ‘SEAS44’ contains 60% v/v squalene, 15% v/v alpha-tocopherol and 25% v/v polysorbate 80. The squalene/tocopherol/polysorbate mixture is intended to be diluted approximately 10-fold with an aqueous medium to form the final emulsion adjuvant. Consequently, an adult human dose of SEAS44 emulsion may include about 13 mg squalene, 3.6 mg alpha-tocopherol and 6.7 mg polysorbate 80.

Other squalene emulsion adjuvants have been described including:
SWE (Younis, 2018) comprising squalene 3.9% w/v, sorbitan trioleate 0.47% w/v, and polysorbate 80 (0.47% w/v) dispersed in 10 mM citrate buffer at pH 6.5. Consequently, an adult human dose of SWE may include about 9.75 mg squalene, 1.175 mg sorbitan trioleate and 1.175 mg polysorbate 80, similar to MF59.
SE (Carter, 2016; Sun, 2016) comprising squalene, phosphatidyl choline, poloxamer 188 and an ammonium phosphate buffered aqueous phase also containing glycerol. Sometimes SE has been described as containing small amounts of tocopherol. An adult human dose of SE may include about 8.6 mg squalene, 2.73 mg phosphatidyl choline and 0.125 mg poloxamer 188, optionally with 0.05 mg tocopherol.
CoVaccine (Hilgers, 2006; Hamid, 2011; Younis, 2019) comprises squalene, polysorbate 80 and sucrose fatty acid sulfate esters, typically with phosphate buffered saline. An adult human dose of CoVaccine may include about 40 mg squalene, 10 mg polysorbate 80 and 10 mg sucrose fatty acid sulfate esters.

The squalene emulsion adjuvant may be derived from MF59. Consequently, the squalene emulsion adjuvant may comprise squalene, polysorbate 80, sorbitan trioleate and water. The squalene emulsion adjuvant may consist essentially of squalene, polysorbate 80, sorbitan trioleate and water. Optionally the aqueous phase may contain additional components as desired or required depending upon the intended final presentation and vaccination strategy, such as buffers and/or tonicity modifying agents, in particular citrate ions e.g. 10 mM sodium citrate buffer.

Typically, the weight ratio of squalene to polysorbate 80 is 10 to 6.6, especially 9.1 to 7.5, in particular 8.7 to 7.9, such as 8.3.

Typically, the weight ratio of squalene to sorbitan trioleate is 10 to 6.6, especially 9.1 to 7.5, in particular 8.7 to 7.9, such as 8.3.

A single dose, such as a typical full human dose, of squalene emulsion adjuvant derived from MF59 may comprise 9 to 11 mg of squalene, such as 9.5 to 10 mg, in particular 9.75 mg. Higher or lower doses of squalene emulsion adjuvant derived from MF59 may be used. Suitably a single dose is at least 0.1× a typical full human dose, especially at least 0.25× a typical full human dose, in particular at least 0.5× a typical full human dose. Desirably the single dose is less than or equal to a full human dose. For example, the single dose may be 0.1 to 1× a typical full human dose, i.e. comprising 0.9 to 11 mg of squalene.

Particular single doses of interest include 0.1× a typical full human dose i.e. comprising 0.9 to 1.1 mg of squalene, 0.125× a typical full human dose i.e. comprising 1.1 to 1.4 mg of squalene, 0.25× a typical full human dose i.e. comprising 2.2 to 2.8 mg of squalene, such as 0.5× a typical full human dose i.e. comprising 4.5 to 5.5 mg of squalene or 1× a typical full human dose i.e. comprising 9 to 11 mg of squalene.

Squalene emulsion adjuvant derived from MF59 may include citrate ions e.g. 10 mM sodium citrate buffer.

The squalene emulsion adjuvant may be derived from AF03. Consequently, the squalene emulsion adjuvant may comprise squalene, sorbitan monooleate, polyoxyethylene cetostearyl ether and water. The squalene emulsion adjuvant may consist essentially of squalene, sorbitan monooleate, polyoxyethylene cetostearyl ether and water. Mannitol has been shown to reduce the phase transition temperature and is therefore desirable for manufacturing reasons, although excessive levels of mannitol may cause heterogeneity in size and larger droplets (Klucker, 2012). Optionally the aqueous phase may contain additional components as desired or required depending upon the intended final presentation and vaccination strategy, such as buffers and/or tonicity modifying agents, in particular phosphate buffered saline.

Typically, the weight ratio of squalene to sorbitan monooleate is 7.8 to 5.2, especially 7.15 to 5.85, in particular 6.8 to 6.2, such as 6.5.

Typically, the weight ratio of squalene to polyoxyethylene cetostearyl ether is 6.2 to 4.1, especially 5.7 to 4.7, in particular 5.4 to 4.9, such as 5.2.

Typically, the weight ratio of squalene to mannitol is 6.5 to 4.3, especially 5.9 to 4.9, in particular 5.7 to 5.1, such as 5.4.

A single dose, such as a typical full human dose, of squalene emulsion adjuvant derived from AF03 may comprise 11.2 to 13.6 mg of squalene, such as 12 to 12.8 mg, in particular 12.4 mg. Higher or lower doses of squalene emulsion adjuvant derived from AF03 may be used. Suitably a single dose is at least 0.1× a typical full human dose, especially at least 0.25× a typical full human dose, in particular at least 0.5× a typical full human dose. Desirably the single dose is less than or equal to a full human dose. For example, the single dose may be 0.1 to 1× a typical full human dose, i.e. comprising 1.1 to 13.6 mg of squalene.

Particular single doses of interest include 0.1× a typical full human dose i.e. comprising 1.1 to 1.35 mg of squalene, 0.125× a typical full human dose i.e. comprising 1.4 to 1.7 mg of squalene, 0.25× a typical full human dose i.e. comprising 2.8 to 3.4 mg of squalene, such as 0.5× a typical full human dose i.e. comprising 5.6 to 6.8 mg of squalene or 1× a typical full human dose i.e. comprising 11.2 to 13.6 mg of squalene.

Squalene emulsion adjuvant derived from AF03 may also include in particular phosphate buffered saline.

The squalene emulsion adjuvant may be derived from AS03. Consequently, the squalene emulsion adjuvant may comprise squalene, tocopherol, polysorbate 80 and water.

The squalene emulsion adjuvant may consist essentially of squalene, tocopherol, polysorbate 80 and water. Optionally the aqueous phase may contain additional components as desired or required depending upon the intended final presentation and vaccination strategy, such as buffers and/or tonicity modifying agents. Suitable buffers include Na₂HPO₄ and KH₂PO₄. Suitable tonicity modifying agents include NaCl and KCl. Modified phosphate buffered saline may be used, such as comprising Na₂HPO₄ and KH₂PO₄, NaCl and KCl.

Any of the α, β, γ, δ, ε or ξ tocopherols can be used, but α-tocopherol (also referred to herein as alpha-tocopherol) is typically used. D-alpha-tocopherol and D/L-alpha-tocopherol can both be used. A particularly desirable alpha-tocopherol is D/L-alpha-tocopherol.

Typically, the weight ratio of squalene to tocopherol is 0.5 to 1.5, especially 0.6 to 1.35, in particular 0.7 to 1.1, such as 0.85 to 0.95 e.g. 0.9. Suitably the tocopherol is alpha-tocopherol, such as D/L-alpha-tocopherol.

Typically, the weight ratio of squalene to polysorbate 80 is 1.2 to 3.6, especially 1.46 to 3.3, in particular 1.9 to 2.5 such as 2.1 to 2.3 e.g. 2.2.

A single dose, such as a typical full human dose, of squalene emulsion adjuvant derived from AS03 may comprise 9.7 to 12.1 mg of squalene, such as 10.1 to 11.8 mg, in particular 10.7 mg. Higher or lower doses of squalene emulsion adjuvant derived from AS03 may be used. Suitably a single dose is at least 0.1× a typical full human dose, especially at least 0.25× a typical full human dose, in particular at least 0.5× a typical full human dose. Desirably the single dose is less than or equal to a full human dose. For example, the single dose may be 0.1 to 1× a typical full human dose, i.e. comprising 0.9 to 12.1 mg of squalene.

Particular single doses of interest include 0.1× a typical full human dose i.e. comprising 0.9 to 1.3 mg of squalene (typically with 1 to 1.4 mg tocopherol, such as D/L-alpha tocopherol, and 0.43 to 0.57 mg polysorbate 80), 0.125× a typical full human dose i.e. comprising 1.2 to 1.6 mg of squalene (typically with 1.3 to 1.7 mg tocopherol, such as D/L-alpha tocopherol, and 0.54 to 0.71 mg polysorbate 80), 0.25× a typical full human dose i.e. comprising 2.4 to 3 mg of squalene (typically with 2.6 to 3.4 mg tocopherol, such as D/L-alpha tocopherol, and 1 to 1.5 mg polysorbate 80), such as 0.5× a typical full human dose i.e. comprising 4.8 to 6.1 mg of squalene (typically with 5.3 to 6.8 mg tocopherol, such as D/L-alpha tocopherol, and 2.1 to 2.9 mg polysorbate 80) or 1× a typical full human dose i.e. comprising 9.7 to 12.1 mg of squalene (typically with 10.6 to 13.6 mg tocopherol, such as D/L-alpha tocopherol, and 4.3 to 5.7 mg polysorbate 80).

Squalene emulsion adjuvant derived from AS03 may also include in particular a phosphate buffered saline, such as modified phosphate buffered saline.

The squalene emulsion adjuvant may be derived from SE. Consequently, the squalene emulsion adjuvant may comprise squalene, phosphatidyl choline, poloxamer 188 and water, optionally with glycerol. The squalene emulsion adjuvant may consist essentially of squalene, phosphatidyl choline, poloxamer 188 and water, optionally with glycerol. Optionally the aqueous phase may contain additional components as desired or required depending upon the intended final presentation and vaccination strategy, such as buffers and/or tonicity modifying agents, in particular ammonium phosphate buffer. Tocopherol, such as alpha-tocopherol may be present as an antioxidant.

Typically, the weight ratio of squalene to phosphatidyl choline is 2.52 to 3.8, especially 2.85 to 3.5, in particular 3 to 3.3, such as 3.15.

Typically, the weight ratio of squalene to poloxamer 188 is 55 to 83, especially 62 to 76, in particular 65.5 to 72.5, such as 69.

Typically, the weight ratio of squalene to tocopherol, if present, is at least 50, especially 137 to 207, in particular 154 to 190, such as 163 to 181, for example 172.

A single dose, such as a typical full human dose, of squalene emulsion adjuvant derived from SE may comprise 7.7 to 9.5 mg of squalene, such as 8.1 to 9 mg, in particular 8.6 mg. Higher or lower doses of squalene emulsion adjuvant derived from SE may be used.

Suitably a single dose is at least 0.1× a typical full human dose, especially at least 0.25× a typical full human dose, in particular at least 0.5× a typical full human dose. Desirably the single dose is less than or equal to a full human dose. For example, the single dose may be 0.1 to 1× a typical full human dose, i.e. comprising 0.77 to 9.5 mg of squalene.

Particular single doses of interest include 0.1× a typical full human dose i.e. comprising 0.77 to 0.95 mg of squalene, 0.125× a typical full human dose i.e. comprising 0.96 to 1.2 mg of squalene, 0.25× a typical full human dose i.e. comprising 1.9 to 2.4 mg of squalene, such as 0.5× a typical full human dose i.e. comprising 3.8 to 4.8 mg of squalene or 1× a typical full human dose i.e. comprising 7.7 to 9.5 mg of squalene.

Squalene emulsion adjuvant derived from SE may also include in particular ammonium phosphate buffer and glycerol.

The squalene emulsion adjuvant may be derived from SEA160. Consequently, the squalene emulsion adjuvant may comprise squalene, polysorbate 80, sorbitan trioleate and water. The squalene emulsion adjuvant may consist essentially of squalene, polysorbate 80, sorbitan trioleate and water. Optionally the aqueous phase may contain additional components as desired or required depending upon the intended final presentation and vaccination strategy, such as buffers and/or tonicity modifying agents.

Typically, the weight ratio of squalene to polysorbate 80 is 4.6 to 3.0, especially 4.2 to 3.4, in particular 4.0 to 3.6, such as 3.8.

Typically, the weight ratio of squalene to sorbitan trioleate is 4.6 to 3.0, especially 4.2 to 3.4, in particular 4.0 to 3.6, such as 3.8.

A single dose, such as a typical full human dose, of squalene emulsion adjuvant derived from SEA160 may comprise 6.8 to 8.4 mg of squalene, such as 7.2 to 8 mg, in particular 7.6 mg. Higher or lower doses of squalene emulsion adjuvant derived from SEA160 may be used. Suitably a single dose is at least 0.1× a typical full human dose, especially at least 0.25× a typical full human dose, in particular at least 0.5× a typical full human dose. Desirably the single dose is less than or equal to a full human dose. For example, the single dose may be 0.1 to 1× a typical full human dose, i.e. comprising 0.68 to 8.4 mg of squalene.

Particular single doses of interest include 0.1× a typical full human dose i.e. comprising 0.68 to 0.84 mg of squalene, 0.125× a typical full human dose i.e. comprising 0.85 to 1.1 mg of squalene, 0.25× a typical full human dose i.e. comprising 1.7 to 2.1 mg of squalene, such as 0.5× a typical full human dose i.e. comprising 3.4 to 4.2 mg of squalene or 1× a typical full human dose i.e. comprising 6.8 to 8.4 mg of squalene.

Squalene emulsion adjuvant derived from SEA160 may also include in particular a phosphate buffered saline, such as modified phosphate buffered saline.

The squalene emulsion adjuvant may be derived from SEAS44. Consequently, the squalene emulsion adjuvant may comprise squalene, tocopherol, polysorbate 80 and water. The squalene emulsion adjuvant may consist essentially of squalene, tocopherol, polysorbate 80 and water. Optionally the aqueous phase may contain additional components as desired or required depending upon the intended final presentation and vaccination strategy, such as buffers and/or tonicity modifying agents. Suitable buffers include Na₂HPO₄ and KH₂PO₄. Suitable tonicity modifying agents include NaCl and KCl. Modified phosphate buffered saline may be used, such as comprising Na₂HPO₄ and KH₂PO₄, NaCl and KCl.

Any of the α, β, γ, δ, ε or ξ tocopherols can be used, but α-tocopherol is typically used. D-alpha-tocopherol and D/L-alpha-tocopherol can both be used. A particularly desirable alpha-tocopherol is D/L-alpha-tocopherol.

Typically, the weight ratio of squalene to tocopherol is 2.6 to 4.5, especially 2.8 to 4.3, in particular 3.25 to 4, such as 3.4 to 3.8 e.g. 3.6. Suitably the tocopherol is alpha-tocopherol, especially D/L-alpha-tocopherol.

Typically, the weight ratio of squalene to polysorbate 80 is 1.3 to 2.5, especially 1.56 to 2.3, in particular 1.75 to 2.15 such as 1.85 to 2 e.g. 1.94.

A single dose, such as a typical full human dose, of squalene emulsion adjuvant derived from SEAS44 may comprise 11.7 to 14.3 mg of squalene, such as 12.3 to 13.7 mg, in particular 13 mg. Higher or lower doses of squalene emulsion adjuvant derived from SEAS44 may be used. Suitably a single dose is at least 0.1× a typical full human dose, especially at least 0.25× a typical full human dose, in particular at least 0.5× a typical full human dose. Desirably the single dose is less than or equal to a full human dose. For example, the single dose may be 0.1 to 1× a typical full human dose, i.e. comprising 1.1 to 14.3 mg of squalene.

Particular single doses of interest include 0.1× a typical full human dose i.e. comprising 1.1 to 1.5 mg of squalene, 0.125× a typical full human dose i.e. comprising 1.4 to 1.8 mg of squalene, 0.25× a typical full human dose i.e. comprising 2.9 to 3.6 mg of squalene, such as 0.5× a typical full human dose i.e. comprising 5.8 to 7.2 mg of squalene or 1× a typical full human dose i.e. comprising 11.7 to 14.3 mg of squalene.

Squalene emulsion adjuvant derived from SEAS44 may also include in particular a phosphate buffered saline, such as modified phosphate buffered saline.

Self-emulsifying adjuvants, such as SEA160, SEAS44 and squalene emulsion adjuvant adjuvants derived therefrom, may be provided in dry form. For example, such dry self-emulsifying adjuvants may consist essentially of squalene and surfactant(s), such as in the case of SEA160 derived squalene emulsion adjuvants. Such dry self-emulsifying adjuvants may consist essentially of squalene and surfactant(s) or consist essentially of squalene, tocopherol and surfactant(s), such as in the case of SEAS44 derived tocopherol containing squalene emulsion adjuvants.

High pressure homogenization (HPH or microfluidisation) may be applied to yield squalene emulsion adjuvants which demonstrate uniformly small droplet sizes and long-term stability (see EP 0 868 918 B1 and WO2006/100109). Briefly, oil phase composed of squalene and tocopherol may be formulated under a nitrogen atmosphere. Aqueous phase is prepared separately, typically composed of water for injection or phosphate buffered saline, and polysorbate 80. Oil and aqueous phases are combined, such as at a ratio of 1:9 (volume of oil phase to volume of aqueous phase) before homogenisation and microfluidisation, such as by a single pass through an in-line homogeniser and three passes through a microfluidiser (at around 15000 psi). The resulting emulsion may then be sterile filtered, for example through two trains of two 0.5/0.2 um filters in series (i.e. 0.5/0.2/0.5/0.2), see WO2011/154444. Operation is desirably undertaken under an inert atmosphere, e.g. nitrogen. Positive pressure may be applied, see WO2011/154443.

WO2015/140138, WO2016/135154, Shah, 2014 Shah, 2015, Shah, 2019, international patent application PCT/US2020/15565 (published as WO2020/160080) and Lodaya, 2019 describe squalene emulsion adjuvants which are self-emulsifying adjuvant systems (SEAS) and their manufacture.

### Subjects

The present invention is generally intended for mammalian subjects, in particular human subjects. The subject may be a wild or domesticated animal. Mammalian subjects include for example cats, dogs, pigs, sheep, horses or cattle. In one embodiment the invention, the subject is human.

The subject to be treated using the method of the invention may be of any age.

In one embodiment the subject is a human infant (up to 12 months of age). In one embodiment the subject is a human child (less than 18 years of age). In another embodiment the subject is a human child between the ages of 6 months up to 11 years, 5 years up to 11 years, 12 years up to 16 or 17 years. In one embodiment the subject is an adult human (aged 18-59). In one embodiment the subject is an older human (aged 60 or greater).

Doses administered to younger children, such as less than 12 years of age, may be reduced relative to an equivalent adult dose, such as by 50%.

The methods of the invention are suitably intended for prophylaxis of infectious diseases, i.e. for administration to a subject which is not infected with a pathogen. For example methods of the invention are suitably intended for prophylaxis of SARS-CoV-2 infection, i.e. for administration to a subject which is not infected with SARS-CoV-2.

In other embodiments the methods of the invention may be intended for treatment, e.g. for the treatment of infectious diseases, i.e. for administration to a subject which is infected with a pathogen.

### Antigen

According to the present invention, the squalene emulsion adjuvants are to be utilised in conjunction with carrier-formulated mRNA wherein the mRNA encodes an antigen (also referred to herein simply as ‘carrier-formulated mRNA’). By the term ‘carrier-formulated mRNA’ is meant mRNA that is formulated with a carrier to facilitate delivery and/or improved stability. Suitable carriers include LNP, CNE and LION.

By the term antigen is meant a polypeptide which is capable of eliciting an immune response in a subject. Suitably the immune response is a protective immune response, e.g. reducing partially or completely the severity of one or more symptoms and/or time over which one or more symptoms are experienced by a subject, reducing the likelihood of developing an established infection after challenge and/or slowing progression of an associated illness (e.g. extending survival).

Suitably the antigen comprises at least one B or T cell epitope, suitably an antigen comprises B and T cell epitopes. The elicited immune response may be an antigen specific B cell response which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

Suitably the encoded antigen contains 3000 residues or fewer, especially 2000 residues or fewer, in particular 1500 residues or fewer. The encoded antigen may contain 1000 residues or fewer, 800 residues or fewer, 600 residues or fewer, 400 residues or fewer or 200 residues or fewer.

Suitably the antigen contains 50 residues or more, especially 100 residues or more, in particular 150 residues or more.

Suitably the antigen contains 50 to 3000 residues, especially 100 to 1500 residues, in particular 200 to 1000 residues.

The antigen may be derived from a pathogen, especially a human pathogen, (such as a bacterium, virus or parasite) or may be a cancer antigen (such as a tumour antigen and/or a neoantigen).

In one embodiment, the antigen is derived from a coronavirus, particularly from SARS-CoV-2. A plurality of antigens maybe encoded. Consequently, in some embodiments the antigen is derived from at least one coronavirus, for example from SARS-CoV-2. In some embodiments the antigen is derived from more than one coronavirus (such as 2, 3, 4 or 5), for example from SARS-CoV-2 (such as a plurality of SARS-CoV-2 variant antigens).

SARS-CoV-2 makes use of a densely glycosylated spike (S) protein to gain entry into host cells. In coronaviruses, the S protein is a trimeric class I fusion protein which exists in a metastable pre-fusion conformation that undergoes a substantial structural rearrangement to fuse the viral membrane with the host cell membrane (Li, 2016; Bosch, 2003).

A coronavirus protein of use in the present invention may be a fragment or variant of a native coronavirus protein which is capable of eliciting neutralising antibodies and/or a T cell response (such as a CD4 or CD8 T cell response) to a coronavirus, suitably a protective immune response.

A SARS-CoV-2 S protein of use in the present invention comprises, such as consists of, a fragment or variant of a native SARS-CoV-2 S protein which is capable of eliciting neutralising antibodies and/or a T cell response (such as a CD4 or CD8 T cell response) to SARS-CoV-2, suitably a protective immune response.

The encoded SARS-CoV-2 S protein may comprise, such as consist of, a full length S protein (such as SEQ ID NO:1). Alternatively, the encoded SARS-CoV-2 S protein may comprise, such as consist of, an amino acid sequence having at least 90% identity to the amino acid sequence set forth in SEQ ID NO:1. The encoded SARS-CoV-2 S protein may comprise, such as consist of, an amino acid sequence having at least 95% identity to the amino acid sequence set forth in SEQ ID NO:1, especially at least 98% identity to the amino acid sequence set forth in SEQ ID NO:1, in particular at least 99% identity to the amino acid sequence set forth in SEQ ID NO:1, such as 100% identity to the amino acid sequence set forth in SEQ ID NO:1.

The encoded SARS-CoV-2 S protein may comprise, or consist of, one or more domains of a full length SARS-CoV-2 S protein, such as the ectodomain (SEQ ID NO:2) or receptor binding domain (RBD, SEQ ID NO:3), or variants thereof.

The encoded SARS-CoV-2 S protein may comprise, or consist of, an amino acid sequence having at least 90% identity to the amino acid sequence set forth in SEQ ID NO:2. The encoded SARS-CoV-2 S protein may comprise, or consist of, an amino acid sequence having at least 95% identity to the amino acid sequence set forth in SEQ ID NO:2, especially at least 98% identity to the amino acid sequence set forth in SEQ ID NO:2, in particular at least 99% identity to the amino acid sequence set forth in SEQ ID NO:2, such as 100% identity to the amino acid sequence set forth in SEQ ID NO:2.

The encoded SARS-CoV-2 S protein may comprise, such as consist of, an amino acid sequence having at least 90% identity to the amino acid sequence set forth in SEQ ID NO:3. The encoded SARS-CoV-2 S protein may comprise, such as consist of, an amino acid sequence having at least 95% identity to the amino acid sequence set forth in SEQ ID NO:3, especially at least 98% identity to the amino acid sequence set forth in SEQ ID NO:3, in particular at least 99% identity to the amino acid sequence set forth in SEQ ID NO:3, such as 100% identity to the amino acid sequence set forth in SEQ ID NO:3.

Suitably the encoded SARS-CoV-2 S protein is pre-fusion stabilised to facilitate appropriate presentation to the immune system. For example, Wrapp and colleagues (Wrapp et al., 2020) produced a recombinant prefusion S ectodomain using a stabilization strategy that proved effective for other betacoronavirus S proteins (Pallesen et al, 2017; Kirchdoerfer et al, 2018). To this end, starting with the SARS-CoV-2 polynucleotide sequence (GenBank accession number MN908947.3), a gene encoding residues 1 to 1208 of SARS-CoV-2 S protein (UniProt accession number PODTC2 version 1 dated 22 Apr. 2020) with proline substitutions at residues 986 and 987, a “GSAS” substitution at the furin cleavage site (residues 682-685) a C-terminal T4 fibritin trimerization motif, an HRV3C protease cleavage site, a TwinStrepTag and an 8×HisTag was synthesized and cloned into the mammalian expression vector pαH.

Residues 1 to 1208 of SARS-CoV-2 S protein with proline substitutions at residues 986 and 987, a “GSAS” substitution at the furin cleavage site are provided in SEQ ID NO:4, which is an example of a pre-fusion stabilized ectodomain of SARS-CoV-2 S protein.

The encoded SARS-CoV-2 S protein may comprise, or consist of, an amino acid sequence having at least 90% identity to the amino acid sequence set forth in SEQ ID NO:4. The encoded SARS-CoV-2 S protein may comprise, such as consist of, an amino acid sequence having at least 95% identity to the amino acid sequence set forth in SEQ ID NO:4, especially at least 98% identity to the amino acid sequence set forth in SEQ ID NO:4, in particular at least 99% identity to the amino acid sequence set forth in SEQ ID NO:4, such as 100% identity to the amino acid sequence set forth in SEQ ID NO:4.

Suitably the SARS-CoV-2 S protein is a pre-fusion stabilised protein.

In one embodiment, the SARS-CoV-2 S protein is the stabilized recombinant prefusion S ectodomain disclosed by Wrapp et al., 2020.

The SARS-CoV-2 S protein (such as a pre-fusion stabilized SARS-CoV-2 S protein) may desirably be in the form of a trimer and consequently may comprise a trimerization motif, such as a T4 fibritin trimerization motif, more suitably a C-terminal T4 fibritin trimerization motif. Alternative trimerization motifs include, for example, a domain derived from collagen called ‘Trimer-Tag’ such as disclosed in Liu et al., 2017, or a molecular clamp, such as that disclosed in WO2018/176103.

An encoded SARS-CoV-2 S protein is desirably 1800 residues or fewer in length, especially 1500 residues or fewer, in particular 1400 residues or fewer, such as 1300 residues or fewer.

An encoded SARS-CoV-2 S protein is desirably 150 residues or more in length, especially 200 residues or more, in particular 400 residues or more, such as 600 residues or more.

In one embodiment the antigen is a human cytomegalovirus (CMV) antigen.

In one embodiment the antigen is a Zika virus antigen.

In one embodiment the antigen is a human parainfluenza virus (PIV) antigen, such as a human PIV type 3 antigen.

In one embodiment the antigen is a human metapneumovirus (hMPV) antigen.

In one embodiment the antigen is a respiratory syncytial virus (RSV) antigen.

In one embodiment the antigen is an influenza virus antigen, such as a hemagglutinin or a neuraminidase.

In one embodiment the antigen an Epstein-Barr virus (EBV) antigen.

In one embodiment the antigen is a Herpes simplex virus (HSV) antigen, such as a gE and/or a gl antigen. The gE antigen may be a sequence comprising, or consisting of, a sequence having at least 80%, such as at least 90%, especially at least 95%, in particular at least 98% for example at least 99% or 100% identity to SEQ ID No: 9. The gl antigen may be a sequence comprising, or consisting of, a sequence having at least 80%, such as at least 90%, especially at least 95%, in particular at least 98% for example at least 99% or 100% identity to SEQ ID No: 10.

A Herpes simplex virus (HSV) gE antigen may be a sequence comprising, such as consisting of, a sequence having at least 80%, such as at least 90%, especially at least 95%, in particular at least 98% for example at least 99% or 100% identity to SEQ ID No: 13.

mRNA

Messenger RNA (mRNA) can direct the cellular machinery of a subject to produce proteins. mRNA may be circular or branched, but will generally be linear.

mRNA used herein are preferably provided in purified or substantially purified form i.e. substantially free from proteins (e.g., enzymes), other nucleic acids (e.g. DNA and nucleoside phosphate monomers), and the like, generally being at least about 50% pure (by weight), and usually at least 90% pure, such as at least 95% or at least 98% pure.

mRNA may be prepared in many ways e.g. by chemical synthesis in whole or in part, by digesting longer nucleic acids using nucleases (e.g. restriction enzymes), by joining shorter nucleic acids or nucleotides (e.g. using ligases or polymerases), from genomic or cDNA libraries, etc. In particular, mRNA may be prepared enzymatically using a DNA template.

The term mRNA as used herein includes conventional mRNA or mRNA analogs, such as those containing modified backbones or modified bases (e.g. pseudouridine, or the like). mRNA, may or may not have a 5′ cap.

The mRNA comprises a sequence which encodes at least one antigen. Typically, the nucleic acids of the invention will be in recombinant form, i.e. a form which does not occur in nature. For example, the mRNA may comprise one or more heterologous nucleic acid sequences (e.g. a sequence encoding another antigen and/or a control sequence such as a promoter or an internal ribosome entry site) in addition to the sequence encoding the antigen.

Alternatively, or in addition, the sequence or chemical structure of the nucleic acid may be modified compared to a naturally-occurring sequence which encodes the antigen. The sequence of the nucleic acid molecule may be modified, e.g. to increase the efficacy of expression or replication of the nucleic acid, or to provide additional stability or resistance to degradation.

mRNA may also be codon optimised. In some embodiments, mRNA may be codon optimised for expression in human cells. By “codon optimised” is intended modification with respect to codon usage which may increase translation efficacy and/or half-life of the nucleic acid.

A poly A tail (e.g., of about 30 adenosine residues or more) may be attached to the 3′ end of the RNA to increase its half-life.

The 5 end of the RNA may be capped, for example with a modified ribonucleotide with the structure m7G (5) ppp (5) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methyltransferase, which catalyzes the construction of N7-monomethylated cap 0 structures). Cap 0 structure plays an important role in maintaining the stability and translational efficacy of the mRNA molecule. The 5′ cap of the mRNA molecule may be further modified by a 2′-O-Methyltransferase which results in the generation of a cap 1 structure (m7Gppp [m2′-O] N), which may further increase translation efficacy.

mRNA may comprise one or more nucleotide analogs or modified nucleotides. As used herein, “nucleotide analog” or “modified nucleotide” refers to a nucleotide that contains one or more chemical modifications (e.g., substitutions) in or on the nitrogenous base of the nucleoside (e.g. cytosine (C), thymine (T) or uracil (U)), adenine (A) or guanine (G)). A nucleotide analog can contain further chemical modifications in or on the sugar moiety of the nucleoside (e.g. ribose, modified ribose, six-membered sugar analog, or open-chain sugar analog), or the phosphate. The preparation of nucleotides and modified nucleotides and nucleosides are well-known in the art, see the following references: U.S. Pat. Nos. 4,373,071, 4,458,066, 4,500,707, 4,668,777, 4,973,679, 5,047,524, 5,132,418, 5,153,319, 5,262,530, 5,700,642. Many modified nucleosides and modified nucleotides are commercially available.

Modified nucleobases (chemical modifications) which can be incorporated into modified nucleosides and nucleotides and be present in the mRNA molecules include: m5C (5-methylcytidine); m5U (5-methyluridine); m6A (N6-methyladenosine); s2U (2-thiouridine); Um (2′-O-methyluridine); m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); m′l (1-methylinosine); m′lm (I,2′-O-dimethylinosine); m3C (3-methylcytidine); Cm (2′-O-methylcytidine); s2C (2-thiocytidine); ac4C (N4-acetylcytidine); f5C (5-fonnylcytidine); m5Cm (5,2-O-dimethylcytidine); ac4Cm (N4-acetyl-2-O-methylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-O-methyl uridine); cmnm5s2U (5-carboxymethylaminomethyl-2-thiouridine); m62A (N6,N6-dimethyladenosine); Im (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,2′-O-dimethyladenosine); rn62Am (N6,N6,0-2-trimethyladenosine); m2′7G (N2,7-dimethylguanosine); m2′2′7G (N2,N2,7-trimethylguanosine); m3Um (3,2′-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C₂-C₆)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Many of these modified nucleobases and their corresponding ribonucleosides are available from commercial suppliers.

The mRNA may encode more than one antigen. For example, the mRNA encoding an antigen protein may encode only the antigen or may encode additional proteins. Each antigen and additional protein(s) may be under the control of different regulatory elements. Alternatively, the antigen and additional proteins may be under the control of the same regulatory element. Where at least two additional proteins are encoded, some of the antigen and additional proteins may be under the control of the same regulatory element and some may be under the control of different regulatory elements.

mRNA may be non-replicating or may be replicating, also known as self-replicating. A self-replicating mRNA molecule may be an alphavirus-derived mRNA replicon. mRNA amplification can also be achieved by the provision of a non-replicating mRNA encoding an antigen in conjunction with a separate mRNA encoding replication machinery.

Self-replicating RNA molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides an RNA-dependent RNA polymerase which then produces both antisense and sense transcripts from the delivered RNA. Thus the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells.

Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons, see the following reference: WO2005/113782.

In certain embodiments, the self-replicating RNA molecule described herein encodes (i) an RNA-dependent RNA polymerase which can transcribe RNA from the self-replicating RNA molecule and (ii) an antigen. The polymerase can be an alphavirus replicase e.g. comprising one or more of alphavirus proteins nsPI, nsP2, nsP3 and nsP4.

Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins.

Thus, a self-replicating RNA molecule useful with the invention may have two open reading frames. The first (5) open reading frame encodes a replicase; the second (3) open reading frame encodes an antigen. In some embodiments the RNA may have additional (e.g. downstream) open reading frames e.g. to encode further antigens or to encode accessory polypeptides.

In certain embodiments, the self-replicating RNA molecule disclosed herein has a 5′ cap (e.g. a 7-methylguanosine). This cap can enhance in vivo translation of the RNA. In some embodiments the 5′ sequence of the self-replicating RNA molecule must be selected to ensure compatibility with the encoded replicase.

A self-replicating RNA molecule may have a 3′ poly-A tail. It may also include a poly-A polymerase recognition sequence (e.g. AAUAAA) near its 3′ end.

Self-replicating RNA molecules can have various lengths, but they are typically 5000 to 25000 nucleotides long, such as 8000 to 15000 nucleotides long, for example 9000 to 12000 nucleotides long. Self-replicating RNA molecules will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA. In another embodiment, a self-replicating RNA may comprise two separate RNA molecules, each comprising a nucleotide sequence derived from an alphavirus: one RNA molecule comprises an RNA construct for expressing alphavirus replicase, and one RNA molecule comprises an RNA replicon that can be replicated by the replicase in trans. In some embodiments, the RNA construct for expressing alphavirus replicase comprises a 5′-cap. See WO2017/162265.

The self-replicating RNA can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-replicating RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-replicating RNA can include (in addition to any 5′ cap structure) one or more nucleotides having a modified nucleobase. An RNA used with the invention ideally includes only phosphodiester linkages between nucleosides, but in some embodiments it can contain phosphoramidate, and/or methylphosphonate linkages.

The self-replicating RNA molecule may encode a single heterologous polypeptide antigen (i.e. the antigen) or, optionally, two or more heterologous polypeptide antigens linked together in a way that each of the sequences retains its identity (e.g., linked in series) when expressed as an amino acid sequence. The heterologous polypeptides generated from the self-replicating RNA may then be produced as a fusion polypeptide or engineered in such a manner to result in separate polypeptide or peptide sequences.

The self-replicating RNA molecules described herein may be engineered to express multiple nucleotide sequences, from two or more open reading frames, thereby allowing co-expression of proteins, such as one, two or more Coronavirus antigens (e.g. one, two or more coronavirus protein(s), such as one, two or more SARS-CoV-2 S protein(s)) together with cytokines or other immunomodulators, which can enhance the generation of an immune response. Such a self-replicating RNA molecule might be particularly useful, for example, in the production of various gene products (e.g., proteins) at the same time, for example, as a bivalent or multivalent vaccine.

If desired, the self-replicating RNA molecules can be screened or analyzed to confirm their therapeutic and prophylactic properties using various in vitro or in vivo testing methods that are known to those of skill in the art. For example, vaccines comprising self-replicating RNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self-replicating RNA molecule that encodes an antigen. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry.

Self-replicating RNA molecules that encode an antigen can also be tested for ability to induce humoral immune responses, as evidenced, for example, by induction of B cell production of antibodies specific for the antigen of interest. These assays can be conducted using, for example, peripheral B lymphocytes from immunized individuals. Such assay methods are known to those of skill in the art. Other assays that can be used to characterize the self-replicating RNA molecules can involve detecting expression of the encoded antigen by the target cells. For example, FACS can be used to detect antigen expression on the cell surface or intracellularly. Another advantage of FACS selection is that one can sort for different levels of expression; sometimes-lower expression may be desired. Other suitable method for identifying cells which express a particular antigen involve panning using monoclonal antibodies on a plate or capture using magnetic beads coated with monoclonal antibodies.

A non-replicating mRNA will typically contain 10000 bases or fewer, especially 8000 bases or fewer, in particular 5000 bases or fewer. A replicating mRNA will typically contain 25000 bases or fewer, especially 20000 bases or fewer, in particular 15000 bases or fewer. A replicating mRNA may contain 5000 to 25000 nucleotides, such as 8000 to 15000 nucleotides, for example 9000 to 12000 nucleotides.

A single dose of mRNA may be 0.001 to 1000 ug, especially 1 to 500 ug, in particular 10 to 250 ug. A single dose of mRNA may be 0.001 to 75 ug, 1 to 75 ug, 25 to 250 ug, or 250 to 1000 ug. Specifically, a replicating mRNA dose may be 0.001 to 75 ug, such as 0.1 to 75 ug. A non-replicating mRNA dose may, for example, be 1 to 500 ug, such as 1 to 250 ug.

In one embodiment the mRNA is non-replicating mRNA. In a second embodiment the mRNA is replicating mRNA.

mRNA Carriers

A range of carrier systems have been described which encapsulate or complex mRNA in order to facilitate mRNA delivery and consequent expression of encoded antigens as compared to mRNA which is not encapsulated or complexed. The present invention may utilise any suitable carrier system. Particular mRNA carrier systems of note are further described below.

### LNP

Lipid nanoparticles (LNPs) are non-virion liposome particles in which mRNA can be encapsulated. LNP delivery systems and methods for their preparation are known in the art. The particles can include some external mRNA (e.g. on the surface of the particles), but desirably at least half of the RNA (and suitably at least 85%, especially at least 95%, such as all of it) is encapsulated.

LNP formulated mRNA may be prepared comprising mRNA, cationic lipid, and other helper lipids. Liposomal particles can, for example, be formed of a mixture of zwitterionic, cationic and anionic lipids which can be saturated or unsaturated, for example; DSPC (zwitterionic, saturated), DlinDMA (cationic, unsaturated), and/or DMG (anionic, saturated). Preferred LNPs for use with the invention include an amphiphilic lipid which can form liposomes, optionally in combination with at least one cationic lipid (such as DOTAP, DSDMA, DODMA, DLinDMA, DLenDMA, etc.). A mixture of DSPC, DlinDMA, PEG-DMG and cholesterol is particularly effective. Other useful LNPs are described in the following references: WO 2012/006372, WO2012/006376; WO2012/030901; WO2012/031046: WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. September 4; 109(36): 14604-9.

LNP can, for example, be formed of a mixture of (i) a PEG-modified lipid (ii) a non-cationic lipid (iii) a sterol (iv) an ionisable cationic lipid. Alternatively, LNP can for example be formed of a mixture of (i) a PEG-modified lipid (ii) a non-cationic lipid (iii) a sterol (iv) a non-ionisable cationic lipid.

The PEG-modified lipid may comprise a PEG molecule with a molecular weight of 10000 Da or less, especially 5000 Da or less, in particular 3000 Da, such as 2000 Da or less. Examples of PEG-modified lipids include PEG-distearoyl glycerol, PEG-dipalmitoyl glycerol and PEG-dimyristoyl glycerol. The PEG-modified lipid is typically present at around 0.5 to 15 molar %.

The non-cationic lipid may be a neutral lipid, such as 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE) and sphingomyelin (SM). The non-cationic lipid is typically present at around 5 to 25 molar %.

The sterol may be cholesterol. The sterol is typically present at around 25 to 55 molar %.

A range of suitable ionizable cationic lipids are known in the art, which are typically present at around 20 to 60 molar %.

The ratio of RNA to lipid can be varied (see for example WO2013/006825). “N:P ratio” refers to the molar ratio of protonatable nitrogen atoms in the cationic lipids (typically solely in the lipid's headgroup) to phosphates in the RNA. The ratio of nucleotide (N) to phospholipid (P) can be in the range of, e.g., 1N:1P to 20N:1P, 1N:1P to 10N:1P, 2N:1P to 8N:1P, 2N:1P to 6N:1P or 3N:1P to 5N:1P. The ratio of nucleotide (N) to phospholipid (P) can be in the range of, e.g., 1N:1P, 2N:1P, 3N:1P, 4N:1P, 5N:1P, 6N:1P, 7N:1P, 8N:1P, 9N:1P, or 10N:1P. Alternatively or additionally, the ratio of nucleotide (N) to phospholipid (P) is 4N:1P.

WO2017/070620 provides general information on LNP compositions and is incorporated herein by reference. Other useful LNPs are described in the following references: WO2012/006376 (LNP and microparticle delivery systems); WO2012/030901; WO2012/031046; WO2012/031043; WO2012/006378; WO2011/076807; WO2013/033563; WO2013/006825; WO2014/136086; WO2015/095340; WO2015/095346; WO2016/037053, which are also incorporated herein by reference. Other useful LNPs are described in WO2012/006359 (microparticle delivery systems) which is also incorporated herein by reference.

LNP delivery systems, and methods for their preparation are described in the following reference: Geall et al. (2012) PNAS USA. September 4; 109(36): 14604-9 (LNP delivery system), which is also incorporated herein by reference.

LNPs are typically 50 to 200 um in diameter (Z-average). Suitably the LNPs have a polydispersity of 0.4 or less, such as 0.3 or less.

In one embodiment the carrier is a lipid nanoparticle (LNP).

### CNE

The carrier may be a cationic nanoemulsion (CNE) delivery system. Such cationic oil-in-water emulsions can be used to deliver the mRNA to the interior of a cell. The emulsion particles comprise a hydrophobic oil core and a cationic lipid, the latter of which can interact with the mRNA, thereby anchoring it to the emulsion particle. In a CNE delivery system, the mRNA which encodes the antigen is complexed with a particle of a cationic oil-in-water emulsion. CNE carriers and methods for their preparation are described in WO2012/006380, WO2013/006837 and WO2013/006834 which are incorporated herein by reference.

Thus, the mRNA may be complexed with a particle of a cationic oil-in-water emulsion. The particles typically comprise an oil core (e.g. a plant oil or squalene) that is in liquid phase at 25° C., a cationic lipid (e.g. phospholipid) and, optionally, a surfactant (e.g. sorbitan trioleate, polysorbate 80); polyethylene glycol can also be included. Alternatively or additionally, the CNE comprises squalene and a cationic lipid, such as 1,2-dioleoyloxy-3-(trimethylammonium)propane (DOTAP) (see e.g. Brito, 2014). In an embodiment, the CNE is an oil-in-water emulsion of DOTAP and squalene stabilised with polysorbate 80 and/or sorbitan trioleate.

Desirably at least half of the RNA (and suitably at least 85%, such as all of it) is complexed with the cationic oil-in-water emulsion carrier.

CNE are typically 50 to 200 um in diameter (Z-average). Suitably the CNE have a polydispersity of 0.4 or less, such as 0.3 or less.

In one embodiment the carrier is a CNE.

### LION

A lipidoid-coated iron oxide nanoparticle (LION) is capable of delivering mRNA into cells and may be aided after administration to a subject by application of an external magnetic field. A LION is an iron oxide particle with one or more coatings comprising lipids and/or lipidoids wherein mRNA encoding the antigen is incorporated into or associated with the lipid and/or lipidoid coating(s) through electrostatic interactions. The mRNA being embedded within the coating(s) may offer protection from enzymatic degradation. The lipids and/or lipidoids comprised within a LION may for example include those included in Figure S1 of Jiang, 2013, especially lipidoids comprising alkyl tails of 12 to 14 carbons in length and in particular lipidoid C14-200 as disclosed in Jiang, 2013. A LION may typically comprise 200 to 5000, such as 500 to 2000, in particular about 1000 lipid and/or lipidoid molecules. Typically the LIONs are 20 to 200 nm in diameter, especially 50 to 100 nm in diameter. The lipid/lipidoid to mRNA weight ratio may be about 1:1 to 10:1, especially about 5:1. Particularly suitable LIONs, and methods for preparation of LIONs are disclosed in Jiang, 2013.

In one embodiment the carrier is a lipidoid-coated iron oxide nanoparticle (LION).

### Sequence Alignments

Identity or homology with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences.

### Additional Antigens

The present invention may involve a plurality of antigenic components, for example with the objective to elicit a broad immune response e.g. to a pathogen, such as a coronavirus, or to elicit responses to multiple pathogens. Consequently, more than one antigen may be present, more than one polynucleotide encoding an antigen may be present, one polynucleotide encoding more than one antigen may be present or a mixture of antigen(s) and polynucleotide(s) encoding antigen(s) may be present. Polysaccharides such as polysaccharide conjugates, may also be present.

### Formulation and Administration

The carrier-formulated mRNA and squalene emulsion adjuvant may be administered as a formulation containing the carrier-formulated mRNA and squalene emulsion adjuvant (‘co-formulation’ or ‘co-formulated’). Alternatively, the carrier-formulated mRNA and squalene emulsion adjuvant may be administered as two or more formulations, for example, a first formulation containing the carrier-formulated mRNA and a second formulation containing the squalene emulsion adjuvant (‘separate formulation’ or ‘separately formulated’). Consequently, it will be appreciated that a range of formulation possibilities exist. A reasonable balance is desirable between practical considerations such as: compatibility of components for co-formulation; manufacture, storage and distribution of a plurality of single vs multiple component formulations; and the need for multiple administrations in the case of separate formulation.

When separately formulated, the carrier-formulated mRNA and squalene emulsion adjuvant may be administered through the same or different routes, to the same or different locations, and at the same or different times.

The carrier-formulated mRNA and squalene emulsion adjuvant may be administered via various suitable routes, including parenteral, such as intramuscular or subcutaneous administration. The carrier-formulated mRNA and squalene emulsion adjuvant may be administered via different routes. Suitably the carrier-formulated mRNA and squalene emulsion adjuvant are administered via the same route, in particular intramuscularly.

When administered as separate formulations, the carrier-formulated mRNA and squalene emulsion adjuvant are desirably administered to locations with sufficient spatial proximity such that the adjuvant effect is adequately maintained. For example, spatial proximity is sufficient to maintain at least 50%, especially at least 75% and in particular at least 90% of the adjuvant effect seen with administration to the same location. The adjuvant effect seen with administration to the same location is defined as the level of increase observed as a result of administration of carrier-formulated mRNA and squalene emulsion adjuvant to the same location compared with administration of carrier-formulated mRNA alone. The carrier-formulated mRNA and squalene emulsion adjuvant are desirably administered to a location draining to the same lymph node, such as to the same limb, in particular to the same muscle.

Suitably carrier-formulated mRNA and squalene emulsion adjuvant are administered intramuscularly to the same muscle. In certain embodiments, the carrier-formulated mRNA and squalene emulsion adjuvant are administered to the same location.

The spatial separation of administration locations may be at least 5 mm, such as at least 1 cm.

The spatial separation of administration locations may be less than 10 cm, such as less than 5 cm apart.

When administered as separate formulations, the carrier-formulated mRNA and squalene emulsion adjuvant are desirably administered with sufficient temporal proximity such that the adjuvant effect is adequately maintained. For example, temporal proximity is sufficient to maintain at least 50%, especially at least 75% and in particular at least 90% of the adjuvant effect seen with administration at the same time. The adjuvant effect seen with administration at the same time is defined as the level of increase observed as a result of administration of carrier-formulated mRNA and squalene emulsion adjuvant at (essentially) the same time compared with administration of carrier-formulated mRNA without squalene emulsion adjuvant.

When administered as separate formulations, carrier-formulated mRNA and squalene emulsion adjuvant may be administered within 12 hours. Suitably the carrier-formulated mRNA and squalene emulsion adjuvant are administered within 6 hours, especially within 2 hours, in particular within 1 hour, such as within 30 minutes and especially within 15 minutes (e.g. within 5 minutes).

When administered as separate formulations, carrier-formulated mRNA and squalene emulsion adjuvant may be administered within 84 hours, such as within 60 hours, especially within 36 hours, in particular within 24 hours. In one embodiment the carrier-formulated mRNA and squalene emulsion adjuvant are administered within 12 to 36 hours. In another embodiment the carrier-formulated mRNA and squalene emulsion adjuvant are administered within 36 to 84 hours.

The delay between administration of the carrier-formulated mRNA and squalene emulsion adjuvant may be at least 5 seconds, such as 10 seconds, and in particular at least 30 seconds.

When administered as separate formulations, if the carrier-formulated mRNA and squalene emulsion adjuvant are administered with a delay, the carrier-formulated mRNA may be administered first and the squalene emulsion adjuvant administered second. Alternatively, the squalene emulsion adjuvant is administered first and the carrier-formulated mRNA is administered second. Appropriate temporal proximity may depend on the order of administration.

Desirably, the carrier-formulated mRNA and squalene emulsion adjuvant are administered without intentional delay (accounting for the practicalities of multiple administrations).

In addition to co-formulated or separately formulated presentations of carrier-formulated mRNA and squalene emulsion adjuvant for direct administration, the carrier-formulated mRNA and squalene emulsion adjuvant may initially be provided in various forms which facilitate manufacture, storage and distribution. For example, certain components may have limited stability in liquid form, certain components may not be amendable to drying, certain components may be incompatible when mixed (either on a short- or long-term basis). Independent of whether carrier-formulated mRNA and squalene emulsion are co-formulated at administration, they may be provided in separate containers the contents of at least some of which are subsequently combined. The skilled person will appreciate that many possibilities exist, although it is generally desirable to have a limited number of containers and limited number of required steps to prepare the final co-formulation or separate formulations for administration.

Carrier-formulated mRNA may be provided in liquid or dry (e.g. lyophilised) form. The preferred form will depend on factors such as the precise nature of the carrier-formulated mRNA, e.g. if the carrier-formulated mRNA is amenable to drying, or other components which may be present. The carrier-formulated mRNA is typically provided in liquid form.

The squalene emulsion adjuvant may be provided in liquid or dry form. The preferred form will depend on the precise nature of the squalene emulsion adjuvant, e.g. if capable of self-emulsification, and any other components present. The squalene emulsion adjuvant is typically provided in liquid form.

The invention provides a composition comprising carrier-formulated mRNA encoding an antigen and a squalene emulsion adjuvant. Typically carrier-formulated mRNA encoding an antigen and squalene emulsion adjuvant are provided as a liquid co-formulation. A liquid co-formulation enables convenient administration at the point of use.

In other embodiments the carrier-formulated mRNA encoding an antigen and squalene emulsion adjuvant are provided as a dry co-formulation, the dry co-formulation being reconstituted prior to administration. A dry co-formulation, where the components of the formulation are amendable to such presentation, may improve stability and thereby facilitate longer storage.

The carrier-formulated mRNA encoding an antigen and squalene emulsion adjuvant may be provided in separate containers. The invention therefore provides carrier-formulated mRNA encoding an antigen for use with a squalene emulsion adjuvant. Also provided is a squalene emulsion adjuvant for use with carrier-formulated mRNA encoding an antigen.

Further provided is a kit comprising:
(i) a first container comprising carrier-formulated mRNA encoding an antigen; and
(ii) a second container comprising a squalene emulsion adjuvant.

The carrier-formulated mRNA encoding an antigen may be in liquid form and the squalene emulsion adjuvant may be in liquid form. In such cases the contents of the first and second containers may be intended for combination to provide a co-formulation for administration. Alternatively, the contents of each container may be intended for separate administration as the first and second formulations.

The carrier-formulated mRNA encoding an antigen may be in dry form (e.g. lyophilised) and the squalene emulsion adjuvant may be in liquid form. In such cases the contents of the first and second containers may be intended for combination to provide a co-formulation for administration. Alternatively, the carrier-formulated mRNA encoding an antigen may be intended to be reconstituted prior to the contents of each container being used for separate administration as the first and second formulations.

The squalene emulsion adjuvant may be in dry form and the carrier-formulated mRNA encoding an antigen may be in liquid form. In such cases the contents of the first and second containers may be intended for combination to provide a co-formulation for administration.

Alternatively, the squalene emulsion adjuvant may be intended to be reconstituted prior to the contents of each container being used for separate administration as the first and second formulations.

The carrier-formulated mRNA may be in dry form (e.g. lyophilised) and the squalene emulsion adjuvant may be in dry form. In such cases the contents of the first and second containers may be intended for reconstitution and combination to provide a co-formulation for administration. Reconstitution may occur separately before combination, or the contents of one container may be reconstituted and then used to reconstitute the contents of the other container. Alternatively, the contents of the first and second containers may be intended for reconstitution prior to the contents of each container being used for separate administration as the first and second formulations.

If appropriate to the circumstances, liquid forms may be stored frozen.

The precise composition of liquid used for reconstitution will depend on both the contents of a container being reconstituted and the subsequent use of the reconstituted contents e.g. if they are intended for administration directly or may be combined with other components prior to administration. A composition (such as those containing carrier-formulated mRNA encoding an antigen or squalene emulsion adjuvant) intended for combination with other compositions prior to administration need not itself have a physiologically acceptable pH or a physiologically acceptable tonicity; a formulation intended for administration should have a physiologically acceptable pH and should have a physiologically acceptable osmolality.

The pH of a liquid preparation is adjusted in view of the components of the composition and necessary suitability for administration to the human subject. The pH of a formulation is generally at least 4, especially at least 5, in particular at least 5.5 such as at least 6. The pH of a formulation is generally 9 or less, especially 8.5 or less, in particular 8 or less, such as 7.5 or less. The pH of a formulation may be 4 to 9, especially 5 to 8.5, in particular 5.5 to 8, such as 6.5 to 7.4 (e.g. 6.5 to 7.1).

For parenteral administration, solutions should have a physiologically acceptable osmolality to avoid excessive cell distortion or lysis. A physiologically acceptable osmolality will generally mean that solutions will have an osmolality which is approximately isotonic or mildly hypertonic. Suitably the formulations for administration will have an osmolality of 250 to 750 mOsm/kg, especially 250 to 550 mOsm/kg, in particular 270 to 500 mOsm/kg, such as 270 to 400 mOsm/kg. Osmolality may be measured according to techniques known in the art, such as by the use of a commercially available osmometer, for example the Advanced® Model 2020 available from Advanced Instruments Inc. (USA).

Liquids used for reconstitution will typically be substantially aqueous, such as water for injection, phosphate buffered saline and the like. As mentioned above, the requirement for buffer and/or tonicity modifying agents will depend on both the contents of the container being reconstituted and the subsequent use of the reconstituted contents. Buffers may be selected from acetate, citrate, histidine, maleate, phosphate, succinate, tartrate and TRIS. The buffer may be a phosphate buffer such as Na/Na₂PO₄, Na/K₂PO₄ or K/K₂PO₄.

Suitably, the formulations used in the present invention have a dose volume of between 0.05 ml and 1 ml, such as between 0.1 and 0.6 ml, in particular a dose volume of 0.45 to 0.55 ml, such as 0.5 ml. The volumes of the compositions used may depend on the subject, delivery route and location, with smaller doses being given by the intradermal route or if both the carrier-formulated mRNA and squalene emulsion adjuvant are delivered to the same location. A typical human dose for administration through routes such as intramuscular, is in the region of 200 ul to 750 ml, such as 400 to 600 ul, in particular about 500 ul, such as 500 ul.

If two liquids are intended to be combined, for example for co-formulation if the carrier-formulated mRNA is in liquid form and the squalene emulsion adjuvant is in liquid form, the volume of each liquid may be the same or different. Volumes for combination will typically be in the range of 10:1 to 1:10, such as 2:1 to 1:2. Suitably the volume of each liquid will be substantially the same, such as the same. For example a 250 ul volume of carrier-formulated mRNA in liquid form may be combined with a 250 ul volume squalene emulsion adjuvant in liquid form to provide a co-formulation dose with a 500 ul volume, each of the carrier-formulated mRNA and squalene emulsion adjuvant being diluted 2-fold during the combination.

Squalene emulsion adjuvants may therefore be prepared as a concentrate with the expectation of dilution by a liquid carrier-formulated mRNA containing composition prior to administration. For example, squalene emulsion adjuvant may be prepared at double-strength with the expectation of dilution by an equal volume of carrier-formulated mRNA containing composition prior to administration.

The concentration of squalene at administration may be in the range 0.8 to 100 mg per ml, especially 1.2 to 48.4 mg per ml.

Carrier-formulated mRNA and squalene emulsion adjuvant, whether intended for co-formulation or separate formulation, may be provided in the form of various physical containers such as vials or pre-filled syringes.

In some embodiments the carrier-formulated mRNA, squalene emulsion adjuvant or kit comprising carrier-formulated mRNA and squalene emulsion adjuvant is provided in the form of a single dose. In other embodiments the carrier-formulated mRNA, squalene emulsion adjuvant or kit comprising carrier-formulated mRNA and squalene emulsion adjuvant is provided in multidose form such containing 2, 5 or 10 doses. Multidose forms, such as those comprising 10 doses, may be provided in the form of a plurality of containers with single doses of one part (e.g. the carrier-formulated mRNA) and a single container with multiple doses of the second part (e.g. squalene emulsion adjuvant) or may be provided in the form of a single container with multiple doses of one part (carrier-formulated mRNA) and a single container with multiple doses of the second part (squalene emulsion adjuvant).

It is common where liquids are to be transferred between containers, such as from a vial to a syringe, to provide ‘an overage’ which ensures that the full volume required can be conveniently transferred. The level of overage required will depend on the circumstances but excessive overage should be avoided to reduce wastage and insufficient overage may cause practical difficulties. Overages may be of the order of 20 to 100 ul per dose, such as 30 ul or 50 ul. For example, a typical 10 dose container of doubly concentrated squalene emulsion adjuvant (250 ul per dose) may contain around 2.85 to 3.25 ml of squalene emulsion adjuvant.

Stabilisers may be present. Stabilisers may be of particular relevance where multidose containers are provided as doses of the final formulation(s) may be administered to subjects over a period of time.

Carrier-formulated mRNA and squalene emulsion adjuvant in liquid form may be provided in the form of a multichamber syringe. The use of multi-chamber syringes provides a convenient method for the separate sequential administration of the carrier-formulated mRNA and squalene emulsion adjuvant. Multi-chamber syringes may be configured to provide concurrent but separate delivery of the carrier-formulated mRNA and squalene emulsion adjuvant, or they may be configured to provide sequential delivery (in either order).

In other configurations of multichambered syringes, the carrier-formulated mRNA may be provided in dry form (e.g., freeze-dried) in one chamber and reconstituted by the squalene emulsion adjuvant contained in the other chamber before administration.

Examples of multi-chamber syringes may be found in disclosures such as WO2016/172396, although a range of other configurations are possible.

Formulations are preferably sterile.

Approaches for establishing strong and lasting immunity often include repeated immunisation, i.e. boosting an immune response by administration of one or more further doses. Such further administrations may be performed with the same immunogenic compositions (homologous boosting) or with different immunogenic compositions (heterologous boosting). The present invention may be applied as part of a homologous or heterologous prime/boost regimen, as either the priming or a/the boosting immunisation.

Administration of the carrier-formulated mRNA and squalene emulsion adjuvant may therefore be part of a multi-dose administration regime. For example, the carrier-formulated mRNA and squalene emulsion adjuvant may be provided as a priming dose in a multidose regime, especially a two- or three-dose regime, in particular a two-dose regime. The carrier-formulated mRNA and squalene emulsion adjuvant may be provided as a boosting dose in a multidose regime, especially a two- or three-dose regime, such as a two-dose regime.

Priming and boosting doses may be homologous or heterologous. Consequently, the carrier-formulated mRNA and squalene emulsion adjuvant may be provided as a priming dose and boosting dose(s) in a homologous multidose regime, especially a two- or three-dose regime, in particular a two-dose regime. Alternatively, the carrier-formulated mRNA and squalene emulsion adjuvant may be provided as a priming dose or boosting dose in a heterologous multidose regime, especially a two- or three-dose regime, in particular a two-dose regime, and the boosting dose(s) may be different (e.g. a different carrier-formulated mRNA; or an alternative antigen presentation such as protein or virally vectored antigen—with or without adjuvant, such as squalene emulsion adjuvant).

The time between doses may be two weeks to six months, such as three weeks to three months. In aspects, the time between doses is at least 21 days, 28 days, 42 days, 45 days, or 60 days. In aspects, the time between doses is at least 8 weeks, 10 weeks, 12 weeks, 14 weeks, or 16 weeks. Periodic longer-term booster doses may also be provided, such as every 2 to 10 years.

The squalene emulsion adjuvant may be administered to a subject separately from carrier-formulated mRNA, or the adjuvant may be combined, either during manufacturing or extemporaneously, with carrier-formulated mRNA to provide an immunogenic composition for combined administration.

Consequently, there is provided a method for the preparation of an immunogenic composition comprising a squalene emulsion adjuvant and carrier-formulated mRNA encoding an antigen, said method comprising the steps of:
(i) preparing a squalene emulsion adjuvant;
(ii) mixing the squalene emulsion adjuvant with carrier-formulated mRNA encoding an antigen.

Also provided is a method for the preparation of an immunogenic composition comprising a squalene emulsion adjuvant and carrier-formulated mRNA encoding an antigen, said method comprising the steps of:
(i) preparing carrier-formulated mRNA encoding an antigen;
(ii) mixing the carrier-formulated mRNA encoding an antigen with squalene emulsion adjuvant.

To limit undesired degradation, squalene emulsions should generally be stored with limited exposure to oxygen e.g. in containers with limited headspace and/or by storage under nitrogen.

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in or “approximately” in relation to a numerical value x is optional and means, for example, x±10% of the given figure, such as x±5% of the given figure, in particular the given figure.

As used herein, the singular forms “a,” “an” and “the” include plural references unless the content clearly dictates otherwise.

As used herein, ng refers to nanograms, ug or pg refers to micrograms, mg refers to milligrams, mL or ml refers to milliliter, and mM refers to millimolar. Similar terms, such as um, are to be construed accordingly.

Unless specifically stated, a process comprising a step of mixing two or more components does not require any specific order of mixing. Thus components can be mixed in any order. Where there are three components then two components can be combined with each other, and then the combination may be combined with the third component, etc.

### Disclaimers

The following disclaimers may optionally be applied alone, or in combination, to any embodiment of the invention.

Suitably, the squalene emulsion adjuvant does not comprise a GLA or SLA (as specified in Carter, 2016). Desirably the squalene emulsion adjuvant does not comprise a TLR4 agonist. Desirably, the squalene emulsion adjuvant does not comprise additional immunostimulants other than tocopherol (if present).

Suitably, the squalene emulsion adjuvant does not comprise poloxamer 188.

Suitably, a formulation comprising the squalene emulsion adjuvant does not comprise a GLA or SLA (as specified in Carter, 2016). Desirably a formulation comprising the squalene emulsion adjuvant does not comprise a TLR4 agonist. Desirably, a formulation comprising the squalene emulsion adjuvant does not comprise additional immunostimulants other than tocopherol (if present).

Suitably the antigen is not a Zika virus pre-M-E antigen (prME), or an immunogenic fragment or variant thereof. Desirably the antigen is not a Zika virus antigen.

Suitably the antigen is not a respiratory syncytial virus (RSV) F antigen. Desirably the antigen is not an RSV antigen.

Suitably the antigen is not a human immunodeficiency virus (HIV) gp140 antigen.

Desirably the antigen is not an HIV antigen.

Suitably the antigen is not a Leishmania antigen.

Suitably the antigen is not a glycoprotein.

Suitably the carrier is not a LNP.

Suitably the squalene emulsion adjuvant does not comprise a cationic lipid.

Suitably a CNE carrier does not comprise DOTAP. Desirably the carrier is not a CNE.

Suitably the carrier is not a LION. Desirably the carrier does not comprise iron oxide particles, in particular the carrier does not comprise inorganic oxide particles.

Suitably the squalene emulsion adjuvant does not encapsulate or complex at least 50%, especially at least 85%, particularly at least 90% such at least 95% of the mRNA.

Suitably the squalene emulsion adjuvant is not the carrier.

Suitably administration of the carrier-formulated mRNA in conjugation with the squalene emulsion adjuvant does not significantly alter (such as less than +/−25% change, especially less than +/−10% change and in particular less than +/−5% change) the level of antigen expression observed in the absence of squalene emulsion adjuvant.

The invention is illustrated by reference to the following clauses:

Clause 1. A method of eliciting an immune response in a subject, the method comprising administering to the subject (i) carrier-formulated mRNA encoding an antigen, and (ii) a squalene emulsion adjuvant.
Clause 2. A method of adjuvanting the immune response of a subject to an antigen expressed following administration of carrier-formulated mRNA encoding the antigen, the method comprising administering to the subject a squalene emulsion adjuvant.
Clause 3. A squalene emulsion adjuvant for use in eliciting an immune response in a subject by administration with carrier-formulated mRNA encoding an antigen.
Clause 4. A carrier-formulated mRNA encoding an antigen, for use in eliciting an immune response in a subject by administration with a squalene emulsion adjuvant.
Clause 5. Use of a squalene emulsion adjuvant in the manufacture of a medicament for use in eliciting an immune response in a subject by administration with carrier-formulated mRNA encoding an antigen.
Clause 6. Use of carrier-formulated mRNA encoding an antigen in the manufacture of a medicament for use in eliciting an immune response in a subject by administration with a squalene emulsion adjuvant.
Clause 7. A kit comprising:
(i) a first container comprising carrier-formulated mRNA encoding an antigen; and
(ii) a second container comprising a squalene emulsion adjuvant.
Clause 8. The kit according to clause 7, further comprising instructions for combining a single dose of the carrier-formulated mRNA with a single dose of the squalene emulsion adjuvant to produce an immunogenic composition prior to administration of the immunogenic composition to a subject.
Clause 9. An immunogenic composition comprising: (i) carrier-formulated mRNA encoding an antigen, and (ii) a squalene emulsion adjuvant.
Clause 10. Use of (i) carrier-formulated mRNA encoding an antigen, and (ii) a squalene emulsion adjuvant, in the manufacture of a medicament.
Clause 11. The method, emulsion, carrier-formulated mRNA, use, kit or composition according to any one of clauses 1 to 10, wherein the squalene emulsion adjuvant has an average droplet size of less than 1 um, especially less than 500 nm and in particular less than 200 nm.
Clause 12. The method, emulsion, carrier-formulated mRNA, use, kit or composition according to clause 11, wherein the squalene emulsion adjuvant has an average droplet size of 50 to 200 nm, especially 120 nm to 180 nm, in particular 140 nm to 180 nm.
Clause 13. The method, emulsion, carrier-formulated mRNA, use, kit or composition according to any one of clauses 1 to 12, wherein the squalene emulsion adjuvant has a polydispersity of 0.5 or less, especially 0.3 or less, such as 0.2 or less.
Clause 14. The method, emulsion, carrier-formulated mRNA, use, kit or composition according to any one of clauses 1 to 13, wherein the squalene emulsion adjuvant surfactant is selected from poloxamer 401, poloxamer 188, polysorbate 80, sorbitan trioleate, sorbitan monooleate and polyoxyethylene 12 cetyl/stearyl ether either alone, in combination with each other or in combination with other surfactants.
Clause 15. The method, emulsion, carrier-formulated mRNA, use, kit or composition according to clause 14, wherein the squalene emulsion adjuvant surfactant is selected from polysorbate 80, sorbitan trioleate, sorbitan monooleate and polyoxyethylene 12 cetyl/stearyl ether either alone, or in combination with each other.
Clause 16. The method, emulsion, carrier-formulated mRNA, use, kit or composition according to clause 15, wherein the squalene emulsion adjuvant surfactant includes polysorbate 80.
Clause 17. The method, emulsion, carrier-formulated mRNA, use, kit or composition according to any one of clauses 1 to 16, wherein the squalene emulsion adjuvant comprises one surfactant.
Clause 18. The method, emulsion, carrier-formulated mRNA, use, kit or composition according to any one of clauses 1 to 16, wherein the squalene emulsion adjuvant comprises two surfactants.
Clause 19. The method, emulsion, carrier-formulated mRNA, use, kit or composition according to any one of clauses 1 to 16, wherein the squalene emulsion adjuvant comprises three surfactants.
Clause 20. The method, emulsion, carrier-formulated mRNA, use, kit or composition according to any one of clauses 1 to 19, wherein the amount of squalene in a single dose of the squalene emulsion adjuvant is 50 mg or less, especially 40 mg or less, in particular 30 mg or less.
Clause 21. The method, emulsion, carrier-formulated mRNA, use, kit or composition according to clause 20, wherein the amount of squalene in a single dose of the squalene emulsion adjuvant is 20 mg or less, for example 15 mg or less.
Clause 22. The method, emulsion, carrier-formulated mRNA, use, kit or composition according to any one of clauses 1 to 21, wherein the amount of squalene in a single dose of the squalene emulsion adjuvant is 0.5 mg or more, especially 1 mg or more, in particular 2 mg or more.
Clause 23. The method, emulsion, carrier-formulated mRNA, use, kit or composition according to clause 22, wherein the amount of squalene in a single dose of the squalene emulsion adjuvant is 4 mg or more, for example, 8 mg or more.
Clause 24. The method, emulsion, carrier-formulated mRNA, use, kit or composition according to any one of clauses 20 to 23, wherein the amount of squalene in a single dose of the squalene emulsion adjuvant is 2 to 4 mg.
Clause 25. The method, emulsion, carrier-formulated mRNA, use, kit or composition according to any one of clauses 20 to 23, wherein the amount of squalene in a single dose of the squalene emulsion adjuvant is 4 to 8 mg.
Clause 26. The method, emulsion, carrier-formulated mRNA, use, kit or composition according to any one of clauses 20 to 23, wherein the amount of squalene in a single dose of the squalene emulsion adjuvant is 8 to 12 mg.
Clause 27. The method, emulsion, carrier-formulated mRNA, use, kit or composition according to any one of clauses 1 to 26, wherein the weight ratio of squalene to surfactant in the squalene emulsion adjuvant is 0.73 to 6.6, especially 1 to 5, in particular 1.5 to 4.5.
Clause 28. The method, emulsion, carrier-formulated mRNA, use, kit or composition according to clause 27, wherein the weight ratio of squalene to surfactant in the squalene emulsion adjuvant is 1.5 to 3, especially 1.71 to 2.8, such as 2.2 or 2.4.
Clause 29. The method, emulsion, carrier-formulated mRNA, use, kit or composition according to clause 27, wherein the weight ratio of squalene to surfactant in the squalene emulsion adjuvant is 2.5 to 3.5, especially 3 or 3.1.
Clause 30. The method, emulsion, carrier-formulated mRNA, use, kit or composition according to clause 27, wherein the weight ratio of squalene to surfactant in the squalene emulsion adjuvant is 3 to 4.5, especially 4 or 4.3.
Clause 31. The method, emulsion, carrier-formulated mRNA, use, kit or composition according to any one of clauses 1 to 30, wherein the squalene emulsion adjuvant does not comprise tocopherol.
Clause 32. The method, emulsion, carrier-formulated mRNA, use, kit or composition according to clause 31, wherein the squalene emulsion adjuvant consists essentially of squalene, surfactant and water.
Clause 33. The method, emulsion, carrier-formulated mRNA, use, kit or composition according to clause 31, wherein the squalene emulsion adjuvant comprises squalene, polysorbate 80, sorbitan trioleate and water.
Clause 34. The method, emulsion, carrier-formulated mRNA, use, kit or composition according to clause 33, wherein the squalene emulsion adjuvant consists essentially of squalene, polysorbate 80, sorbitan trioleate and water.
Clause 35. The method, emulsion, carrier-formulated mRNA, use, kit or composition according to either clause 33 or 34, wherein squalene emulsion adjuvant comprises citrate ions e.g. 10 mM sodium citrate buffer.
Clause 36. The method, emulsion, carrier-formulated mRNA, use, kit or composition according to any one of clauses 33 to 35, wherein the weight ratio of squalene to polysorbate 80 in the squalene emulsion adjuvant is 10 to 6.6, especially 9.1 to 7.5, in particular 8.7 to 7.9 such as 8.3.
Clause 37. The method, emulsion, carrier-formulated mRNA, use, kit or composition according to any one of clauses 33 to 36, wherein the weight ratio of squalene to sorbitan trioleate in the squalene emulsion adjuvant is 10 to 6.6, especially 9.1 to 7.5, in particular 8.7 to 7.9, such as 8.3.
Clause 38. The method, emulsion, carrier-formulated mRNA, use, kit or composition according to any one of clauses 33 to 37, wherein a single dose of the squalene emulsion adjuvant comprises 0.9 to 11 mg of squalene.
Clause 39. The method, emulsion, carrier-formulated mRNA, use, kit or composition according to clause 38, wherein the single dose of the squalene emulsion adjuvant comprises 0.9 to 1.1 mg of squalene.
Clause 40. The method, emulsion, carrier-formulated mRNA, use, kit or composition according to clause 38, wherein the single dose of the squalene emulsion adjuvant comprises 1.1 to 1.4 mg of squalene.
Clause 41. The method, emulsion, carrier-formulated mRNA, use, kit or composition according to clause 38, wherein the single dose of the squalene emulsion adjuvant comprises 2.2 to 2.8 mg of squalene.
Clause 42. The method, emulsion, carrier-formulated mRNA, use, kit or composition according to clause 38, wherein the single dose of the squalene emulsion adjuvant comprises 4.5 to 5.5 mg of squalene.
Clause 43. The method, emulsion, carrier-formulated mRNA, use, kit or composition according to clause 38, wherein the single dose of the squalene emulsion adjuvant comprises 9 to 11 mg of squalene.
Clause 44. The method, emulsion, carrier-formulated mRNA, use, kit or composition according to clause 31, wherein the squalene emulsion adjuvant comprises squalene, sorbitan monooleate, polyoxyethylene cetostearyl ether and water, optionally with mannitol.
Clause 45. The method, emulsion, carrier-formulated mRNA, use, kit or composition according to clause 44, wherein the squalene emulsion adjuvant consists essentially of squalene, sorbitan monooleate, polyoxyethylene cetostearyl ether and water, optionally with mannitol.
Clause 46. The method, emulsion, carrier-formulated mRNA, use, kit or composition according to either clause 44 or 45, wherein squalene emulsion adjuvant comprises phosphate buffered saline.
Clause 47. The method, emulsion, carrier-formulated mRNA, use, kit or composition according to any one of clauses 44 to 46, wherein the weight ratio of squalene to sorbitan monooleate in the squalene emulsion adjuvant is 7.8 to 5.2, especially 7.15 to 5.85, in particular 6.8 to 6.2, such as 6.5.
Clause 48. The method, emulsion, carrier-formulated mRNA, use, kit or composition according to any one of clauses 44 to 47, wherein the weight ratio of squalene to polyoxyethylene cetostearyl ether in the squalene emulsion adjuvant is 6.2 to 4.1, especially 5.7 to 4.7, in particular 5.4 to 4.9, such as 5.2.
Clause 49. The method, emulsion, carrier-formulated mRNA, use, kit or composition according to any one of clauses 44 to 48, wherein the weight ratio of squalene to mannitol in the squalene emulsion adjuvant is 6.5 to 4.3, especially 5.9 to 4.9, in particular 5.7 to 5.1, such as 5.4.
Clause 50. The method, emulsion, carrier-formulated mRNA, use, kit or composition according to any one of clauses 44 to 49, wherein a single dose of the squalene emulsion adjuvant comprises 1.1 to 13.6 mg of squalene.
Clause 51. The method, emulsion, carrier-formulated mRNA, use, kit or composition according to clause 50, wherein the single dose of the squalene emulsion adjuvant comprises 1.1 to 1.35 mg of squalene.
Clause 52. The method, emulsion, carrier-formulated mRNA, use, kit or composition according to clause 50, wherein the single dose of the squalene emulsion adjuvant comprises 1.4 to 1.7 mg of squalene.
Clause 53. The method, emulsion, carrier-formulated mRNA, use, kit or composition according to clause 50, wherein the single dose of the squalene emulsion adjuvant comprises 2.8 to 3.4 mg of squalene.
Clause 54. The method, emulsion, carrier-formulated mRNA, use, kit or composition according to clause 50, wherein the single dose of the squalene emulsion adjuvant comprises 5.6 to 6.8 mg of squalene.
Clause 55. The method, emulsion, carrier-formulated mRNA, use, kit or composition according to clause 50, wherein the single dose of the squalene emulsion adjuvant comprises 11.2 to 13.6 mg of squalene.
Clause 56. The method, emulsion, carrier-formulated mRNA, use, kit or composition according to clause 31, wherein the squalene emulsion adjuvant comprises squalene, polysorbate 80, sorbitan trioleate and water.
Clause 57. The method, emulsion, carrier-formulated mRNA, use, kit or composition according to clause 56, wherein the squalene emulsion adjuvant consists essentially of squalene, polysorbate 80, sorbitan trioleate and water.
Clause 58. The method, emulsion, carrier-formulated mRNA, use, kit or composition according to either clause 56 or 57, wherein squalene emulsion adjuvant comprises phosphate buffered saline, such as modified phosphate buffered saline.
Clause 59. The method, emulsion, carrier-formulated mRNA, use, kit or composition according to any one of clauses 56 to 58, wherein the weight ratio of squalene to polysorbate 80 in the squalene emulsion adjuvant is 4.6 to 3.0, especially 4.2 to 3.4, in particular 4.0 to 3.6, such as 3.8.
Clause 60. The method, emulsion, carrier-formulated mRNA, use, kit or composition according to any one of clauses 56 to 59, wherein the weight ratio of squalene to sorbitan trioleate in the squalene emulsion adjuvant is 4.6 to 3.0, especially 4.2 to 3.4, in particular 4.0 to 3.6, such as 3.8.
Clause 61. The method, emulsion, carrier-formulated mRNA, use, kit or composition according to any one of clauses 56 to 60, wherein a single dose of the squalene emulsion adjuvant comprises 0.68 to 8.4 mg of squalene.
Clause 62. The method, emulsion, carrier-formulated mRNA, use, kit or composition according to clause 61, wherein the single dose of the squalene emulsion adjuvant comprises 0.68 to 0.84 mg of squalene.
Clause 63. The method, emulsion, carrier-formulated mRNA, use, kit or composition according to clause 61, wherein the single dose of the squalene emulsion adjuvant comprises 0.85 to 1.1 mg of squalene.
Clause 64. The method, emulsion, carrier-formulated mRNA, use, kit or composition according to clause 61, wherein the single dose of the squalene emulsion adjuvant comprises 1.7 to 2.1 mg of squalene.
Clause 65. The method, emulsion, carrier-formulated mRNA, use, kit or composition according to clause 61, wherein the single dose of the squalene emulsion adjuvant comprises 3.4 to 4.2 mg of squalene.
Clause 66. The method, emulsion, carrier-formulated mRNA, use, kit or composition according to clause 61, wherein the single dose of the squalene emulsion adjuvant comprises 6.8 to 8.4 mg of squalene.
Clause 67. The method, emulsion, carrier-formulated mRNA, use, kit or composition according to clause 31, wherein the squalene emulsion adjuvant comprises squalene, phosphatidyl choline, poloxamer 188 and water, optionally with glycerol.
Clause 68. The method, emulsion, carrier-formulated mRNA, use, kit or composition according to clause 67, wherein the squalene emulsion adjuvant consists essentially of squalene, phosphatidyl choline, poloxamer 188 and water, optionally with glycerol.
Clause 69. The method, emulsion, carrier-formulated mRNA, use, kit or composition according to either clause 67 or 68, wherein the squalene emulsion adjuvant comprises ammonium phosphate buffer and/or glycerol.
Clause 70. The method, emulsion, carrier-formulated mRNA, use, kit or composition according to any one of clauses 67 to 69, wherein the weight ratio of squalene to phosphatidyl choline in the squalene emulsion adjuvant is 2.52 to 3.8, especially 2.85 to 3.5, in particular 3 to 3.3, such as 3.15
Clause 71. The method, emulsion, carrier-formulated mRNA, use, kit or composition according to any one of clauses 67 to 70, wherein the weight ratio of squalene to poloxamer 188 in the squalene emulsion adjuvant is 55 to 83, especially 62 to 76, in particular 65.5 to 72.5, such as 69.
Clause 72. The method, emulsion, carrier-formulated mRNA, use, kit or composition according to any one of clauses 67 to 71, wherein a single dose of the squalene emulsion adjuvant comprises 0.77 to 9.5 mg of squalene.
Clause 73. The method, emulsion, carrier-formulated mRNA, use, kit or composition according to clause 72, wherein the single dose of the squalene emulsion adjuvant comprises 0.77 to 0.95 mg of squalene.
Clause 74. The method, emulsion, carrier-formulated mRNA, use, kit or composition according to clause 72, wherein the single dose of the squalene emulsion adjuvant comprises 0.96 to 1.2 mg of squalene.
Clause 75. The method, emulsion, carrier-formulated mRNA, use, kit or composition according to clause 72, wherein the single dose of the squalene emulsion adjuvant comprises 1.9 to 2.4 mg of squalene.
Clause 76. The method, emulsion, carrier-formulated mRNA, use, kit or composition according to clause 72, wherein the single dose of the squalene emulsion adjuvant comprises 3.8 to 4.8 mg of squalene.
Clause 77. The method, emulsion, carrier-formulated mRNA, use, kit or composition according to clause 72, wherein the single dose of the squalene emulsion adjuvant comprises 7.7 to 9.5 mg of squalene.
Clause 78. The method, emulsion, carrier-formulated mRNA, use, kit or composition according to any one of clauses 1 to 30, wherein the squalene emulsion adjuvant comprises tocopherol.
Clause 79. The method, emulsion, carrier-formulated mRNA, use, kit or composition according to clause 78, wherein the tocopherol is alpha-tocopherol.
Clause 80. The method, emulsion, carrier-formulated mRNA, use, kit or composition according to clause 79, wherein the tocopherol is D/L-alpha-tocopherol.
Clause 81. The method, emulsion, carrier-formulated mRNA, use, kit or composition according to any one of clauses 78 to 80, wherein the squalene emulsion adjuvant consists essentially of squalene, tocopherol, surfactant and water.
Clause 82. The method, emulsion, carrier-formulated mRNA, use, kit or composition according to any one of clauses 78 to 81, wherein the weight ratio of squalene to tocopherol in the squalene emulsion adjuvant is 20 or less, especially 10 or less.
Clause 83. The method, emulsion, carrier-formulated mRNA, use, kit or composition according to any one of clauses 78 to 82, wherein the weight ratio of squalene to tocopherol in the squalene emulsion adjuvant is 0.1 or more.
Clause 84. The method, emulsion, carrier-formulated mRNA, use, kit or composition according to any one of clauses 78 to 83, wherein the weight ratio of squalene to tocopherol in the squalene emulsion adjuvant is 0.2 to 5.
Clause 85. The method, emulsion, carrier-formulated mRNA, use, kit or composition according to clause 84, wherein the weight ratio of squalene to tocopherol in the squalene emulsion adjuvant is 0.72 to 1.136, especially 0.8 to 1, in particular 0.85 to 0.95, such as 0.9.
Clause 86. The method, emulsion, carrier-formulated mRNA, use, kit or composition according to clause 84, wherein the weight ratio of squalene to tocopherol in the squalene emulsion adjuvant is 3.4 to 4.6, especially 3.6 to 4.4, in particular 3.8 to 4.2, such as 4.
Clause 87. The method, emulsion, carrier-formulated mRNA, use, kit or composition according to one of clauses 78 to 86, wherein the squalene emulsion adjuvant comprises squalene, tocopherol, polysorbate 80 and water.
Clause 88. The method, emulsion, carrier-formulated mRNA, use, kit or composition according to clause 87, wherein the squalene emulsion adjuvant consists essentially of squalene, tocopherol, polysorbate 80 and water.
Clause 89. The method, emulsion, carrier-formulated mRNA, use, kit or composition according to either clause 87 or 88, wherein squalene emulsion adjuvant comprises a phosphate buffered saline, such as modified phosphate buffered saline.
Clause 90. The method, emulsion, carrier-formulated mRNA, use, kit or composition according to any one of clauses 87 to 89, wherein the weight ratio of squalene to tocopherol in the squalene emulsion adjuvant is 0.5 to 1.5, especially 0.6 to 1.35, in particular 0.7 to 1.1, such as 0.85 to 0.95 e.g. 0.9.
Clause 91. The method, emulsion, carrier-formulated mRNA, use, kit or composition according to any one of clauses 87 to 90, wherein the weight ratio of squalene to polysorbate 80 in the squalene emulsion adjuvant is 1.2 to 3.6, especially 1.46 to 3.3, in particular 1.9 to 2.5 such as 2.1 to 2.3 e.g. 2.2.
Clause 92. The method, emulsion, carrier-formulated mRNA, use, kit or composition according to any one of clauses 87 to 91, wherein a single dose of the squalene emulsion adjuvant comprises 0.9 to 12.1 mg of squalene.
Clause 93. The method, emulsion, carrier-formulated mRNA, use, kit or composition according to clause 92, wherein the single dose of the squalene emulsion adjuvant comprises 0.9 to 1.3 mg of squalene, typically with 1 to 1.4 mg tocopherol and 0.43 to 0.57 mg polysorbate 80.
Clause 94. The method, emulsion, carrier-formulated mRNA, use, kit or composition according to clause 92, wherein the single dose of the squalene emulsion adjuvant comprises 1.2 to 1.6 mg of squalene, typically with 1.3 to 1.7 mg tocopherol and 0.54 to 0.71 mg polysorbate 80.
Clause 95. The method, emulsion, carrier-formulated mRNA, use, kit or composition according to clause 92, wherein the single dose of the squalene emulsion adjuvant comprises 2.4 to 3 mg of squalene, typically with 2.6 to 3.4 mg tocopherol and 1 to 1.5 mg polysorbate 80.
Clause 96. The method, emulsion, carrier-formulated mRNA, use, kit or composition according to clause 92, wherein the single dose of the squalene emulsion adjuvant comprises 4.8 to 6.1 mg of squalene, typically with 5.3 to 6.8 mg tocopherol and 2.1 to 2.9 mg polysorbate 80.
Clause 97. The method, emulsion, carrier-formulated mRNA, use, kit or composition according to clause 92, wherein the single dose of the squalene emulsion adjuvant comprises 9.7 to 12.1 mg of squalene, typically with 10.6 to 13.6 mg tocopherol and 4.3 to 5.7 mg polysorbate 80.
Clause 98. The method, emulsion, carrier-formulated mRNA, use, kit or composition according to any one of clauses 87 to 89, wherein the weight ratio of squalene to tocopherol in the squalene emulsion adjuvant is 2.6 to 4.5, especially 2.8 to 4.3, in particular 3.25 to 4, such as 3.4 to 3.8 e.g. 3.6.
Clause 99. The method, emulsion, carrier-formulated mRNA, use, kit or composition according to any one of clauses 87 to 89 or 98, wherein the weight ratio of squalene to polysorbate 80 in the squalene emulsion adjuvant is 11.3 to 2.5, especially 1.56 to 2.3, in particular 1.75 to 2.15 such as 1.85 to 2 e.g. 1.94.
Clause 100. The method, emulsion, carrier-formulated mRNA, use, kit or composition according to any one of clauses 87 to 89, 98 or 99, wherein a single dose of the squalene emulsion adjuvant comprises 1.1 to 14.3 mg of squalene.
Clause 101. The method, emulsion, carrier-formulated mRNA, use, kit or composition according to clause 100, wherein the single dose of the squalene emulsion adjuvant comprises 1.1 to 1.5 mg of squalene.
Clause 102. The method, emulsion, carrier-formulated mRNA, use, kit or composition according to clause 100, wherein the single dose of the squalene emulsion adjuvant comprises 1.4 to 1.8 mg of squalene.
Clause 103. The method, emulsion, carrier-formulated mRNA, use, kit or composition according to clause 100, wherein the single dose of the squalene emulsion adjuvant comprises 2.9 to 3.6 mg of squalene.
Clause 104. The method, emulsion, carrier-formulated mRNA, use, kit or composition according to clause 100, wherein the single dose of the squalene emulsion adjuvant comprises 5.8 to 7.2 mg of squalene.
Clause 105. The method, emulsion, carrier-formulated mRNA, use, kit or composition according to clause 100, wherein the single dose of the squalene emulsion adjuvant comprises 11.7 to 14.3 mg of squalene.
Clause 106. The method, emulsion, carrier-formulated mRNA, use, kit or composition according to any one of clauses 78 to 83, wherein the squalene emulsion adjuvant comprises squalene, tocopherol, phosphatidyl choline, poloxamer 188 and water, optionally with glycerol.
Clause 107. The method, emulsion, carrier-formulated mRNA, use, kit or composition according to clause 106, wherein the squalene emulsion adjuvant consists essentially of squalene, tocopherol, phosphatidyl choline, poloxamer 188 and water, optionally with glycerol.
Clause 108. The method, emulsion, carrier-formulated mRNA, use, kit or composition according to either clause 106 or 107, wherein squalene emulsion adjuvant comprises ammonium phosphate buffer and/or glycerol.
Clause 109. The method, emulsion, carrier-formulated mRNA, use, kit or composition according to any one of clauses 106 to 108, wherein the weight ratio of squalene to tocopherol in the squalene emulsion adjuvant is at least 50, especially 137 to 207, in particular 154 to 190, such as 163 to 181, for example 172.
Clause 110. The method, emulsion, carrier-formulated mRNA, use, kit or composition according to any one of clauses 106 to 109, wherein the weight ratio of squalene to phosphatidyl choline in the squalene emulsion adjuvant is 2.52 to 3.8, especially 2.85 to 3.5, in particular 3 to 3.3, such as 3.15
Clause 111. The method, emulsion, carrier-formulated mRNA, use, kit or composition according to any one of clauses 106 to 110, wherein the weight ratio of squalene to poloxamer 188 in the squalene emulsion adjuvant is 55 to 83, especially 62 to 76, in particular 65.5 to 72.5, such as 69.
Clause 112. The method, emulsion, carrier-formulated mRNA, use, kit or composition according to any one of clauses 106 to 111, wherein a single dose of the squalene emulsion adjuvant comprises 0.77 to 9.5 mg of squalene.
Clause 113. The method, emulsion, carrier-formulated mRNA, use, kit or composition according to clause 112, wherein the single dose of the squalene emulsion adjuvant comprises 0.77 to 0.95 mg of squalene.
Clause 114. The method, emulsion, carrier-formulated mRNA, use, kit or composition according to clause 112, wherein the single dose of the squalene emulsion adjuvant comprises 0.96 to 1.2 mg of squalene.
Clause 115. The method, emulsion, carrier-formulated mRNA, use, kit or composition according to clause 112, wherein the single dose of the squalene emulsion adjuvant comprises 1.9 to 2.4 mg of squalene.
Clause 116. The method, emulsion, carrier-formulated mRNA, use, kit or composition according to clause 112, wherein the single dose of the squalene emulsion adjuvant comprises 3.8 to 4.8 mg of squalene.
Clause 117. The method, emulsion, carrier-formulated mRNA, use, kit or composition according to clause 112, wherein the single dose of the squalene emulsion adjuvant comprises 7.7 to 9.5 mg of squalene.
Clause 118. The method, emulsion, carrier-formulated mRNA, use, kit or composition according to any one of clauses 1 to 117, for administration to a human subject.
Clause 119. The method, emulsion, carrier-formulated mRNA, use, kit or composition according to clause 118, for administration to a human child.
Clause 120. The method, emulsion, carrier-formulated mRNA, use, kit or composition according to clause 118, for administration to a human adult (aged 18-59).
Clause 121. The method, emulsion, carrier-formulated mRNA, use, kit or composition according to clause 118, for administration to an older human (aged 60 or greater).
Clause 122. The method, emulsion, carrier-formulated mRNA, use, kit or composition according to any one of clauses 1 to 121, wherein the antigen comprises at least one B or T cell epitope.
Clause 123. The method, emulsion, carrier-formulated mRNA, use, kit or composition according to clause 122, wherein the antigen comprises B and T cell epitopes.
Clause 124. The method, emulsion, carrier-formulated mRNA, use, kit or composition according to either clause 122 or 123, wherein the antigen contains 3000 residues or fewer, especially 2000 residues or fewer, in particular 1500 residues or fewer.
Clause 125. The method, emulsion, carrier-formulated mRNA, use, kit or composition according to clause 124, wherein the antigen contains 1000 residues or fewer, 800 residues or fewer, 600 residues or fewer, 400 residues or fewer or 200 residues or fewer.
Clause 126. The method, emulsion, carrier-formulated mRNA, use, kit or composition according to any one of clauses 122 to 125, wherein the antigen contains 50 residues or more, especially 100 residues or more.
Clause 127. The method, emulsion, carrier-formulated mRNA, use, kit or composition according to any one of clauses 122 to 126, wherein the antigen is derived from a pathogen, such as a bacterium, virus or parasite.
Clause 128. The method, emulsion, carrier-formulated mRNA, use, kit or composition according to clause 127, wherein the pathogen is a bacterium which is pathogenic in humans.
Clause 129. The method, emulsion, carrier-formulated mRNA, use, kit or composition according to clause 127, wherein the pathogen is a virus which is pathogenic in humans.
Clause 130. The method, emulsion, carrier-formulated mRNA, use, kit or composition according to clause 127, wherein the pathogen is a parasite which is pathogenic in humans.
Clause 131. The method, emulsion, carrier-formulated mRNA, use, kit or composition according to any one of clauses 122 to 126, wherein the antigen is a cancer antigen, such as a tumour antigen and/or a neoantigen.
Clause 132. The method, emulsion, carrier-formulated mRNA, use, kit or composition according to clause 131, wherein the antigen is a human cancer antigen, such as a human tumour antigen and/or a human neoantigen.
Clause 133. The method, emulsion, carrier-formulated mRNA, use, kit or composition according to any one of clauses 122 to 127 or 129 wherein the antigen is derived from at least one coronavirus.
Clause 134. The method, emulsion, carrier-formulated mRNA, use, kit or composition according to clause 133, wherein the antigen is derived from one coronavirus.
Clause 135. The method, emulsion, carrier-formulated mRNA, use, kit or composition according to clause 133, wherein the antigen is derived from more than one coronavirus.
Clause 136. The method, emulsion, carrier-formulated mRNA, use, kit or composition according to either clause 134 or 135 wherein the coronavirus antigen is derived from SARS-CoV-2.
Clause 137. The method, emulsion, carrier-formulated mRNA, use, kit or composition according to clause 136 wherein the SARS-CoV-2 antigen is a SARS-CoV-2 S protein.
Clause 138. The method, emulsion, carrier-formulated mRNA, use, kit or composition according to clause 137, wherein the SARS-CoV-2 S protein is a pre-fusion stabilised S protein.
Clause 139. The method, emulsion, carrier-formulated mRNA, use, kit or composition according to either clause 137 or 138, wherein the SARS-CoV-2 S protein comprises a trimerization motif.
Clause 140. The method, emulsion, carrier-formulated mRNA, use, kit or composition according to any one of clauses 137 to 139, wherein the SARS-CoV-2 S protein comprises an amino acid sequence having at least 90% identity to the amino acid sequence set forth in SEQ ID NO:1.
Clause 141. The method, emulsion, carrier-formulated mRNA, use, kit or composition according to clause 140, wherein the SARS-CoV-2 S protein consists of an amino acid sequence having at least 90% identity to the amino acid sequence set forth in SEQ ID NO:1.
Clause 142. The method, emulsion, carrier-formulated mRNA, use, kit or composition according to clause 140, wherein the SARS-CoV-2 S protein comprises an amino acid sequence having at least 95% identity to the amino acid sequence set forth in SEQ ID NO:1.
Clause 143. The method, emulsion, carrier-formulated mRNA, use, kit or composition according to clause 142, wherein the SARS-CoV-2 S protein consists of an amino acid sequence having at least 95% identity to the amino acid sequence set forth in SEQ ID NO:1.
Clause 144. The method, emulsion, carrier-formulated mRNA, use, kit or composition according to clause 140, wherein the SARS-CoV-2 S protein comprises an amino acid sequence having at least 98% identity to the amino acid sequence set forth in SEQ ID NO:1.
Clause 145. The method, emulsion, carrier-formulated mRNA, use, kit or composition according to clause 144, wherein the SARS-CoV-2 S protein consists of an amino acid sequence having at least 98% identity to the amino acid sequence set forth in SEQ ID NO:1.
Clause 146. The method, emulsion, carrier-formulated mRNA, use, kit or composition according to clause 140, wherein the SARS-CoV-2 S protein comprises an amino acid sequence having at least 99% identity to the amino acid sequence set forth in SEQ ID NO:1.
Clause 147. The method, emulsion, carrier-formulated mRNA, use, kit or composition according to clause 146, wherein the SARS-CoV-2 S protein consists of an amino acid sequence having at least 99% identity to the amino acid sequence set forth in SEQ ID NO:1.
Clause 148. The method, emulsion, carrier-formulated mRNA, use, kit or composition according to clause 140, wherein the SARS-CoV-2 S protein comprises the amino acid sequence set forth in SEQ ID NO:1.
Clause 149. The method, emulsion, carrier-formulated mRNA, use, kit or composition according to clause 148, wherein the SARS-CoV-2 S protein consists of the amino acid sequence set forth in SEQ ID NO:1.
Clause 150. The method, emulsion, carrier-formulated mRNA, use, kit or composition according to any one of clauses 137 to 139, wherein the SARS-CoV-2 S protein comprises an amino acid sequence having at least 90% identity to the amino acid sequence set forth in SEQ ID NO:2.
Clause 151. The method, emulsion, carrier-formulated mRNA, use, kit or composition according to clause 150, wherein the SARS-CoV-2 S protein consists of an amino acid sequence having at least 90% identity to the amino acid sequence set forth in SEQ ID NO:2.
Clause 152. The method, emulsion, carrier-formulated mRNA, use, kit or composition according to clause 150, wherein the SARS-CoV-2 S protein comprises an amino acid sequence having at least 95% identity to the amino acid sequence set forth in SEQ ID NO:2.
Clause 153. The method, emulsion, carrier-formulated mRNA, use, kit or composition according to clause 152, wherein the SARS-CoV-2 S protein consists of an amino acid sequence having at least 95% identity to the amino acid sequence set forth in SEQ ID NO:2.
Clause 154. The method, emulsion, carrier-formulated mRNA, use, kit or composition according to clause 150, wherein the SARS-CoV-2 S protein comprises an amino acid sequence having at least 98% identity to the amino acid sequence set forth in SEQ ID NO:2.
Clause 155. The method, emulsion, carrier-formulated mRNA, use, kit or composition according to clause 154, wherein the SARS-CoV-2 S protein consists of an amino acid sequence having at least 98% identity to the amino acid sequence set forth in SEQ ID NO:2.
Clause 156. The method, emulsion, carrier-formulated mRNA, use, kit or composition according to clause 150, wherein the SARS-CoV-2 S protein comprises an amino acid sequence having at least 99% identity to the amino acid sequence set forth in SEQ ID NO:2.
Clause 157. The method, emulsion, carrier-formulated mRNA, use, kit or composition according to clause 156, wherein the SARS-CoV-2 S protein consists of an amino acid sequence having at least 99% identity to the amino acid sequence set forth in SEQ ID NO:2.
Clause 158. The method, emulsion, carrier-formulated mRNA, use, kit or composition according to clause 150, wherein the SARS-CoV-2 S protein comprises the amino acid sequence set forth in SEQ ID NO:2.
Clause 159. The method, emulsion, carrier-formulated mRNA, use, kit or composition according to clause 158, wherein the SARS-CoV-2 S protein consists of the amino acid sequence set forth in SEQ ID NO:2.
Clause 160. The method, emulsion, carrier-formulated mRNA, use, kit or composition according to any one of clauses 137 to 139, wherein the SARS-CoV-2 S protein comprises an amino acid sequence having at least 90% identity to the amino acid sequence set forth in SEQ ID NO:3.
Clause 161. The method, emulsion, carrier-formulated mRNA, use, kit or composition according to clause 160, wherein the SARS-CoV-2 S protein consists of an amino acid sequence having at least 90% identity to the amino acid sequence set forth in SEQ ID NO:3.
Clause 162. The method, emulsion, carrier-formulated mRNA, use, kit or composition according to clause 160, wherein the SARS-CoV-2 S protein comprises an amino acid sequence having at least 95% identity to the amino acid sequence set forth in SEQ ID NO:3.
Clause 163. The method, emulsion, carrier-formulated mRNA, use, kit or composition according to clause 162, wherein the SARS-CoV-2 S protein consists of an amino acid sequence having at least 95% identity to the amino acid sequence set forth in SEQ ID NO:3.
Clause 164. The method, emulsion, carrier-formulated mRNA, use, kit or composition according to clause 160, wherein the SARS-CoV-2 S protein comprises an amino acid sequence having at least 98% identity to the amino acid sequence set forth in SEQ ID NO:3.
Clause 165. The method, emulsion, carrier-formulated mRNA, use, kit or composition according to clause 164, wherein the SARS-CoV-2 S protein consists of an amino acid sequence having at least 98% identity to the amino acid sequence set forth in SEQ ID NO:3.
Clause 166. The method, emulsion, carrier-formulated mRNA, use, kit or composition according to clause 160, wherein the SARS-CoV-2 S protein comprises an amino acid sequence having at least 99% identity to the amino acid sequence set forth in SEQ ID NO:3.
Clause 167. The method, emulsion, carrier-formulated mRNA, use, kit or composition according to clause 166, wherein the SARS-CoV-2 S protein consists of an amino acid sequence having at least 99% identity to the amino acid sequence set forth in SEQ ID NO:3.
Clause 168. The method, emulsion, carrier-formulated mRNA, use, kit or composition according to clause 160, wherein the SARS-CoV-2 S protein comprises the amino acid sequence set forth in SEQ ID NO:3.
Clause 169. The method, emulsion, carrier-formulated mRNA, use, kit or composition according to clause 168, wherein the SARS-CoV-2 S protein consists of the amino acid sequence set forth in SEQ ID NO:3.
Clause 170. The method, emulsion, carrier-formulated mRNA, use, kit or composition according to any one of clauses 137 to 139, wherein the SARS-CoV-2 S protein comprises an amino acid sequence having at least 90% identity to the amino acid sequence set forth in SEQ ID NO:4.
Clause 171. The method, emulsion, carrier-formulated mRNA, use, kit or composition according to clause 170, wherein the SARS-CoV-2 S protein consists of an amino acid sequence having at least 90% identity to the amino acid sequence set forth in SEQ ID NO:4.
Clause 172. The method, emulsion, carrier-formulated mRNA, use, kit or composition according to clause 170, wherein the SARS-CoV-2 S protein comprises an amino acid sequence having at least 95% identity to the amino acid sequence set forth in SEQ ID NO:4.
Clause 173. The method, emulsion, carrier-formulated mRNA, use, kit or composition according to clause 172, wherein the SARS-CoV-2 S protein consists of an amino acid sequence having at least 95% identity to the amino acid sequence set forth in SEQ ID NO:4.
Clause 174. The method, emulsion, carrier-formulated mRNA, use, kit or composition according to clause 170, wherein the SARS-CoV-2 S protein comprises an amino acid sequence having at least 98% identity to the amino acid sequence set forth in SEQ ID NO:4.
Clause 175. The method, emulsion, carrier-formulated mRNA, use, kit or composition according to clause 174, wherein the SARS-CoV-2 S protein consists of an amino acid sequence having at least 98% identity to the amino acid sequence set forth in SEQ ID NO:4.
Clause 176. The method, emulsion, carrier-formulated mRNA, use, kit or composition according to clause 170, wherein the SARS-CoV-2 S protein comprises an amino acid sequence having at least 99% identity to the amino acid sequence set forth in SEQ ID NO:4.
Clause 177. The method, emulsion, carrier-formulated mRNA, use, kit or composition according to clause 176, wherein the SARS-CoV-2 S protein consists of an amino acid sequence having at least 99% identity to the amino acid sequence set forth in SEQ ID NO:4.
Clause 178. The method, emulsion, carrier-formulated mRNA, use, kit or composition according to clause 170, wherein the SARS-CoV-2 S protein comprises the amino acid sequence set forth in SEQ ID NO:4.
Clause 179. The method, emulsion, carrier-formulated mRNA, use, kit or composition according to clause 178, wherein the SARS-CoV-2 S protein consists of the amino acid sequence set forth in SEQ ID NO:4.
Clause 180. The method, emulsion, carrier-formulated mRNA, use, kit or composition according any one of clauses 137 to 179, wherein the encoded SARS-CoV-2 S protein is 1800 residues or fewer in length, especially 1500 residues or fewer, in particular 1400 residues or fewer, such as 1300 residues or fewer.
Clause 181. The method, emulsion, carrier-formulated mRNA, use, kit or composition according to any one of clauses 1 to 180, wherein the mRNA comprises a 5′ cap.
Clause 182. The method, emulsion, carrier-formulated mRNA, use, kit or composition according to any one of clauses 1 to 181, wherein the mRNA comprises a poly A tail.
Clause 183. The method, emulsion, carrier-formulated mRNA, use, kit or composition according to any one of clauses 1 to 182, wherein the mRNA comprises a 5′ untranslated region (UTR).
Clause 184. The method, emulsion, carrier-formulated mRNA, use, kit or composition according to any one of clauses 1 to 183, wherein the mRNA comprises a 3′ UTR.
Clause 185. The method, emulsion, carrier-formulated mRNA, use, kit or composition according to any one of clauses 1 to 184, where in the mRNA comprises at least one chemical modification.
Clause 186. The method, emulsion, carrier-formulated mRNA, use, kit or composition according to clause 185, wherein the chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.
Clause 187. The method, emulsion, carrier-formulated mRNA, use, kit or composition according to clause 185, wherein the chemical modification is in the 5-position of the uracil.
Clause 188. The method, emulsion, carrier-formulated mRNA, use, kit or composition according to clause 185, where all of the uracil in the open reading frame encoding the antigen have a chemical modification, such as all of the uracil in mRNA have a chemical modification. Clause The method, emulsion, carrier-formulated mRNA, use, kit or composition according to any one of clauses 185 to 188, wherein the chemical modification is N1-methylpseudouridine.
Clause 190. The method, emulsion, carrier-formulated mRNA, use, kit or composition according to any one of clauses 185 to 188, wherein the chemical modification is N1-ethylpseudouridine.
Clause 191. The method, emulsion, carrier-formulated mRNA, use, kit or composition according to any one of clauses 1 to 184, where in the mRNA is not chemically modified.
Clause 192. The method, emulsion, carrier-formulated mRNA, use, kit or composition according to any one of clauses 1 to 191, wherein each mRNA is non-replicating.
Clause 193. The method, emulsion, carrier-formulated mRNA, use, kit or composition according to clause 192, wherein the mRNA has the configuration 5′-cap-5′-UTR-[antigen]-3′-UTR-PolyA.
Clause 194. The method, emulsion, carrier-formulated mRNA, use, kit or composition according to either clause 192 or 193, wherein the mRNA contains 10000 bases or fewer, especially 8000 bases or fewer, in particular 5000 bases or fewer.
Clause 195. The method, emulsion, carrier-formulated mRNA, use, kit or composition according to any one of clauses 1 to 191, wherein the mRNA is self-replicating.
Clause 196. The method, emulsion, carrier-formulated mRNA, use, kit or composition according to clause 195 wherein the self-replicating RNA molecule encodes (i) a RNA-dependent RNA polymerase which can transcribe RNA from the self-replicating RNA molecule and (ii) an immunogen.
Clause 197. The method, emulsion, carrier-formulated mRNA, use, kit or composition according to clause 196 wherein the RNA molecule comprises two open reading frames, the first of which encodes an alphavirus replicase and the second of which encodes the immunogen.
Clause 198. The method, emulsion, carrier-formulated mRNA, use, kit or composition according to any one of clauses 195 to 197, wherein the mRNA has the configuration 5′-cap-5′UTR-non-structural proteins (NSP) 1-4-subgenomic promoter-[antigen]-3′UTR-polyA.
Clause 199. The method, emulsion, carrier-formulated mRNA, use, kit or composition according to any one of clauses 195 to 198, wherein the mRNA contains 25000 bases or fewer especially 20000 bases or fewer, in particular 15000 bases or fewer.
Clause 200. The method, emulsion, carrier-formulated mRNA, use, kit or composition according to clause 199, wherein the RNA molecule is 9000 to 12000 nucleotides long.
Clause 201. The method, emulsion, carrier-formulated mRNA, use, kit or composition according to any one of clauses 1 to 200, wherein a single dose of mRNA is 0.001 to 1000 ug, especially 1 to 500 ug, in particular 10 to 250 ug.
Clause 202. The method, emulsion, carrier-formulated mRNA, use, kit or composition according to clause 201, wherein a single dose of mRNA is 0.001 to 75 ug.
Clause 203. The method, emulsion, carrier-formulated mRNA, use, kit or composition according to clause 201, wherein a single dose of mRNA is 1 to 75 ug.
Clause 204. The method, emulsion, carrier-formulated mRNA, use, kit or composition according to clause 201, wherein a single dose of mRNA is 25 to 250 ug.
Clause 205. The method, emulsion, carrier-formulated mRNA, use, kit or composition according to clause 201, wherein a single dose of mRNA is 250 to 1000 ug.
Clause 206. The method, emulsion, carrier-formulated mRNA, use, kit or composition according to any one of clauses 1 to 205, wherein the carrier is a lipid nanoparticle (LNP).
Clause 207. The method, emulsion, carrier-formulated mRNA, use, kit or composition according to clause 206, wherein the LNP comprise a PEG-modified lipid, a non-cationic lipid, a sterol, and a non-ionisable cationic lipid.
Clause 208. The method, emulsion, carrier-formulated mRNA, use, kit or composition according to clause 206, wherein the LNP comprise a PEG-modified lipid, a non-cationic lipid, a sterol, and an ionisable cationic lipid.
Clause 209. The method, emulsion, carrier-formulated mRNA, use, kit or composition according to either clause 207 or 208, wherein the non-cationic lipid is a neutral lipid, such as 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE) or sphingomyelin (SM).
Clause 210. The method, emulsion, carrier-formulated mRNA, use, kit or composition according to any one of clauses 207 to 209, wherein the sterol is cholesterol.
Clause 211. The method, emulsion, carrier-formulated mRNA, use, kit or composition according to any one of clauses 208 to 210, wherein the LNP comprise a PEG-modified lipid at around 0.5 to 15 molar %, a non-cationic lipid at around 5 to 25 molar %, a sterol at around 25 to 55 molar % and an ionisable cationic lipid at around 20 to 60 molar %.
Clause 212. The method, emulsion, carrier-formulated mRNA, use, kit or composition according to any one of clauses 206 to 211, wherein the LNP are 50 to 200 um in diameter.
Clause 213. The method, emulsion, carrier-formulated mRNA, use, kit or composition according to any one of clauses 206 to 212, wherein the LNP have a polydispersity of 0.4 or less, such as 0.3 or less.
Clause 214. The method, emulsion, carrier-formulated mRNA, use, kit or composition according to any one of clauses 206 to 213, where the ratio of nucleotide (N) to phospholipid (P) is in the range of 1N:1P to 20N:1P, 1N:1P to 10N:1P, 2N:1P to 8N:1P, 2N:1P to 6N:1P or 3N:1P to 5N:1P.
Clause 215. The method, emulsion, carrier-formulated mRNA, use, kit or composition according to any one of clauses 206 to 214, wherein at least half of the RNA is encapsulated in the LNP, suitably at least 85%, especially at least 95%, such as all of it.
Clause 216. The method, emulsion, carrier-formulated mRNA, use, kit or composition according to any one of clauses 1 to 205, wherein the carrier is a cationic nanoemulsion (CNE).
Clause 217. The method, emulsion, carrier-formulated mRNA, use, kit or composition according to clause 216, wherein the CNE is an oil-in-water emulsion of DOTAP and squalene stabilised with polysorbate 80 and/or sorbitan trioleate.
Clause 218. The method, emulsion, carrier-formulated mRNA, use, kit or composition according to either clause 216 or 217, wherein the CNE droplets are 50 to 200 um in diameter.
Clause 219. The method, emulsion, carrier-formulated mRNA, use, kit or composition according to any one of clauses 216 to 218, wherein the CNE droplets have a polydispersity of 0.4 or less, such as 0.3 or less.
Clause 220. The method, emulsion, carrier-formulated mRNA, use, kit or composition according to any one of clauses 216 to 219, wherein at least half of the RNA is complexed with the CNE, suitably at least 85%, especially at least 95%, such as all of it.
Clause 221. The method, emulsion, carrier-formulated mRNA, use, kit or composition according to any one of clauses 1 to 205, wherein the carrier is a lipidoid-coated iron oxide nanoparticle (LION).
Clause 222. The method, emulsion, carrier-formulated mRNA, use, kit or composition according to clause 221, wherein each LION comprises 200 to 5000, such as 500 to 2000, in particular about 1000 lipid and/or lipidoid molecules.
Clause 223. The method, emulsion, carrier-formulated mRNA, use, kit or composition according to either clause 221 or 222, wherein the LION comprises lipidoids comprising alkyl tails of 12 to 14 carbons in length, especially lipidoid C14-200.
Clause 224. The method, emulsion, carrier-formulated mRNA, use, kit or composition according to any one of clauses 221 to 223, wherein the LION are 20 to 200 um in diameter, such as 50 to 100 nm in diameter.
Clause 225. The method, emulsion, carrier-formulated mRNA, use, kit or composition according to any one of clauses 221 to 224, wherein the lipid/lipidoid to mRNA weight ratio is about 1:1 to 10:1, especially about 5:1.
Clause 226. The method, emulsion, carrier-formulated mRNA, use, kit or composition according to any one of clauses 221 to 225, wherein at least half of the mRNA is incorporated into or associated with the LION, suitably at least 85%, especially at least 95%, such as all of it.
Clause 227. The method, emulsion, carrier-formulated mRNA, use, kit or composition according to any one of clauses 1 to 226, wherein the carrier-formulated mRNA and squalene emulsion adjuvant are administered as a co-formulation.
Clause 228. The method, emulsion, carrier-formulated mRNA, use, kit or composition according clause 227, wherein the volume of a single dose of carrier-formulated mRNA and squalene emulsion adjuvant is 0.05 ml to 1 ml, such as 0.1 to 0.6 ml.
Clause 229. The method, emulsion, carrier-formulated mRNA, use, kit or composition according clause 228, wherein the volume of a single dose of carrier-formulated mRNA and squalene emulsion adjuvant is 0.2 to 0.3 ml, such as 0.25 ml.
Clause 230. The method, emulsion, carrier-formulated mRNA, use, kit or composition according clause 228, wherein the volume of a single dose of carrier-formulated mRNA and squalene emulsion adjuvant is 0.4 to 0.6 ml, such as 0.5 ml.
Clause 231. The method, emulsion, carrier-formulated mRNA, use, kit or composition according to any one of clauses 227 to 230, for intramuscular administration.
Clause 232. The method, emulsion, carrier-formulated mRNA, use, kit or composition according to any one of clauses 227 to 230, wherein the co-formulation has a pH of 4 to 9, especially 5 to 8.5, in particular 5.5 to 8, such as 6.5 to 7.4.
Clause 233. The method, emulsion, carrier-formulated mRNA, use, kit or composition according to any one of clauses 227 to 232, wherein the co-formulation has an osmolality of 250 to 750 mOsm/kg, especially 250 to 550 mOsm/kg, in particular 270 to 500 mOsm/kg, such as 270 to 400 mOsm/kg.
Clause 234. The method, emulsion, carrier-formulated mRNA, use, kit or composition according to any one of clauses 227 to 233, wherein the concentration of squalene is 0.8 to 100 mg per ml, especially 1.2 to 48.4 mg per ml.
Clause 235. The method, emulsion, carrier-formulated mRNA, use, kit or composition according to any one of clauses 1 to 8 or 11 to 226, wherein the carrier-formulated mRNA and squalene emulsion adjuvant are administered as a separate formulations.
Clause 236. The method, emulsion, carrier-formulated mRNA, use, kit or composition according to clause 235, wherein the volume of a single dose of squalene emulsion adjuvant is 0.05 ml to 1 ml, such as 0.1 to 0.6 ml.
Clause 237. The method, emulsion, carrier-formulated mRNA, use, kit or composition according to clause 236, wherein the volume of a dose of the squalene emulsion adjuvant is 0.2 to 0.3 ml, such as 0.25 ml.
Clause 238. The method, emulsion, carrier-formulated mRNA, use, kit or composition according to clause 236, wherein the volume of a dose of the squalene emulsion adjuvant is 0.4 to 0.6 ml, such as 0.5 ml.
Clause 239. The method, emulsion, carrier-formulated mRNA, use, kit or composition according to any one of clauses 235 to 238, wherein the squalene emulsion adjuvant is for intramuscular administration.
Clause 240. The method, emulsion, carrier-formulated mRNA, use, kit or composition according to any one of clauses 235 to 239, wherein the squalene emulsion adjuvant has a pH of 4 to 9, especially 5 to 8.5, in particular 5.5 to 8, such as 6.5 to 7.4.
Clause 241. The method, emulsion, carrier-formulated mRNA, use, kit or composition according to any one of clauses 235 to 240, wherein the squalene emulsion adjuvant has an osmolality of 250 to 750 mOsm/kg, especially 250 to 550 mOsm/kg, in particular 270 to 500 mOsm/kg, such as 270 to 400 mOsm/kg.
Clause 242. The method, emulsion, carrier-formulated mRNA, use, kit or composition according to any one of clauses 235 to 241, wherein the squalene emulsion adjuvant comprises a buffer and/or tonicity modifying agents, such as modified phosphate buffered saline.
Clause 243. The method, emulsion, carrier-formulated mRNA, use, kit or composition according to any one of clauses 235 to 242, wherein the concentration of squalene is 0.8 to 100 mg per ml, especially 1.2 to 48.4 mg per ml.
Clause 244. The method, emulsion, carrier-formulated mRNA, use, kit or composition according to any one of clauses 235 to 243, wherein the volume of a dose of carrier-formulated mRNA is 0.05 ml to 1 ml, such as 0.1 to 0.6 ml.
Clause 245. The method, emulsion, carrier-formulated mRNA, use, kit or composition according to clause 244, wherein the volume of a dose of carrier-formulated mRNA is 0.2 to 0.3 ml, such as 0.25 ml.
Clause 246. The method, emulsion, carrier-formulated mRNA, use, kit or composition according to clause 244, wherein the volume of a dose of carrier-formulated mRNA is 0.4 to 0.6 ml, such as 0.5 ml.
Clause 247. The method, emulsion, carrier-formulated mRNA, use, kit or composition according to any one of clauses 235 to 246, wherein the carrier-formulated mRNA is for intramuscular administration.
Clause 248. The method, emulsion, carrier-formulated mRNA, use, kit or composition according to any one of clauses 235 to 247, wherein the carrier-formulated mRNA has a pH of 4 to 9, especially 5 to 8.5, in particular 5.5 to 8, such as 6.5 to 7.4.
Clause 249. The method, emulsion, carrier-formulated mRNA, use, kit or composition according to any one of clauses 235 to 248, wherein the carrier-formulated mRNA has an osmolality of 250 to 750 mOsm/kg, especially 250 to 550 mOsm/kg, in particular 270 to 500 mOsm/kg, such as 270 to 400 mOsm/kg.
Clause 250. The method, emulsion, carrier-formulated mRNA, use, kit or composition according to any one of clauses 235 to 249, wherein the carrier-formulated mRNA comprises a buffer and/or tonicity modifying agents, such as modified phosphate buffered saline.
Clause 251. The method, emulsion, carrier-formulated mRNA, use, kit or composition according to any one of clauses 235 to 250, wherein the carrier-formulated mRNA and squalene emulsion adjuvant are administered to a location draining to the same lymph node, such as to the same limb, in particular to the same muscle.
Clause 252. The method, emulsion, carrier-formulated mRNA, use, kit or composition according to any one of clauses 235 to 251, wherein the carrier-formulated mRNA and squalene emulsion adjuvant are administered to the same location.
Clause 253. The method, emulsion, carrier-formulated mRNA, use, kit or composition according to any one of clauses 235 to 252, wherein the carrier-formulated mRNA is administered before the squalene emulsion adjuvant.
Clause 254. The method, emulsion, carrier-formulated mRNA, use, kit or composition according to any one of clauses 235 to 252, wherein the squalene emulsion adjuvant is administered before the carrier-formulated mRNA.
Clause 255. The method, emulsion, carrier-formulated mRNA, use, kit or composition according to any one of clauses 235 to 254, wherein the squalene emulsion adjuvant and the carrier-formulated mRNA are administered within 12 hours, especially within 6 hours, such as within 2 hours of each other.
Clause 256. The method, emulsion, carrier-formulated mRNA, use, kit or composition according to any one of clauses 235 to 254, wherein the squalene emulsion adjuvant and the carrier-formulated mRNA are administered within 12 to 36 hours of each other.
Clause 257. The method, emulsion, carrier-formulated mRNA, use, kit or composition according to any one of clauses 235 to 254, wherein the squalene emulsion adjuvant and the carrier-formulated mRNA are administered within 36 to 84 hours of each other.
Clause 258. The method, emulsion, carrier-formulated mRNA, use, kit or composition according to any one of clauses 235 to 254, wherein the carrier-formulated mRNA and squalene emulsion adjuvant are administered concurrently.
Clause 259. The method, emulsion, carrier-formulated mRNA, use, kit or composition according to any one of clauses 1 to 258, wherein administration of the carrier-formulated mRNA and the squalene emulsion adjuvant induces an immune response that is equivalent to that induced by an at least 2-fold, such as at least 5-fold greater quantity of carrier-formulated mRNA without squalene emulsion adjuvant.
Clause 260. The method, emulsion, carrier-formulated mRNA, use, kit or composition according to any one of clauses 1 to 259, wherein administration of the carrier-formulated mRNA and the squalene emulsion adjuvant results in reduced reactogenicity compared to that induced by an at least 2-fold, such as at least 5-fold greater quantity of carrier-formulated mRNA without squalene emulsion adjuvant.
Clause 261. The method, emulsion, carrier-formulated mRNA, use, kit or composition according to any one of clauses 1 to 260, for administration to a subject which is not infected with SARS-CoV-2.
Clause 262. The method, emulsion, carrier-formulated mRNA, use, kit or composition according to any one of clauses 1 to 261, wherein administration is for administration to a subject which is not infected with a coronavirus.
Clause 263. The method, emulsion, carrier-formulated mRNA, use, kit or composition according to any one of clauses 1 to 262, for use in prophylaxis of an infectious disease, such as COVID-19.
Clause 264. The method, emulsion, carrier-formulated mRNA, use, kit or composition according to any one of clauses 1 to 263, wherein the elicited immune response reduces partially or completely the severity of one or more symptoms and/or time over which one or more symptoms are experienced by the subject.
Clause 265. The method, emulsion, carrier-formulated mRNA, use, kit or composition according to any one of clauses 1 to 264, wherein the elicited immune response reduces the likelihood of developing an established infection after challenge.
Clause 266. The method, emulsion, carrier-formulated mRNA, use, kit or composition according to any one of clauses 1 to 265, wherein the elicited immune response slows progression of illness (e.g. extends survival).
Clause 267. The method, emulsion, carrier-formulated mRNA, use, kit or composition according to any one of clauses 1 to 266, wherein the elicited immune response produces neutralising antibodies.
Clause 268. The method, emulsion, carrier-formulated mRNA, use, kit or composition according to any one of clauses 1 to 267, wherein the elicited immune response is an antigen specific T cell response.
Clause 269. The method, emulsion, carrier-formulated mRNA, use, kit or composition according to any one of clauses 1 to 268, wherein the squalene emulsion adjuvant does not comprise a GLA or SLA, such as does not comprise a TLR4 agonist, especially does not comprise additional immunostimulants other than tocopherol (if present).
Clause 270. The method, emulsion, carrier-formulated mRNA, use, kit or composition according to any one of clauses 1 to 269, wherein a formulation comprising the squalene emulsion adjuvant does not comprise a GLA or SLA, such as does not comprise a TLR4 agonist, especially does not comprise additional immunostimulants other than tocopherol (if present).
Clause 271. The method, emulsion, carrier-formulated mRNA, use, kit or composition according to any one of clauses 1 to 270, wherein the squalene emulsion adjuvant does not comprise poloxamer 188.
Clause 272. The method, emulsion, carrier-formulated mRNA, use, kit or composition according to any one of clauses 1 to 271, wherein the antigen is not a Zika virus pre-M-E antigen (prME), or an immunogenic fragment or variant thereof, especially the antigen is not a Zika virus antigen.
Clause 273. The method, emulsion, carrier-formulated mRNA, use, kit or composition according to any one of clauses 1 to 272, wherein the antigen is not a respiratory syncytial virus (RSV) F antigen, especially the antigen is not an RSV antigen.
Clause 274. The method, emulsion, carrier-formulated mRNA, use, kit or composition according to any one of clauses 1 to 273, wherein the antigen is not a human immunodeficiency virus (HIV) gp140 antigen, especially the antigen is not an HIV antigen.
Clause 275. The method, emulsion, carrier-formulated mRNA, use, kit or composition according to any one of clauses 1 to 274, wherein the antigen is not a Leishmania antigen.
Clause 276. The method, emulsion, carrier-formulated mRNA, use, kit or composition according to any one of clauses 1 to 275, wherein the antigen is not a glycoprotein.
Clause 277. The method, emulsion, carrier-formulated mRNA, use, kit or composition according to any one of clauses 1 to 276, wherein the carrier is not an LNP.
Clause 278. The method, emulsion, carrier-formulated mRNA, use, kit or composition according to any one of clauses 1 to 277, wherein the carrier is CNE and the CNE carrier does not comprise DOTAP.
Clause 279. The method, emulsion, carrier-formulated mRNA, use, kit or composition according to any one of clauses 1 to 277, wherein the carrier is not CNE.
Clause 280. The method, emulsion, carrier-formulated mRNA, use, kit or composition according to any one of clauses 1 to 279, wherein the carrier is not LION, especially the carrier does not comprise iron oxide particles, in particular the carrier does not comprise inorganic oxide particles.
Clause 281. The method, emulsion, carrier-formulated mRNA, use, kit or composition according to any one of clauses 1 to 280, wherein the squalene emulsion adjuvant does not comprise a cationic lipid.
Clause 282. The method, emulsion, carrier-formulated mRNA, use, kit or composition according to any one of clauses 1 to 281, wherein the squalene emulsion adjuvant does not encapsulate or complex at least 50%, especially at least 85%, particularly at least 90% such at least 95% of the mRNA.
Clause 283. The method, emulsion, carrier-formulated mRNA, use, kit or composition according to any one of clauses 1 to 282, wherein administration of the carrier-formulated mRNA in conjunction with the squalene emulsion adjuvant does not significantly alter (such as less than +/−25% change, especially less than +/−10% change and in particular less than +/−5% change) the level of antigen expression observed in the absence of squalene emulsion adjuvant.
Clause 284. The method, emulsion, carrier-formulated mRNA, use, kit or composition according to any one of clauses 1 to 283, wherein the squalene emulsion adjuvant is not the carrier.
Clause 285. The method, emulsion, carrier-formulated mRNA, use, kit or composition according to any one of clauses 1 to 284, wherein the antigen is a human cytomegalovirus (CMV) antigen.
Clause 286. The method, emulsion, carrier-formulated mRNA, use, kit or composition according to any one of clauses 1 to 284, wherein the antigen is a Zika virus antigen.
Clause 287. The method, emulsion, carrier-formulated mRNA, use, kit or composition according to any one of clauses 1 to 284, wherein the antigen is a human parainfluenza virus (PIV) antigen, such as a human PIV type 3 antigen.
Clause 288. The method, emulsion, carrier-formulated mRNA, use, kit or composition according to any one of clauses 1 to 284, wherein the antigen is a human metapneumovirus (hMPV) antigen.
Clause 289. The method, emulsion, carrier-formulated mRNA, use, kit or composition according to any one of clauses 1 to 284, wherein the antigen is a respiratory syncytial virus (RSV) antigen.
Clause 290. The method, emulsion, carrier-formulated mRNA, use, kit or composition according to any one of clauses 1 to 284, wherein the antigen is an influenza virus antigen, such as a hemagglutinin or a neuraminidase.
Clause 291. The method, emulsion, carrier-formulated mRNA, use, kit or composition according to any one of clauses 1 to 284, wherein the antigen is an Epstein-Barr virus (EBV) antigen.
Clause 292. The method, emulsion, carrier-formulated mRNA, use, kit or composition according to any one of clauses 1 to 284, wherein the antigen is an HSV antigen.
Clause 293. The method, emulsion, carrier-formulated mRNA, use, kit or composition according to clause 292, wherein the antigen is gE antigen, such as a sequence comprising, such as consisting of, a sequence having at least 80%, such as at least 90%, especially at least 95%, in particular at least 98% for example at least 99% or 100% identity to SEQ ID No: 9.
Clause 294. The method, emulsion, carrier-formulated mRNA, use, kit or composition according to clause 292, wherein the antigen is gl antigen, such as a sequence comprising, such as consisting of, a sequence having at least 80%, such as at least 90%, especially at least 95%, in particular at least 98% for example at least 99% or 100% identity to SEQ ID No: 10.
Clause 295. The method, emulsion, carrier-formulated mRNA, use, kit or composition according to any one of clauses 1 to 262 or 264 to 294, for use in treatment, such as the treatment of an infectious disease.

### EXAMPLES

### Example 1—Squalene Emulsion Manufacture

Oil phase composed of squalene and D/L-alpha tocopherol was formulated under a nitrogen atmosphere. Aqueous phase, composed of modified phosphate buffered saline and polysorbate 80, was prepared separately. Oil and aqueous phases were combined at a ratio of 1:9 (volume of oil phase to volume of aqueous phase) before homogenisation and microfluidisation (three passes through a microfluidiser at around 15000 psi). The resulting emulsion was sterile filtered through two trains of two 0.5/0.2 um filters in series (i.e. 0.5/0.2/0.5/0.2).

A final content of around 42.76 mg/ml squalene, 47.44 mg tocopherol and 19.44 mg/ml polysorbate 80 was targeted, i.e. double strength AS03_{A} based on a 500 ul dose volume.

Particle size and polydispersity was determined by DLS to be within the range 140 to 180 nm and less than 0.2 respectively. Squalene and tocopherol content was confirmed by HPLC and polysorbate 80 content by spectrophotometry to be within specification.

### Example 2—Immunogenicity Evaluation of SAM Administered with Squalene Emulsion Adjuvant in Mice

### Materials and Methods

### Mouse Immunisation, Vector Production and Vaccination Scheme

Female CB6F1 inbred mice aged 6-8 weeks old were randomly assigned to study groups (n=8 for groups 1-14 (gr1-14) and n=4 for group 15 (gr15)) and kept under pathogen-free conditions. Mice (gr1-14) were intramuscularly (i.m.) immunized, in the gastrocnemius muscle, at days 0 & 28 with 0.1 ug or 0.01 ug of LNP-formulated SAM (25 uL volume) comprising a heterodimer of the HSV-2 gE and gl proteins (LNP/SAM-gE_P317R/gl) and were injected, in the same muscle, with different doses of squalene emulsion adjuvant (AS03) prepared as detailed above under Example 1 (5 uL of emulsion, made up to 25 uL with PBS, or 25 uL of emulsion) either at the same time (day+0) or 1 or 3 days after each of the two SAM immunizations. As positive control for vaccine response, mice immunized with both doses of LNP/SAM-gE_P317R/gl vaccine were i.m. injected, at the same time as the immunization, with a saline solution (NaCl 150 mM, 25 uL). Finally, as a negative control, mice were i.m injected with only 50 uL of a saline solution (NaCl 150 mM), following the same schedule of immunization (see Table 1 for details of each group).

TABLE 1
Summary of the study design and formulations tested

| | | | Vaccine | Adjuvant | |
|---|---|---|---|---|---|
| | | | immunization | dose and | Volume and |
| | Number of | | schedule | immunization | route of |
| Group | animals | Vaccine name & dose | (Days) | schedule | injection |
| 1 | 8 | LNP/SAM-gE_P317R/gl | Days 0 & 28 | 25 uL AS03 | i.m. |
| | | (0.1 ug) | | Day + 0 | 25 uL each |
| 2 | 8 | LNP/SAM-gE_P317R/gl | Days 0 & 28 | 25 uL AS03 | i.m. |
| | | (0.1 ug) | | Day + 1 | 25 uL each |
| 3 | 8 | LNP/SAM-gE_P317R/gl | Days 0 & 28 | 25 uL AS03 | i.m. |
| | | (0.1 ug) | | Day + 3 | 25 uL each |
| 4 | 8 | LNP/SAM-gE_P317R/gl | Days 0 & 28 | 5 uL AS03 | i.m. |
| | | (0.1 ug) | | Day + 0 | 25 uL each |
| 5 | 8 | LNP/SAM-gE_P317R/gl | Days 0 & 28 | 5 uL AS03 | i.m. |
| | | (0.1 ug) | | Day + 1 | 25 uL each |
| 6 | 8 | LNP/SAM-gE_P317R/gl | Days 0 & 28 | 5 uL AS03 | i.m. |
| | | (0.1 ug) | | Day + 3 | 25 uL each |
| 7 | 8 | LNP/SAM-gE_P317R/gl | Days 0 & 28 | 25 uL AS03 | i.m. |
| | | (0.01 ug) | | Day + 0 | 25 uL each |
| 8 | 8 | LNP/SAM-gE_P317R/gl | Days 0 & 28 | 25 uL AS03 | i.m. |
| | | (0.01 ug) | | Day + 1 | 25 uL each |
| 9 | 8 | LNP/SAM-gE_P317R/gl | Days 0 & 28 | 25 uL AS03 | i.m. |
| | | (0.01 ug) | | Day + 3 | 25 uL each |
| 10 | 8 | LNP/SAM-gE_P317R/gl | Days 0 & 28 | 5 uL AS03 | i.m. |
| | | (0.01 ug) | | Day + 0 | 25 uL each |
| 11 | 8 | LNP/SAM-gE_P317R/gl | Days 0 & 28 | 5 uL AS03 | i.m. |
| | | (0.01 ug) | | Day + 1 | 25 uL each |
| 12 | 8 | LNP/SAM-gE_P317R/gl | Days 0 & 28 | 5 uL AS03 | i.m. |
| | | (0.01 ug) | | Day + 3 | 25 uL each |
| 13 | 8 | LNP/SAM-gE_P317R/gl | Days 0 & 28 | (NaCl 150 mM) | i.m. |
| | | (0.1 ug) | | Day + 0 | 25 uL each |
| 14 | 8 | LNP/SAM-gE_P317R/gl | Days 0 & 28 | (NaCl 150 mM) | i.m. |
| | | (0.01 ug) | | Day + 0 | 25 uL each |
| 15 | 4 | NaCl 150 mM | Days 0 & 28 | (NaCl 150 mM) | i.m. |
| | | | | Day + 0 | 25 uL each |

Serum samples were collected at days 25 & 50 post-prime immunization (25PI, 22P11) to measure anti-HSV-2 gE- and gl-specific IgG antibody responses and investigate the function of vaccine-specific antibodies. Spleens were also collected 22 days post second immunization (22P11) to evaluate vaccine-specific CD4+/CD8+ T cell responses. The study was split across two independent sites—Experiment A and Experiment B.

The SAM pTC83R_P989 plasmid (pRIT17148, HSV-2_gE_P317R_IRES_gl SAM) was designed as a bicistronic vector where i) gE expression is driven by a single promoter (subgenomic promoter) and ii) g expression was driven by an Internal Ribosome Entry Site from enterovirus 71 (IRES EV71). Moreover, in order to knock down Fc binding activity, mutation P317R was introduced in the gE sequence. HSV-2 gE/g sequences were codon optimized for expression in humans. For the gE sequence, nucleotides (nt) from 1 to 1257 were included in the construction and for g sequence, nt from 1 to 768. The nucleotide sequence of the HSV-2_gEpP317R_IRES_gl insert is provided in SEQ ID NO: 5. The insert comprises ApaI, NotI, Tth111I and stBI restriction sites and a double stop codon.

The nucleotide sequence of the parent SAM pTC83R_P989 plasmid (comprising SAM backbone sequences which are not incorporated into the SAM gE_P317R_IRES_gl transcript) is provided in SEQ ID NO: 6 and comprises the functional elements shown in Table 2. The polynucleotide sequence of SAM gE_P317R_IRES_gl is provided in SEQ ID NO: 7. The SAM pTC83R_P989 transcript theoretical RNA sequence is provided in SEQ ID NO: 8. This sequence is 5-capped with 7-methylguanosine (Cap 0). The resultant polynucleotide is referred to herein as SAM-gE_P317R/gl. When administered to mice in LNP, this is referred to herein as LNP/SAM-gE_P317R/gl. This polynucleotide encodes the gE P317R and gl amino acid sequences which are provided in SEQ ID NO: 9 and 10, respectively.

TABLE 2
SAM pTC83R_P989 plasmid functional elements

| Position | Name |
|---|---|
| 10482..12775 | E. coli vector backbone |
| 12758..12774 | T7 promoter |
| 12775 | Transcription start site |
| 12775..10521 | SAM HSV-2 gE/gl primary transcript |
| 1..44 | 5′UTR (5′-untranslated region) |
| 45..7526 | Non-structural polyprotein nsP1234 |
| 7513..7536 | Subgenomic promoter |
| 7501..7506 | Apal upstream cloning site |
| 7541..7549 | Tth111I site |
| 7537..7561 | UTR upstream of the target gene's coding sequence |
| 7562..9136 | Coding sequence of the target gene |
| | (gE P317R_IRES_gl) |
| 8922..8927 | BstBI site |
| 10340..10364 | Synthetic vector polylinker with NotI downstream |
| | cloning site |
| 10365..10481 | 3′UTR (3′-untranslated region) |
| 10482..10522 | Synthetic 3′-polyA sequence |
| Complement: | BspQI site |
| 10523-10530 | |

SAM-LNP was prepared by rapidly mixing ethanolic solutions of lipids with aqueous buffers that contain SAM. The rapid mixing resulted in a supersaturation of hydrophobic components that have ionically paired with SAM. The SAM/lipid complexes condense and precipitate through nucleation mediated precipitation, yielding small and narrowly disperse nanoparticles. Following this mixing step, the lipid nanoparticles matured to entrap the RNA and then were transferred to a final Tris/sucrose storage buffer through a buffer exchange step. The LNP solutions were then characterized for size, lipid content, RNA entrapment, final SAM concentration, and in vitro potency. The LNP solutions comprised 20 mM Tris, 5 mM NaCl, 7.5% sucrose, and had a pH of 8.

### Detection of Total Anti-HSV-2 gE & Gl IgG Antibodies by ELISA

Quantification of the total HSV-2 gE or gl-specific IgG antibodies was performed using indirect ELISA. Recombinant HSV-2 gE (˜51 kDa) or HSV-2 gl proteins (˜46 kDa) were used as coating antigens.

Polystyrene 96-well ELISA plates were coated with 100 uL/well of antigen diluted at a concentration of 2 ug/mL (HSV-2 gE) and 1 ug/mL (HSV-2 gl) in carbonate/bicarbonate 50 mM pH 9.5 buffer and incubated overnight at 4° C. After incubation, the coating solution was removed and the plates were blocked with 200 uL/well of skimmed milk media additive 10% diluted in PBS (blocking buffer) for 1 h at 37° C. The blocking solution was removed and the two-fold (sera 25PI) or three-fold (sera 22PII) sera dilutions (in PBS+0.1% Tween20+1% BSA buffer) were added to the coated plates and incubated for 1 h at 37° C. The plates were washed four times with PBS 0.1% Tween20 (washing buffer) and peroxidase conjugated goat anti-mouse IgG (H+L) was used as secondary antibody. One hundred microliters per well of the secondary antibody diluted at a concentration of 1:500 in PBS+0.1% Tween20+1% BSA buffer was added to each well and the plates were incubated for 45 min at 37° C.

The plates were then washed four times with washing buffer and 2 times with deionised water and incubated for 15 min at RT (room temperature) with 100 uL/well of a solution of 75% single-component TMB peroxidase ELISA substrate diluted in sodium citrate 0.1M pH5.5 buffer. Enzymatic color development was stopped with 100 uL of 0.4N sulfuric acid 1M (H₂SO₄) per well and the plates were read at an absorbance of 450/620 nm using an ELISA reader.

Optical densities (OD) were captured and analysed. A standard curve was generated by applying a 4-parameter logistic regression fit to a reference standard. Antibody titer in the samples was calculated by interpolation of the standard curve. The antibody titer of the samples was obtained by averaging the values from dilutions that fell within the 20-80% dynamic range of the standard curve. Antibody titers were normalized at the same starting dilution.

### Evaluation of Anti-HSV-2 gE and gl CD4+/CD8+ T Cell Responses by Intracellular Cytokine Staining (ICS)

The frequencies of vaccine-specific CD4+ and CD8+ T-cells producing IL-2 and/or IFN-γ and/or TNF-α were evaluated in splenocytes collected 22 days post-second immunization after ex-vivo stimulation with HSV-2 gE or gl peptide pools.

### Isolation of Splenocytes

Spleens were collected from individual mice 22 days after second immunization and placed in RPMI 1640 medium supplemented with RPMI additives (glutamine, penicillin/streptomycin, sodium pyruvate, non-essential amino-acids & 2-mercaptoethanol) (=RPMI/additives). Cell suspensions were prepared from each spleen using a tissue grinder. The splenic cell suspensions were filtered (cell stainer 100 um) and then the filter was rinsed with 35 mL of cold PBS-EDTA. After centrifugation (335 g, 10 min at 4° C.), cells were resuspended in 5 mL of cold PBS-EDTA. A second washing step was performed, as previously described, and the cells were finally resuspended in 2 mL of RPMI/additives supplemented with 5% FCS. Cell suspensions were then diluted 20× (10 uL) in PBS buffer (190 uL) for cell counting. After counting, cells were centrifuged (335 g, 10 min at RT) and resuspended at 10⁷ cells/mL in RPMI/additives supplemented with 5% FCS.

### Cell Preparation

Fresh splenocytes were seeded in round bottom 96-well plates at 10⁶ cells/well (100 uL). The cells were then stimulated for 6 hours (37° C., 5% CO₂) with anti-CD28 and anti-CD49d antibodies at 1 ug/mL per well, containing 100 uL of either:
15 mer overlapping peptide pool covering the sequences of gE protein from HSV-2 (1 ug/mL per peptide per well).
15 mer overlapping peptide pool covering the sequences of gl protein from HSV-2 (1 ug/mL per peptide per well).
15 mer overlapping peptide pool covering the sequences of human p-actin protein (1 ug/mL per peptide per well) (irrelevant stimulation).
RPMI/additives medium (as negative control of the assay).
PMA—ionomycin solution at working concentrations of 0.25 ug/mL and 2.5 ug/mL respectively (as positive control of the assay).

After 2 hours of ex vivo stimulation, brefeldin A protein transport inhibitor diluted 1/200 in RPMI/additives supplemented with 5% FCS was added for 4 additional hours to inhibit cytokine secretion. Plates were then transferred at 4° C. for overnight incubation.

### Intracellular Cytokine Staining

After overnight incubation at 4° C., cells were transferred to V-bottom 96-well plates, centrifuged (189 g, 5 min at 4° C.) and washed with 250 uL of cold PBS+1% FCS (flow buffer). After a second centrifugation (189 g, 5 min at 4° C.), cells were resuspended to block unspecific antibody binding (10 min at 4° C.) in 50 uL of flow buffer containing anti-CD16/32 antibodies diluted 1/50. Then, 50 uL flow buffer containing mouse anti-CD4-V450 antibodies, anti-CD8-PerCp-Cy5.5 antibodies and fixable yellow dead cell stain was added for 30 min in darkness at 4° C. After incubation, 100 uL of flow buffer was added into each well and cells were then centrifuged (189 g for 5 min at 4° C.). A second washing step was performed with 200 uL of flow buffer and after centrifugation, cells were fixed and permeabilized by adding 200 uL of fixation solution for 20 min at 4° C. in darkness. After plate centrifugation (500 g for 5 min at 4° C.), cells were washed with 200 uL of cell wash buffer, centrifuged (500 g for 5 min 4° C.) and resuspended in 50 uL of cell wash buffer containing mouse anti-IL2-FITC, anti-IFN-γ-APC and anti-TNF-α-PE antibodies, for 1 hour at 4° C. in darkness. After incubation, 100 uL of flow buffer was added into each well and cells were then finally washed with 200 uL of cell wash buffer (centrifugation 500 g for 5 min at 4° C.) and resuspended in 220 uL PBS.

### Cell Acquisition and Analysis

Stained cells were acquired by flow cytometry and analyzed. Live cells were identified with staining and then lymphocytes were isolated based on forward/side scatter lights (FSC/SSC) gating. The acquisition was performed on ˜20,000 CD4+/CD8+ T-cell events. The percentages of IFN-γ^{+/-} IL-2^{+/-} and TNF-α^{+/-} producing cells were calculated on CD4+ and CD8+ T cell populations. For each sample, unspecific signal detected after medium stimulation was removed from the specific signal detected after peptide pool stimulation.

Competitive ELISA to Evaluate the Ability of Vaccine-Specific Antibodies to Decrease Human IgG Fc Binding by HSV-2 gE/Gl Protein

The ability of polyclonal sera collected in different groups of mice to decrease in-vitro hlgG Fc binding by recombinant HSV-2 gE/gl protein was investigated by competitive ELISA. Recombinant HSV-2 gE/gl protein was used as coating antigen.

Polystyrene 96-well ELISA plates were coated with 50 uL/well of HSV-2 gE/gl protein diluted at a concentration of 4 ug/mL in free calcium/magnesium PBS buffer and incubated overnight at 4° C. After incubation, the coating solution was removed and the plates were blocked with 100 uL/well of PBS supplemented with 0.1% Tween-20+1% BSA for 1 h at 37° C.

Sixty microliters (60 uL) of two serial dilution of individual mouse serum (starting dilution 1/10 in blocking buffer) was prepared into 96-well clear V-bottom polypropylene microplate and mixed with 60 uL/well of biotinylated-hlgG antibodies pre-diluted at 0.7 ug/mL in blocking buffer. The blocking buffer from HSV-2 gE/gl coated plates was removed and 100 uL of the mixture was then transferred in the corresponding wells for an incubation period of 24 h at 37° C. Positive control serum of the assay was a pool of serum samples from mice immunized with HSV-2 gE/gl antigen in previous experiments. Negative control serum for the assay was a pool of irrelevant HPV serum samples diluted 1/1000 and mixed with hlgG.

After incubation, the plates were washed four times with PBS 0.1% Tween20 (washing buffer) and 50 uL/well of streptavidin-horseradish peroxidase diluted 2000× was added on the well and plates were incubated for 30 min at 37° C. After incubation, plates were washed four times with washing buffer and 50 uL/well of a solution containing 75% single-component TMB peroxidase ELISA substrate diluted in sodium citrate 0.1M pH5.5 buffer were added for 10 min at room temperature. Enzymatic color development was stopped with 50 uL/well of 0.4N sulfuric acid 1M (H₂SO₄) and the plates were read at an absorbance of 450/620 nm using an ELISA reader. Optical densities (OD) were captured and fitted to a curve. Titers were expressed as the effective dilution at which 50% (i.e. ED50) of the signal was achieved by sample dilution. For each plate and using a reference sample (i.e. irrelevant serum), the reference ED50 value was estimated using the following formula:

ED50=OD_{0%}+0.5*(OD_{100%}−OD_{0%})

where OD_{100%} is the highest OD obtained with similar samples and OD_{0%} is the lowest achievable signal. For each plate, the former was obtained by averaging (mean) 6 replicates while the latter was set at zero. The samples' ED50 titers were computed by way of linear interpolation between the left and right measurements closest to the ED50 estimate within the plate. The approximation was obtained, on the untransformed OD and the logarithm base 10 transformed dilutions.

Samples were not assigned a titer in the following cases:
no measurement was available above or below the ED50,
curve crossed at least twice the ED50 and
one of the dilution step (left or right) closest to the ED50 was missing.

### Results

### Evaluation of Vaccine-Specific Antibody Responses

The anti-HSV-2 gE & gl-specific IgG antibody responses were investigated by ELISA at day 25 (25PI) and day 50 (22PII) and expressed in ELISA titers. The anti-HSV-2 gl IgG Ab response was not evaluated in serum samples from some mice in Group 1 (2 animals), Group 4 (2 animals), Group 5 (1 animal) and Group 8 (1 animal) at 25PI due to a lack of serum available.

Anti-HSV-2 gE-Specific IgG Antibodies

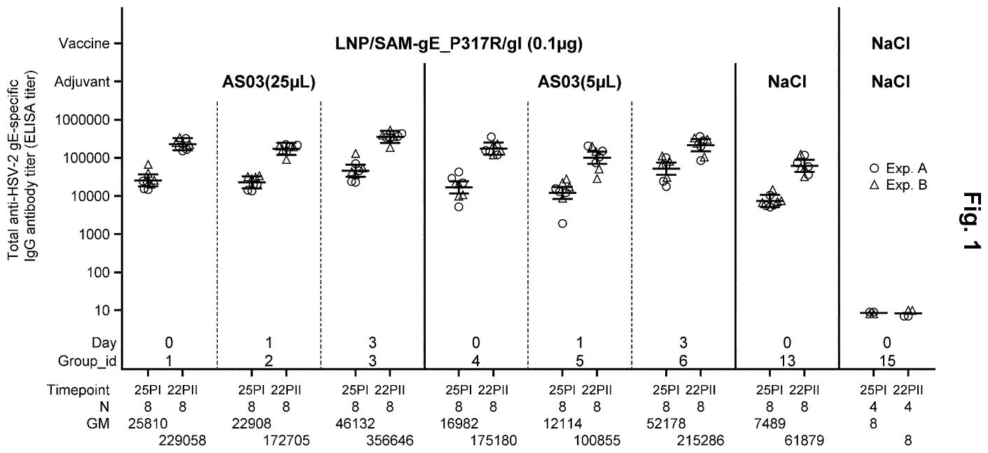

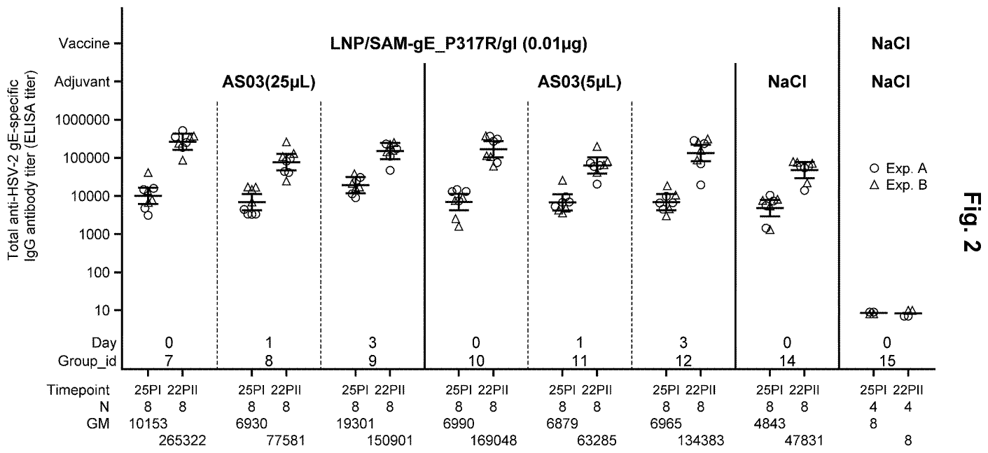

In all circumstances tested, AS03 adjuvanted LNP/SAM-gE_P317/gl resulted in increased GM titers of anti-HSV-2 gE-specific IgG antibodies, relative to unadjuvanted control (FIG. 1 and FIG. 2).

Anti-HSV-2 gl-Specific IgG Antibodies

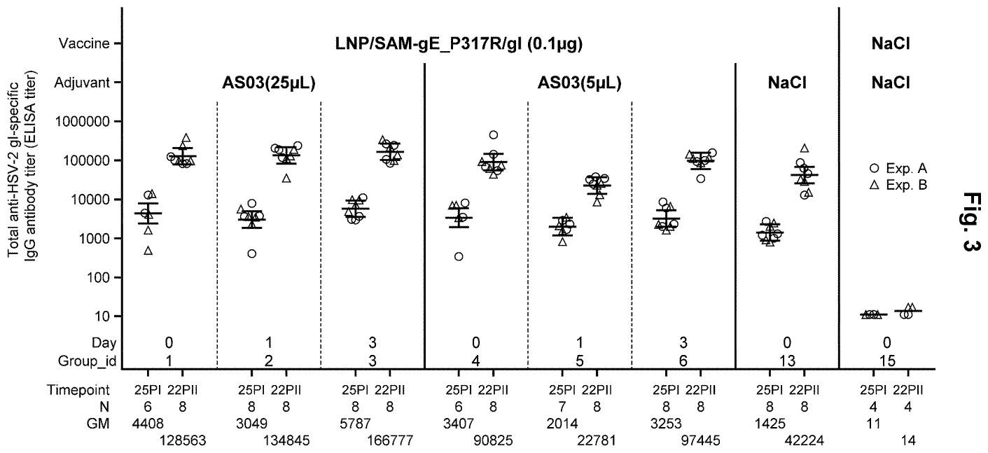

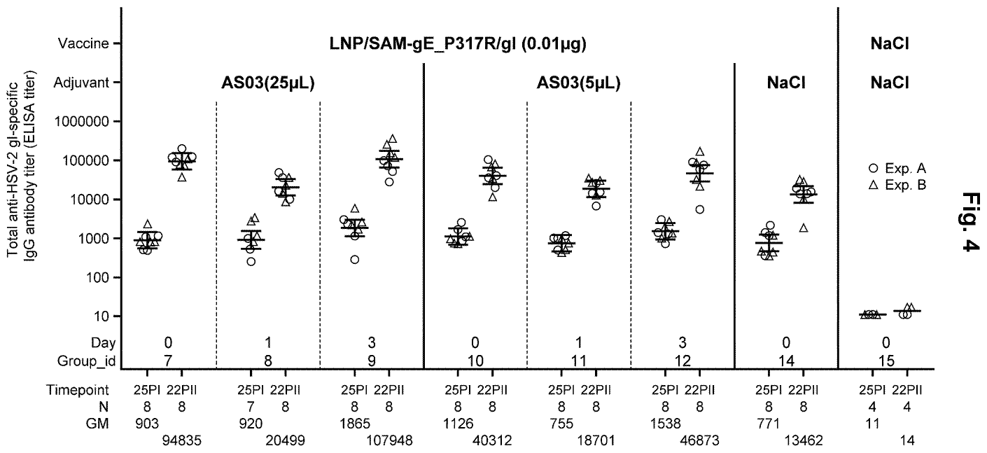

AS03 adjuvanted 0.1 ug LNP/SAM-gE_P317/gl resulted in increased GM titers of anti-HSV-2 gl-specific IgG antibodies in 5 of 6 groups at 22PII, relative to unadjuvanted control (FIG. 3). Adjuvanted 0.01 ug LNP/SAM-gE_P317/gl resulted in increased GM titers of anti-HSV-2 gl-specific IgG antibodies in all circumstances tested at 22PII, relative to unadjuvanted control (FIG. 4).

### Evaluation of Vaccine-Specific Antibody Function 22 Days Post-Second Vaccination

Vaccine-specific antibody functions were investigated in the sera collected at 22 days post-second immunization by assaying the ability of polyclonal antibodies to decrease binding of human IgG (hlgG) Fc by HSV-2 gE/gl protein.

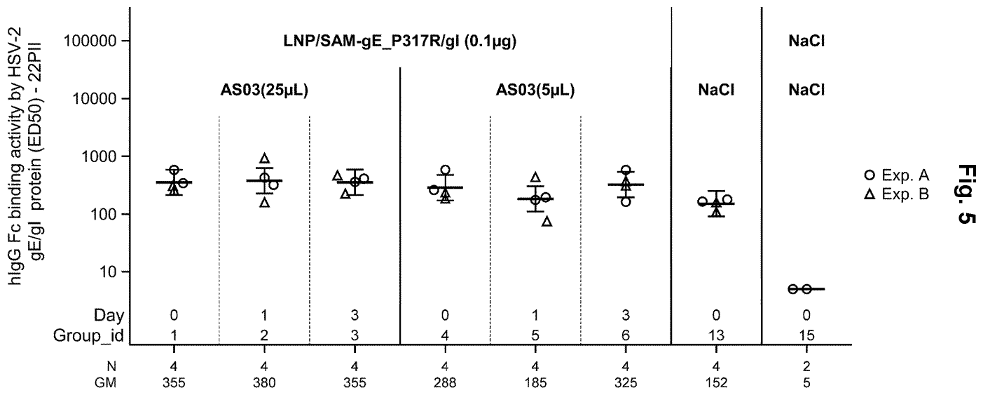

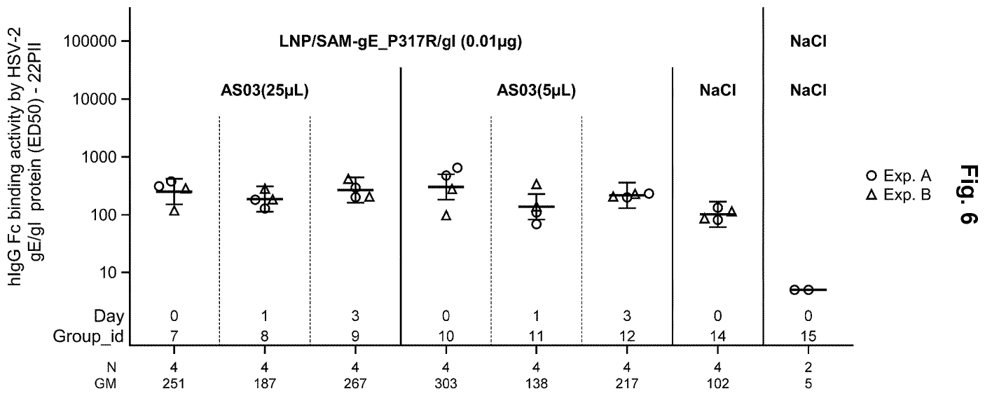

In all circumstances tested, AS03 adjuvanted LNP/SAM-gE_P317/gl resulted in increased GM ED50 values relative to unadjuvanted control (FIG. 5 and FIG. 6).

### Evaluation of Vaccine-Specific T Cell Responses 22 Days Post-Second Vaccination

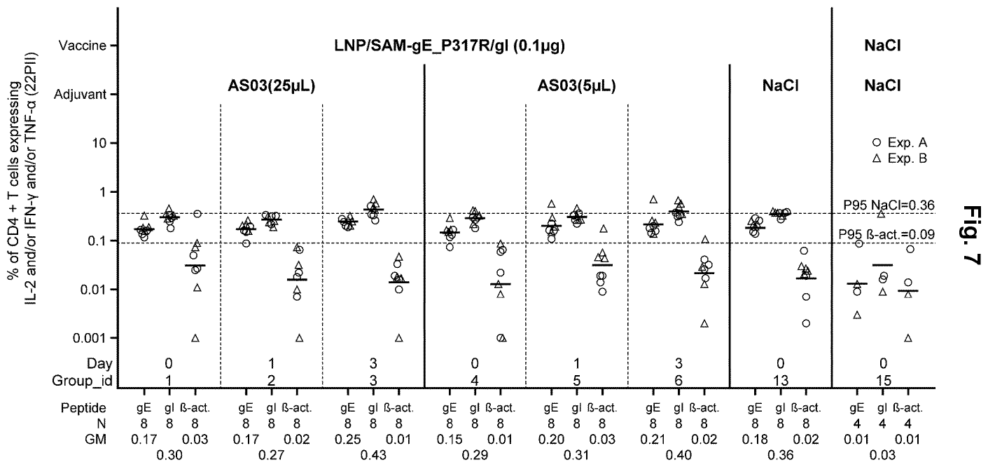

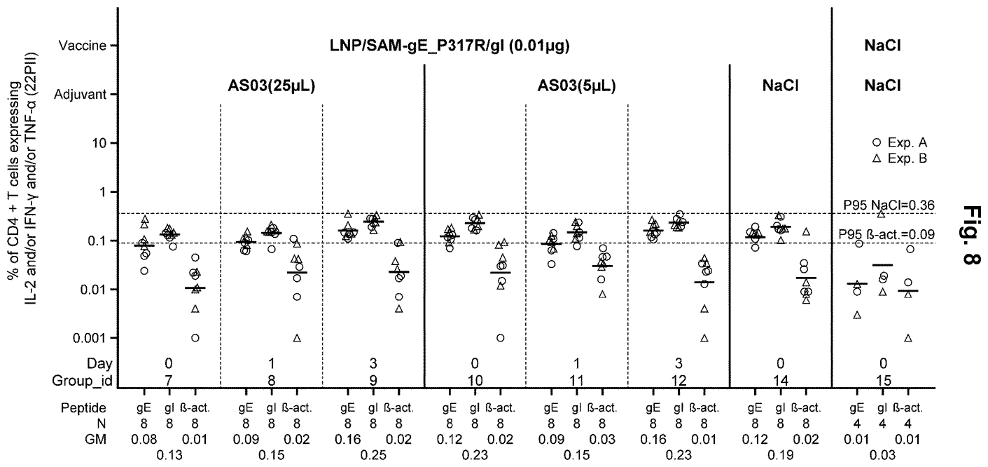

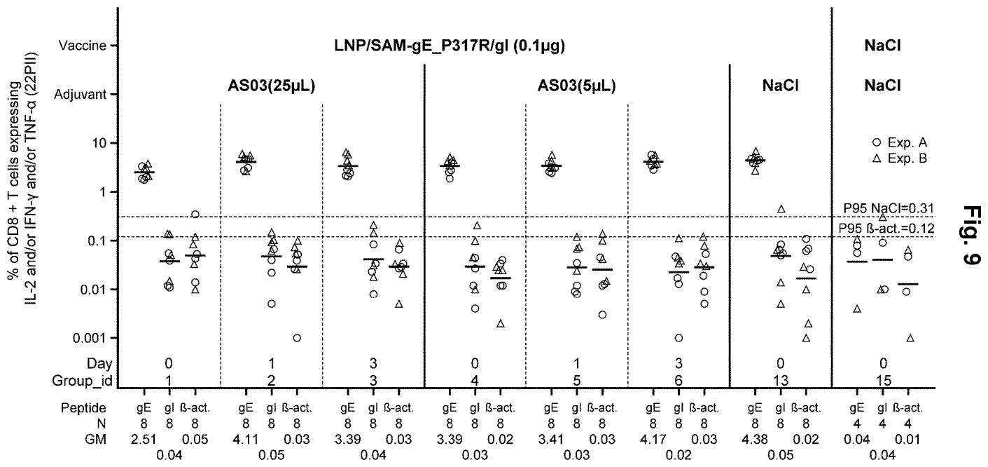

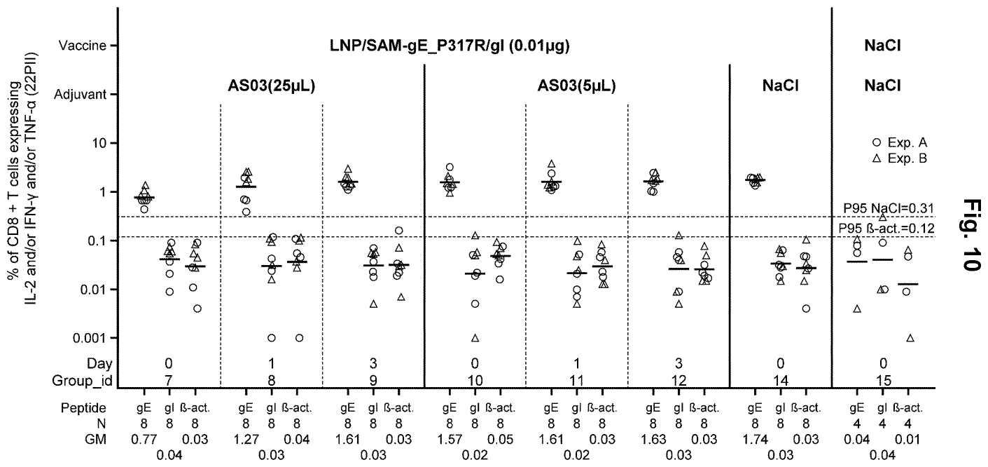

Twenty-two days after the second immunization (day 50), anti-HSV-2 gE & gl-specific CD4+/CD8+ T cell responses were measured in spleens from the immunized mice. Results are provided in FIGS. 7 to 10.

### Example 3—Immunogenicity Evaluation of SAM Administered with a Different Squalene Emulsion Adjuvant in Mice

### Materials and Methods

### Mouse Immunisation, Vector Production and Vaccination Scheme

Female CB6F1 inbred mice aged 6-8 weeks old were randomly assigned to the study groups (n=8 for groups 1-14 (gr1-14) and n=4 for group 15 (gr15)) and kept under specified pathogen-free conditions. Mice (gr1-14) were intramuscularly (i.m.) immunized, in the gastrocnemius muscle, at days 0 & 28 with 0.1 ug or 0.01 ug of LNP-formulated SAM-HSV-2 gE_P317R/gl (LNP/SAM-gE_P317R/gl) (25 uL volume) and were injected, in the same muscle, with different doses of AddaVax™ adjuvant (5 uL or 22.8 uL AddaVax™ diluted in PBS for a final injection volume of 25 uL) either at the same time or 1 or 3 days after each of the two SAM immunizations. As a positive control for vaccine response, mice immunized with both doses of LNP/SAM-gE_P317R/gl vaccine were i.m. injected, at the same time as the immunization, with a saline solution (NaCl 150 mM). Finally, as negative control, mice were only i.m injected with a saline solution (NaCl 150 mM), following the same schedule of immunization (see Table 3 for details of each group).

TABLE 3
Summary of the study design and formulations tested

| | | | Vaccine | | |
|---|---|---|---|---|---|
| | | | immunization | Adjuvant dose | Volume and |
| | Number of | | schedule | and immunization | route of |
| Group | animals | Vaccine name & dose | (Days) | schedule | injection |
| 1 | 8 | LNP/SAM-gE_P317R/gl | Days 0 & 28 | AddaVax ™ | i.m. |
| | | (0.1 ug) | | (22.8 uL in 2.2 uL | 25 uL each |
| | | | | PBS 10x cc pH 6.8) | |
| | | | | Day + 0 | |
| 2 | 8 | LNP/SAM-gE_P317R/gl | Days 0 & 28 | AddaVax ™ | i.m. |
| | | (0.1 ug) | | (22.8 uL in 2.2 uL | 25 uL each |
| | | | | PBS 10x cc pH 6.8) | |
| | | | | Day + 1 | |
| 3 | 8 | LNP/SAM-gE_P317R/gl | Days 0 & 28 | AddaVax ™ | i.m. |
| | | (0.1 ug) | | (22.8 uL in 2.2 uL | 25 uL each |
| | | | | PBS 10x cc pH 6.8) | |
| | | | | Day + 3 | |
| 4 | 8 | LNP/SAM-gE_P317R/gl | Days 0 & 28 | AddaVax ™ | i.m. |
| | | (0.1 ug) | | (5 uL in 20 uL PBS | 25 uL each |
| | | | | 10x cc pH 6.8) | |
| | | | | Day + 0 | |
| 5 | 8 | LNP/SAM-gE_P317R/gl | Days 0 & 28 | AddaVax ™ | i.m. |
| | | (0.1 ug) | | (5 uL in 20 uL PBS | 25 uL each |
| | | | | 10x cc pH 6.8) | |
| | | | | Day + 1 | |
| 6 | 8 | LNP/SAM-gE_P317R/gl | Days 0 & 28 | AddaVax ™ | i.m. |
| | | (0.1 ug) | | (5 uL in 20 uL PBS | 25 uL each |
| | | | | 10x cc pH 6.8) | |
| | | | | Day + 3 | |
| 7 | 8 | LNP/SAM-gE_P317R/gl | Days 0 & 28 | AddaVax ™ | i.m. |
| | | (0.01 ug) | | (22.8 uL in 2.2 uL | 25 uL each |
| | | | | PBS 10x cc pH 6.8) | |
| | | | | Day + 0 | |
| 8 | 8 | LNP/SAM-gE_P317R/gl | Days 0 & 28 | AddaVax ™ | i.m. |
| | | (0.01 ug) | | (22.8 uL in 2.2 uL | 25 uL each |
| | | | | PBS 10x cc pH 6.8) | |
| | | | | Day + 1 | |
| 9 | 8 | LNP/SAM-gE_P317R/gl | Days 0 & 28 | AddaVax ™ | i.m. |
| | | (0.01 ug) | | (22.8 uL in 2.2 uL | 25 uL each |
| | | | | PBS 10x cc pH 6.8) | |
| | | | | Day + 3 | |
| 10 | 8 | LNP/SAM-gE_P317R/gl | Days 0 & 28 | AddaVax ™ | i.m. |
| | | (0.01 ug) | | (5 uL in 20 uL PBS | 25 uL each |
| | | | | 10x cc pH 6.8) | |
| | | | | Day + 0 | |
| 11 | 8 | LNP/SAM-gE_P317R/gl | Days 0 & 28 | AddaVax ™ | i.m. |
| | | (0.01 ug) | | (5 uL in 20 uL PBS | 25 uL each |
| | | | | 10x cc pH 6.8) | |
| | | | | Day + 1 | |
| 12 | 8 | LNP/SAM-gE_P317R/gl | Days 0 & 28 | AddaVax ™ | i.m. |
| | | (0.01 ug) | | (5 uL in 20 uL PBS | 25 uL each |
| | | | | 10x cc pH 6.8) | |
| | | | | Day + 3 | |
| 13 | 8 | LNP/SAM-gE_P317R/gl | Days 0 & 28 | NaCl 150 mM | i.m. |
| | | (0.1 ug) | | Day + 0 | 25 uL each |
| 14 | 8 | LNP/SAM-gE_P317R/gl | Days 0 & 28 | NaCl 150 mM | i.m. |
| | | (0.01 ug) | | Day + 0 | 25 uL each |
| 15 | 4 | NaCl150 mM | Days 0 & 28 | NaCl 150 mM | i.m. |
| | | | | Day + 0 | 25 uL each |

Serum samples were collected at days 25 & 50 post prime immunization (25PI, 22PII) to measure anti-HSV-2 gE- and gl-specific IgG antibody responses and investigate the function of vaccine-specific antibodies. Spleens were also collected 22 days post second immunization (22PII) to evaluate vaccine-specific CD4+/CD8+ T cell responses. The study was split across two independent sites—Experiment A and Experiment B. LNP-formulated SAM-HSV-2 gE_P317R/gl (LNP/SAM-gE_P317R/gl) was identical to that used above in Example 2.

AddaVax™ is a squalene-based oil-in-water nano-emulsion based on the formulation of MF59®. AddaVax™ is available from Invivogen and is based on nano-emulsification of 2 components:
Sorbitan trioleate (0.5% w/v) in squalene oil (5% v/v)
Tween 80 (0.5% w/v) in sodium citrate buffer (10 mM, pH 6.5)
   The nano-emulsion is produced using a microfluidizer and filtered through a 0.22 um filter to remove large droplets and sterilize the final product. The particle size is approximately 160 nm. AddaVax™ is intended for dilution 1:1 by volume with an antigen composition.

Assays were carried out analogously to those detailed above in Example 2.

### Results

### Evaluation of Vaccine-Specific Antibody Responses

The anti-HSV-2 gE & gl-specific IgG antibody responses were investigated by ELISA at day 25 (25PI) and day 50 (22PII), and expressed in ELISA titers. The anti-HSV-2 gE IgG Ab response was not evaluated on serum samples from some mice in Group 5 (1 animal) and Group 13 (1 animal) (Experiment A) due to a lack of serum available. Also, the HSV-2 gl IgG Ab response was not evaluated in serum samples from some mice Group 1 (1 animal), Group 3 (1 animal), Group 5 (2 animals), Group 6 (1 animal), Group 8 (1 animal), Group 10 (2 animals), Group 12 (1 animal), Group 13 (2 animals) and Group 14 (1 animal) (Experiment A) and Group 7 (2 animals) and Group 9 (1 animal) (Experiment B) at 25PI immunization due to a lack of serum available.

Anti-HSV-2 gE-Specific IgG Antibodies

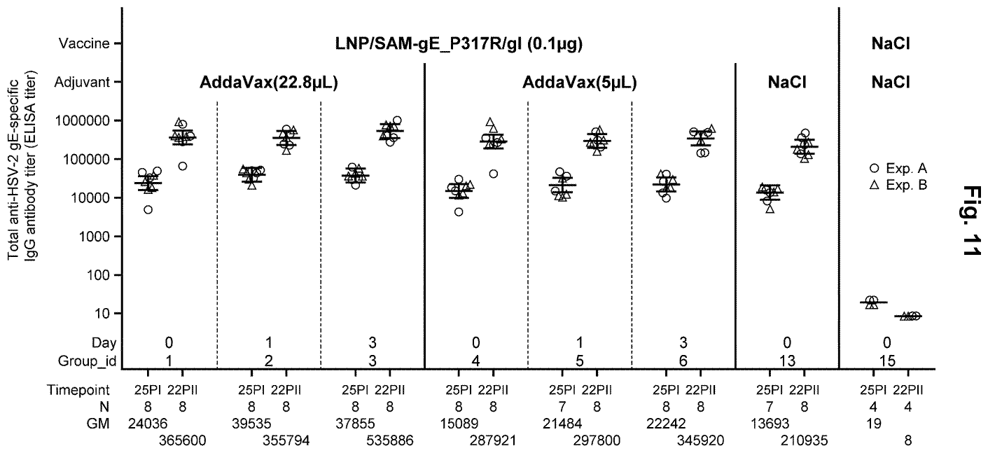

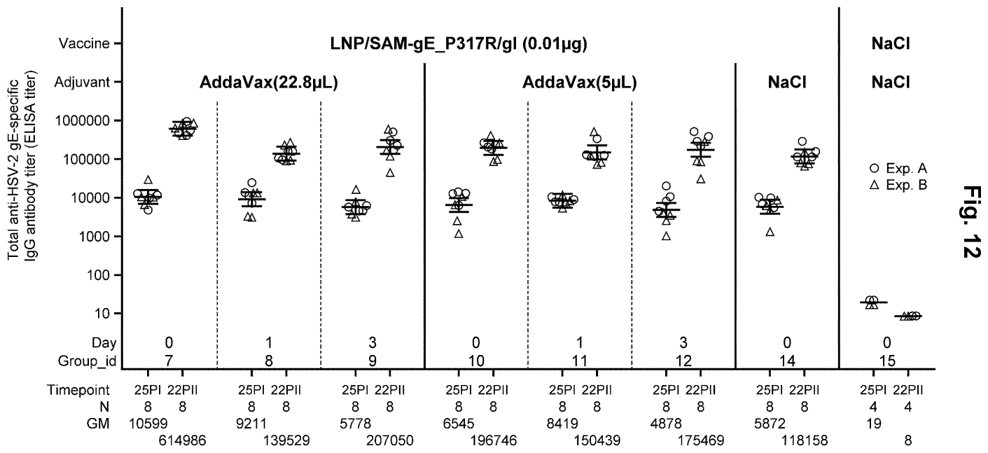

Administration of AddaVax™ adjuvanted LNP/SAM-gE_P317/gl resulted in increased GM titers of anti-HSV-2 gE-specific IgG antibodies 22PII in all circumstances tested, relative to unadjuvanted control (FIG. 11 and FIG. 12).

Anti-HSV-2 gl-Specific IgG Antibodies

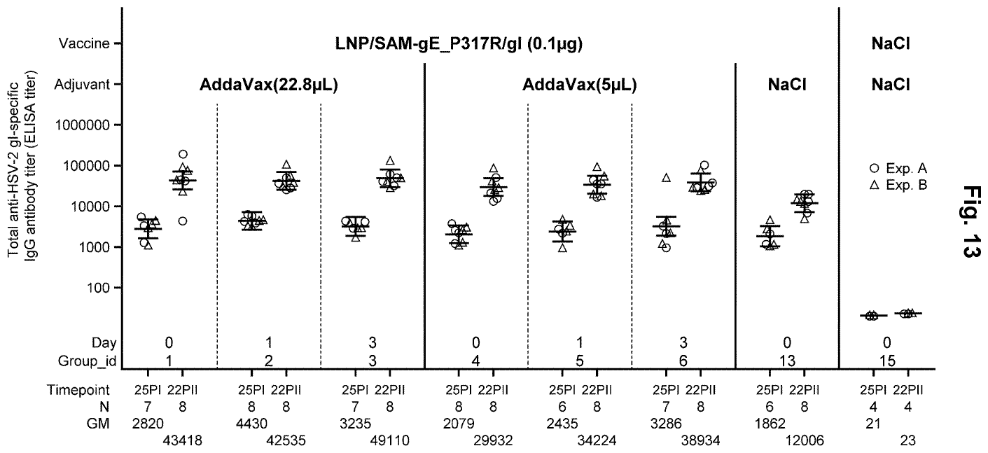

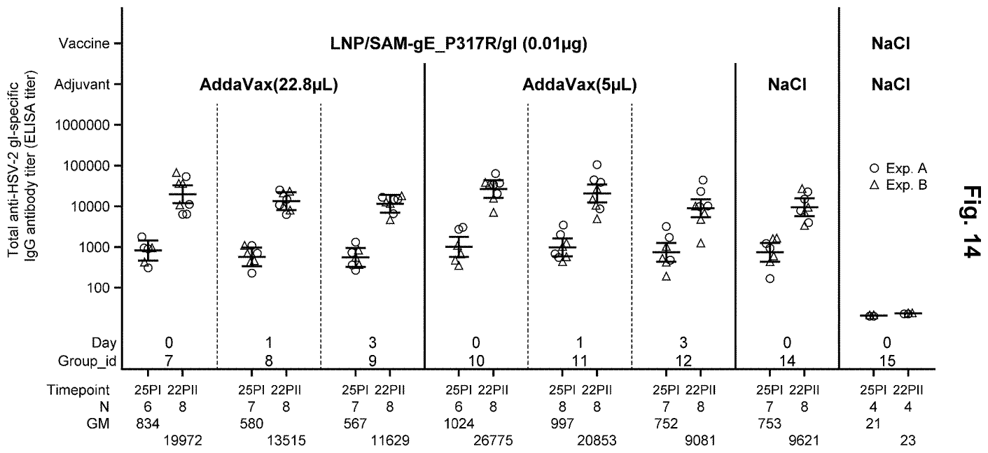

AddaVax™ adjuvanted 0.1 ug LNP/SAM-gE_P317/gl resulted in increased GM titers of anti-HSV-2 gl-specific IgG antibodies in all circumstances tested at 22PII, relative to unadjuvanted control (FIG. 13). Adjuvanted 0.01 ug LNP/SAM-gE_P317/gl resulted in increased GM titers of anti-HSV-2 gl-specific IgG antibodies in 5 of 6 circumstances tested at 22PII, relative to unadjuvanted control (FIG. 14).

### Evaluation of Vaccine-Specific Antibody Function 22 Days Post-Second Vaccination

Vaccine-specific antibody functions were investigated in the sera collected at 22 days post-second immunization by assaying the ability of polyclonal antibodies to decrease binding of human IgG (hlgG) Fc by HSV-2 gE/gl protein.

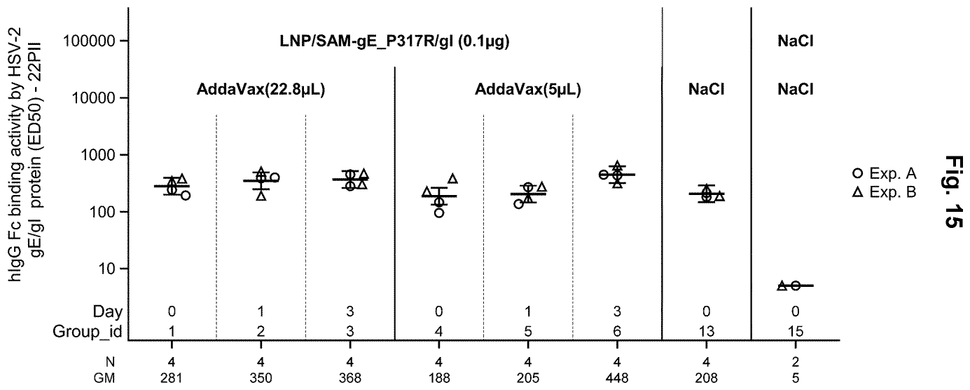

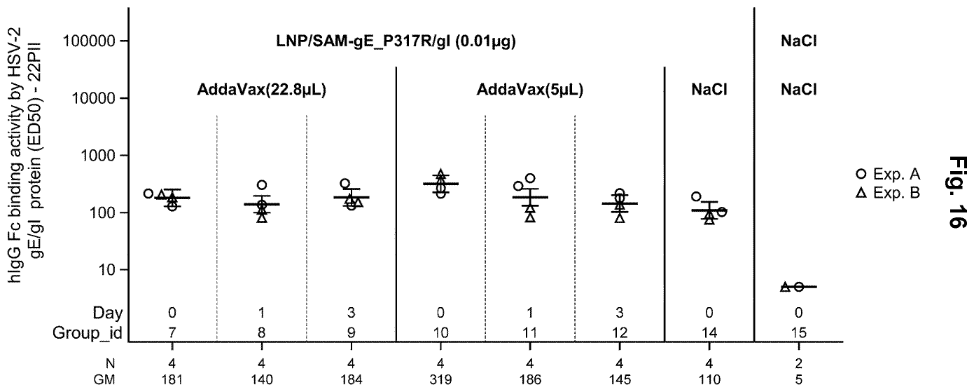

The adjuvanted 0.1 ug dose of LNP/SAM-gE_P317/gl resulted in increased GM ED50 values relative to unadjuvanted control when administered with 22.8 uL of adjuvant on all days (FIG. 15). The adjuvanted 0.01 ug dose of LNP/SAM-gE_P317/gl resulted in increased GM ED50 values relative to unadjuvanted control in all circumstances tested (FIG. 16).

### Evaluation of Vaccine-Specific T Cell Responses 22 Days Post-Second Vaccination

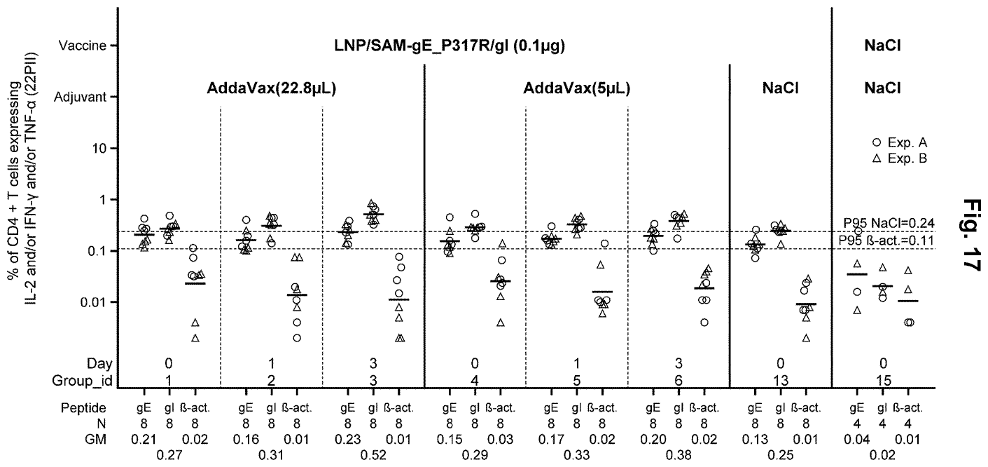

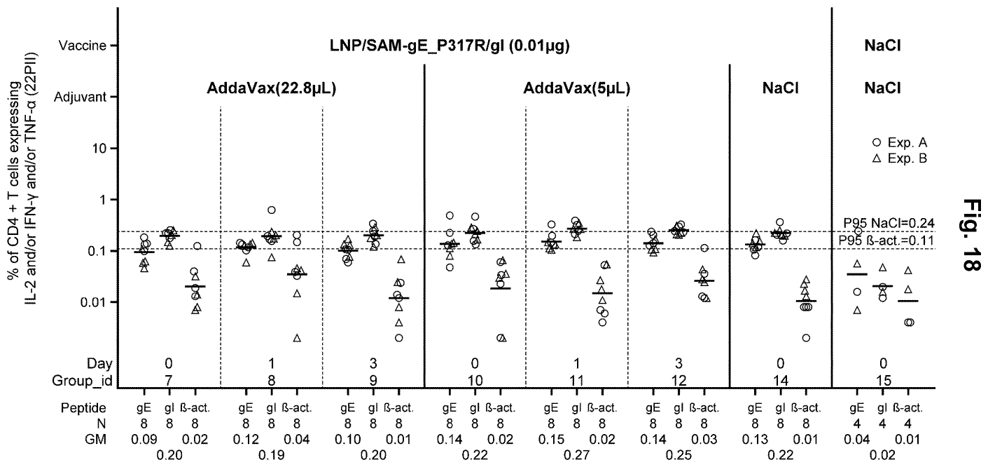

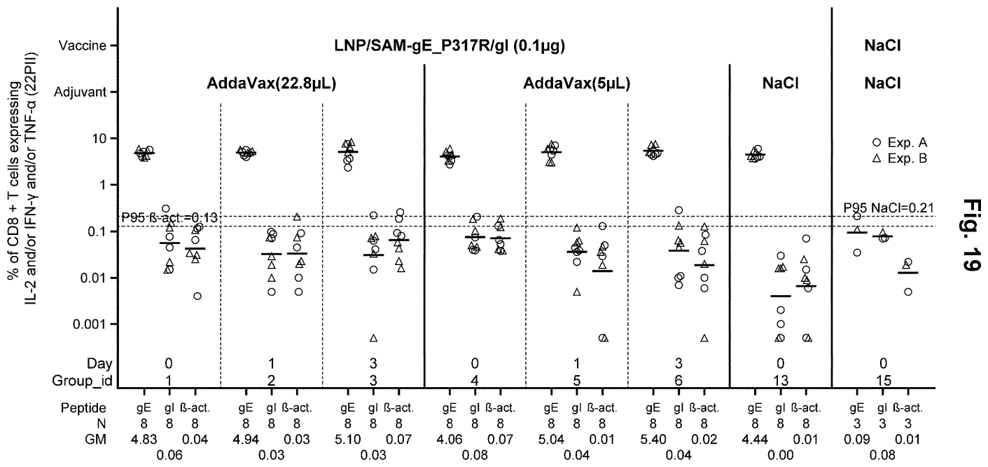

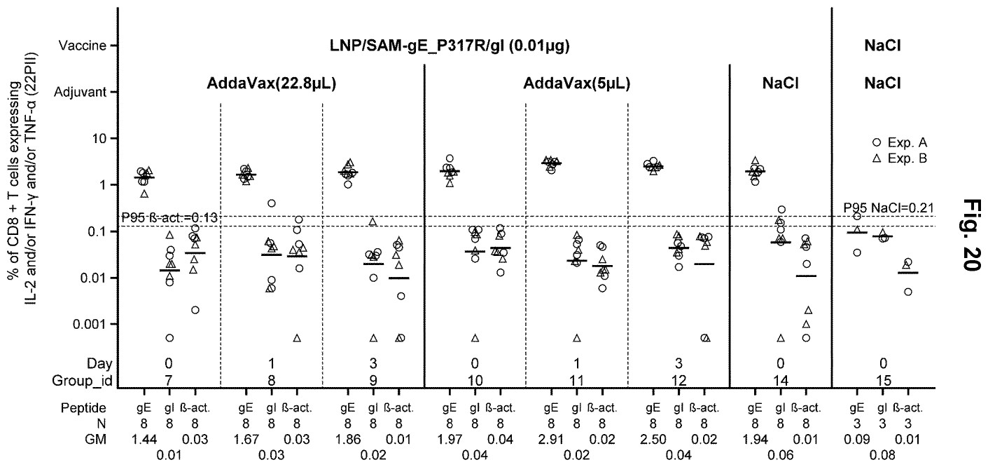

Twenty-two days after the second immunization (day 50), anti-HSV-2 gE & gl-specific CD4+/CD8+ T cell responses were measured in spleens from the immunized mice. Results are provided in FIGS. 17 to 20.

### Example 4—Immunogenicity Evaluation of Non-Replicating mRNA Administered with Squalene Emulsion Adjuvant in Mice

### Materials and Methods

### Mouse Immunisation, Vector Production and Vaccination Scheme

Female CB6F1 inbred mice aged 6-8 weeks old were randomly assigned to study groups (n=8 for groups 1-12 (gr1-12), n=16 for groups 12-14 (gr13-14) and n=4 for group 15 (gr15)) and kept under pathogen-free conditions. Mice (gr1-14) were intramuscularly (i.m.) immunized, in the gastrocnemius muscle, at days 0 & 21 with 0.8 ug of LNP-formulated mRNA ((SEQ ID No:11, non-modified; SEQ ID No:12, uridine replacement by N1-methyl-pseudouridine—‘N1mψ’)) encoding HSV-2 gE (SEQ ID No:13) (25 uL volume) and were injected, in the same muscle, with different doses of squalene emulsion adjuvant (AS03) prepared as detailed above under Example 1 (5 uL of emulsion, made up to 25 uL with PBS, or 25 uL of emulsion) either at the same time (day+0) or 1 or 3 days after each of the two mRNA immunizations. As positive controls for vaccine response, two groups of mice (gr13-14) were injected i.m. with a saline solution (NaCl 150 mM, 25 uL) at the same time as mRNA. Finally, as a negative control, mice (gr15) were i.m injected with only 50 uL of a saline solution (NaCl 150 mM), following the same schedule of immunization (see Table 4 for details of each group).

TABLE 4
Summary of the study design and formulations tested

| | | | Vaccine | Adjuvant | |
|---|---|---|---|---|---|
| | | | immunization | dose and | Volume and |
| | Number of | | schedule | immunization | route of |
| Group | animals | Vaccine name & dose | (Days) | schedule | injection |
| 1 | 8 | LNP/HSV-2 gE mRNA | Days 0 & 21 | 25 uL AS03 | i.m. |
| | | (native) | | Day + 0 | 25 uL each |
| 2 | 8 | LNP/HSV-2 gE mRNA | Days 0 & 21 | 25 uL AS03 | i.m. |
| | | (native) | | Day + 1 | 25 uL each |
| 3 | 8 | LNP/HSV-2 gE mRNA | Days 0 & 21 | 25 uL AS03 | i.m. |
| | | (native) | | Day + 3 | 25 uL each |
| 4 | 8 | LNP/HSV-2 gE mRNA | Days 0 & 21 | 5 uL AS03 | i.m. |
| | | (native) | | Day + 0 | 25 uL each |
| 5 | 8 | LNP/HSV-2 gE mRNA | Days 0 & 21 | 5 uL AS03 | i.m. |
| | | (native) | | Day + 1 | 25 uL each |
| 6 | 8 | LNP/HSV-2 gE mRNA | Days 0 & 21 | 5 uL AS03 | i.m. |
| | | (native) | | Day + 3 | 25 uL each |
| 7 | 8 | LNP/HSV-2 gE mRNA | Days 0 & 21 | 25 uL AS03 | i.m. |
| | | (N1mΨ) | | Day + 0 | 25 uL each |
| 8 | 8 | LNP/HSV-2 gE mRNA | Days 0 & 21 | 25 uL AS03 | i.m. |
| | | (N1mΨ) | | Day + 1 | 25 uL each |
| 9 | 8 | LNP/HSV-2 gE mRNA | Days 0 & 21 | 25 uL AS03 | i.m. |
| | | (N1mΨ) | | Day + 3 | 25 uL each |
| 10 | 8 | LNP/HSV-2 gE mRNA | Days 0 & 21 | 5 uL AS03 | i.m. |
| | | (N1mΨ) | | Day + 0 | 25 uL each |
| 11 | 8 | LNP/HSV-2 gE mRNA | Days 0 & 21 | 5 uL AS03 | i.m. |
| | | (N1mΨ) | | Day + 1 | 25 uL each |
| 12 | 8 | LNP/HSV-2 gE mRNA | Days 0 & 21 | 5 uL AS03 | i.m. |
| | | (N1mΨ) | | Day + 3 | 25 uL each |
| 13 | 16 | LNP/HSV-2 gE mRNA | Days 0 & 21 | (NaCl 150 mM) | i.m. |
| | | (native) | | Day + 0 | 25 uL each |
| 14 | 16 | LNP/HSV-2 gE mRNA | Days 0 & 21 | (NaCl 150 mM) | i.m. |
| | | (N1mΨ) | | Day + 0 | 25 uL each |
| 15 | 4 | NaCl 150 mM | Days 0 & 21 | (NaCl 150 mM) | i.m. |
| | | | | Day + 0 | 25 uL each |

Serum samples were collected at days 21 & 35 post-prime immunization (21 PI, 14PII) to measure anti-HSV-2 gE-specific and HSV-1 gE/gl cross-reactive IgG antibody responses and investigate the function of vaccine-specific antibodies. Spleens were also collected 35 days post-prime immunization (14PII) to evaluate anti-HSV-2 gE-specific and anti-HSV-1 gE/gl cross-reactive CD4+/CD8+ T cell responses. The study was split across two independent experiments—Experiment A (LIMS20210120) and Experiment B (LIMS20210122).

HSV gE non-replicating mRNA pDNA template was digested (linearized) with BspQ1 restriction enzyme, extracted with phenol: chloroform: isoamyl alcohol, washed with 70% ethanol, and resuspended in nuclease free water. In vitro transcription (IVT) reaction was performed with the linear template using a T7 Transcription kit at 37° C. for 4 hrs. IVT reactions contained either normal uridine or N1-Methyl-Pseudouridine-5′-Triphosphate. The IVT reactions were treated with TURBO DNase provided in the IVT kit, 15 minutes at 37° C., and the IVT product was lithium chloride purified and resuspended in nuclease free water. The IVT RNA was then capped using a m7G capping system with the addition of 2′-O-methyltransferase to achieve Cap-1 structure. The Capped IVT product was purified via silica column, and eluted in nuclease free water. The capped product was then treated with Anarctic phosphatase at 37° C. for 1 hr as per the manufacturer's recommendations. Finally, the Cap-1 IVT product was silica column purified as above and eluted in nuclease free water. The quality of the RNA was assessed by gel electrophoresis.

mRNA-LNP was prepared by rapidly mixing ethanolic solutions of lipids with aqueous buffers that contain the mRNA. The mRNA/lipid complexes are mixed to form lipid nanoparticles that entrap the RNA. Following this mixing step, the lipid nanoparticles matured to entrap the RNA and then were transferred to a final Tris/sucrose storage buffer through a buffer exchange step. The LNP solutions were then characterized for size, lipid content, RNA entrapment, final mRNA concentration, and in vitro potency. The LNP solutions comprised 20 mM Tris, 5 mM NaCl, 7.5% sucrose, and had a pH of 8.

### Detection of Total Anti-HSV-2 gE IgG Antibodies by ELISA

Quantification of the total anti-HSV-2 gE-specific IgG antibodies was performed using indirect ELISA. Recombinant HSV-2 gE (˜51 kDa) was used as coating antigen.

Polystyrene 96-well ELISA plates were coated with 100 uL/well of antigen diluted at a concentration of 2 ug/mL (HSV-2 gE) and 1 ug/mL (HSV-2 gl) in carbonate/bicarbonate 50 mM pH 9.5 buffer and incubated overnight at 4° C. After incubation, the coating solution was removed and the plates were blocked with 200 uL/well of skimmed milk media additive 10% diluted in PBS (blocking buffer) for 1 h at 37° C. The blocking solution was removed and three-fold sera dilutions (in PBS+0.1% Tween20+1% BSA buffer) were added to the coated plates and incubated for 1 h at 37° C. The plates were washed four times with PBS 0.1% Tween20 (washing buffer) and peroxidase conjugated goat anti-mouse IgG (H+L) was used as secondary antibody. One hundred microliters per well of the secondary antibody diluted at a concentration of 1:500 in PBS+0.1% Tween20+1% BSA buffer was added to each well and the plates were incubated for 45 min at 37° C.

The plates were then washed four times with washing buffer and 2 times with deionised water and incubated for 10 min at RT (room temperature) with 100 uL/well of a solution of 75% single-component TMB peroxidase ELISA substrate diluted in sodium citrate 0.1M pH5.5 buffer. Enzymatic color development was stopped with 100 uL of 0.4N sulfuric acid 1M (H₂SO₄) per well and the plates were read at an absorbance of 450/620 nm using an ELISA reader.

Optical densities (OD) were captured and analysed. A standard curve was generated by applying a 4-parameter logistic regression fit to a reference standard. Antibody titer in the samples was calculated by interpolation of the standard curve. The antibody titer of the samples was obtained by averaging the values from dilutions that fell within the 20-80% dynamic range of the standard curve. ELISA titers were normalized using a sample reference to allow titer comparisons.

Detection of Total Anti-HSV-1 gE/Gl Cross-Reactive IgG Antibodies Measured by ELISA

Quantification of the total anti-HSV-1 gE/gl-specific IgG antibodies was performed using indirect ELISA. Recombinant gE/gl heterodimer protein from HSV-1 was used as coating antigen. Polystyrene 96-well ELISA plates were coated with 100 μL/well of recombinant HSV-1 gE/gl protein diluted at a concentration of 2 pg/mL in carbonate/bicarbonate 50 mM pH 9.5 buffer and incubated overnight at 4° C. After incubation, the coating solution was removed and the plates were blocked with 200 μL/well of skimmed milk media additive 10% diluted in PBS (blocking buffer) for 1 h at 37° C. The blocking solution was removed and a three fold serial dilution of each sera was prepared (in PBS+0.1% Tween20+1% BSA buffer) and added to the coated plates for 1 h at 37° C. After incubation, the plates were washed four times with PBS 0.1% Tween20 (washing buffer) and 100 μl/well of peroxidase conjugated goat anti-mouse IgG (H+L) diluted at a concentration of 1:500 in PBS+0.1% Tween20+1% BSA buffer was added to each well for 45 min at 37° C. The plates were then washed four times with washing buffer and 2 times with deionised water and incubated for 10 min at RT (room temperature) with 100 μL/well of 75% single component TMB peroxidase ELISA substrate diluted in sodium citrate 0.1M pH5.5 buffer. Enzymatic colour development was stopped with 100 μL of 0.4N sulfuric acid 1M (H₂SO₄) per well and the plates were read at an absorbance of 450/620 nm using an ELISA reader. Optical densities (OD) were captured and analysed. A standard curve was generated by applying a 4-parameter logistic regression fit to a reference standard. Antibody titer in the samples was calculated by interpolation of the standard curve. The antibody titer of the samples was obtained by averaging the values from dilutions that fell within the 20-80% dynamic range of the standard curve. ELISA titers were normalized using a standard reference to allow titer comparisons.

Evaluation of Anti-HSV-2 gE-Specific and Anti-HSV-1 gE Cross-Reactive CD4+/CD8+ T Cell Responses by Intracellular Cytokine Staining (ICS)

The frequencies of HSV-2 gE-specific and HSV-1 gE cross-reactive CD4+ and CD8+ T-cells producing IL-2 and/or IFN-γ and/or TNF-α (Th1) and/or IL-17 (Th17) were evaluated in splenocytes collected 14 days second immunization after ex-vivo stimulation with HSV-2 gE or HSV-1 gE peptides pools.

### Isolation of Splenocytes

Spleens were collected from individual mice 14 days after second immunization and placed in RPMI 1640 medium supplemented with RPMI additives (glutamine, penicillin/streptomycin, sodium pyruvate, non-essential amino-acids & 2-mercaptoethanol) (=RPMI/additives). Cell suspensions were prepared from each spleen using a tissue grinder. The splenic cell suspensions were filtered (cell stainer 100 um) and then the filter was rinsed with 35 mL of cold PBS-EDTA 2 mM. After centrifugation (335 g, 10 min at 4° C.), cells were resuspended in 5 mL of cold PBS-EDTA. A second washing step was performed, as previously described, and the cells were finally resuspended in 2 mL of RPMI/additives supplemented with 5% FCS. Cell suspensions were then diluted 20× (10 uL) in PBS buffer (190 uL) for cell counting. After counting, cells were centrifuged (335 g, 10 min at RT) and resuspended at 10⁷ cells/mL in RPMI/additives supplemented with 5% FCS.

### Cell Preparation

Fresh splenocytes were seeded in round bottom 96-well plates at 10⁶ cells/well (100 uL). The cells were then stimulated for 6 hours (37° C., 5% CO₂) with anti-CD28 and anti-CD49d antibodies at 1 ug/mL per well, containing 100 uL of either:
15 mer overlapping peptide pool covering the sequences of gE protein from HSV-2 (1 ug/mL per peptide per well).
15 mer overlapping peptide pool covering the sequences of gE protein from HSV-1 (1 ug/mL per peptide per well).
15 mer overlapping peptide pool covering the sequences of human p-actin protein (1 ug/mL per peptide per well) (irrelevant stimulation).
RPMI/additives medium (as negative control of the assay).
PMA—ionomycin solution at working concentrations of 0.25 ug/mL and 2.5 ug/mL respectively (as positive control of the assay).

After 2 hours of ex vivo stimulation, brefeldin A protein transport inhibitor diluted 1/200 in RPMI/additives supplemented with 5% FCS was added for 4 additional hours to inhibit cytokine secretion. Plates were then transferred at 4° C. for overnight incubation.

### Intracellular Cytokine Staining

After overnight incubation at 4° C., cells were transferred to V-bottom 96-well plates, centrifuged (189 g, 5 min at 4° C.) and washed with 250 uL of cold PBS+1% FCS (flow buffer). After a second centrifugation (189 g, 5 min at 4° C.), cells were resuspended to block unspecific antibody binding (10 min at 4° C.) in 50 uL of flow buffer containing anti-CD16/32 antibodies diluted 1/50. Then, 50 uL flow buffer containing mouse anti-CD4-V700 antibodies, anti-CD8-PerCp-Cy5.5 antibodies and fixable yellow dead cell stain, anti-CD62L BV786, anti-CD127 BV421 was added for 30 min in darkness at 4° C. After incubation, 100 uL of flow buffer was added into each well and cells were then centrifuged (189 g for 5 min at 4° C.). A second washing step was performed with 200 uL of flow buffer and after centrifugation, cells were fixed and permeabilized by adding 200 uL of fixation solution for 20 min at 4° C. in darkness. After plate centrifugation (500 g for 5 min at 4° C.), cells were washed with 200 uL of cell wash buffer, centrifuged (500 g for 5 min 4° C.) and resuspended in 50 uL of cell wash buffer containing anti-IL2-FITC, anti-IFN-γ-APC and anti-TNF-α-PE, anti-IL-13 PE Cy7, anti-IL-17 BV605 antibodies, for 1 hour at 4° C. in darkness. After incubation, 100 uL of flow buffer was added into each well and cells were then finally washed with 200 uL of cell wash buffer (centrifugation 500 g for 5 min at 4° C.) and resuspended in 220 uL PBS.

### Cell Acquisition and Analysis

Stained cells were acquired by flow cytometry and analyzed. Live cells were identified with staining and then lymphocytes were isolated based on forward/side scatter lights (FSC/SSC) gating. The acquisition was performed on ˜20,000 CD4+/CD8+ T-cell events. The percentages of IFN-γ^{+/-}, IL-2^{+/-}, TNF-α^{+/-} and IL-17^{+/-} producing cells were calculated on CD4+ and CD8+ T cell populations (IL-13 was not analysed). For each sample, unspecific signal detected after medium stimulation was removed from the specific signal detected after peptide pool stimulation.

Competitive ELISA to Evaluate the Ability of Vaccine-Specific Antibodies to Decrease Human IgG Fc Binding by HSV-2 gE/Gl Protein

The ability of polyclonal sera collected in different groups of mice to decrease in-vitro hlgG Fc binding by recombinant HSV-2 gE/gl protein was investigated by competitive ELISA. Recombinant HSV-2 gE/gl protein was used as coating antigen.

Polystyrene 96-well ELISA plates were coated with 50 uL/well of HSV-2 gE/gl protein diluted at a concentration of 4 ug/mL in free calcium/magnesium PBS buffer and incubated overnight at 4° C. After incubation, the coating solution was removed and the plates were blocked with 100 uL/well of PBS supplemented with 0.1% Tween20+1% BSA for 1 h at 37° C.

Sixty microliters (60 uL) of two serial dilution of individual mouse serum (starting dilution 1/10 in blocking buffer) was prepared into 96-well clear V-bottom polypropylene microplate and mixed with 60 uL/well of biotinylated-hlgG antibodies pre-diluted at 0.7 ug/mL in blocking buffer. The blocking buffer from HSV-2 gE/gl coated plates was removed and 100 uL of the mixture was then transferred in the corresponding wells for an incubation period of 24 h at 37° C. Positive control serum of the assay was a pool of serum samples from mice immunized with HSV-2 gE/gl antigen in previous experiments. Negative control serum for the assay was a pool of irrelevant HPV serum samples diluted 1/1000 and mixed with hlgG.

After incubation, the plates were washed four times with PBS 0.1% Tween20 (washing buffer) and 50 uL/well of streptavidin-horseradish peroxidase diluted 2000× was added on the well and plates were incubated for 30 min at 37° C. After incubation, plates were washed four times with washing buffer and 50 uL/well of a solution containing 75% single-component TMB peroxidase ELISA substrate diluted in sodium citrate 0.1M pH5.5 buffer were added for 10 min at room temperature. Enzymatic color development was stopped with 50 uL/well of 0.4N sulfuric acid 1M (H₂SO₄) and the plates were read at an absorbance of 450/620 nm using an ELISA reader. Optical densities (OD) were captured and fitted to a curve. Titers were expressed as the effective dilution at which 50% (i.e. ED50) of the signal was achieved by sample dilution. For each plate and using a reference sample (i.e. irrelevant serum), the reference ED50 value was estimated using the following formula:

ED50=OD_{0%}+0.5*(OD_{100%}−OD_{0%})

where OD_{100%} is the highest OD obtained with similar samples and OD_{0%} is the lowest achievable signal. For each plate, the former was obtained by averaging (mean) 6 replicates while the latter was set at zero. The samples' ED50 titers were computed by way of linear interpolation between the left and right measurements closest to the ED50 estimate within the plate. The approximation was obtained, on the untransformed OD and the logarithm base 10 transformed dilutions.

Samples were not assigned a titer in the following cases:
no measurement was available above or below the ED50,
curve crossed at least twice the ED50 and
one of the dilution step (left or right) closest to the ED50 was missing.

### Statistical Analysis

For anti-HSV-2 gE-specific IgG antibody responses, an analysis of variance model for repeated measures was fitted on log 10 titers by including Group (all groups except NaCl group 15), Timepoint (PI and P1l), and Group x Timepoint interaction as fixed effects. The variance-covariance structure related to both timepoints was modelled via an Unstructured matrix, which considers different variances at PI and PII. This structure was estimated differently for each day x adjuvant condition, indicating different variances and timepoint correlations between them. Geometric means and their 95% CIs were derived from this model as well as geometric mean ratios and their 90% CIs.

For cross-reactive IgG antibody titers, an analysis of variance model was fitted on log 10 anti-HSV-1 gE/gl cross-reactive IgG antibody response by including Group (all groups except NaCl group 15) as fixed effect. Different variances were modeled between day x adjuvant conditions, as heterogeneity of variance was detected between them. Geometric means and their 95% CIs were derived from this model as well as geometric mean ratios and their 90% CIs of adjuvanted over unadjuvanted groups.

For hlgG Fc binding by HSV-2 gE/gl protein (PII), an analysis of variance model was fitted on log 10 ED50 by including Group (all groups except NaCl group 15) and Experiment as fixed effects. No clear heterogeneity of variance was detected and therefore identical variances were assumed for the different groups. Geometric means and their 95% CIs were derived from this model as well as geometric mean ratios and their 90% CIs of adjuvanted over unadjuvanted groups. The impact of Experiment on group effects was small.

For T cell responses, the NaCl P95 threshold is the 95^{th} percentile on % of CD4+ or CD8+ T cell data across HSV-2 & HSV-1 gE stimulation in NaCl negative control group 15. For information, the β-actin P95 threshold was also computed based on data from all groups. For each response (% of anti-HSV-2 gE-specific or anti-HSV-1 gE cross-reactive CD4+ or CD8+ T cell), an analysis of variance (ANOVA) model is fitted on log 10 values by including group (all groups except NaCl group 15) as fixed effect. For models on % of anti-HSV-2 gE-specific CD4+& CD8+ T cell, different variances were assumed for the different groups. For the model on % of anti-HSV-1 gE cross-reactive CD4+ T cell, different variances were assumed for the two vaccine formulations. Finally, for the model on % of anti-HSV-1 gE cross-reactive CD8+ T cell, different variances were modeled between day x adjuvant conditions. Geometric means and their 95% CIs are derived from these models as well as geometric mean ratios and their 90% CIs of adjuvanted over unadjuvanted groups.

### Results

### Evaluation of Vaccine-Specific Antibody Responses

The anti-HSV-2 gE-specific IgG antibody response was investigated by ELISA at day 21 (21PI) and day 35 (14PII) while the anti-HSV-1 gE/gl cross-reactive IgG antibody response was only investigated at day 35 (14PII). Results are expressed in ELISA titers and normalized using a sample reference to allow titers comparisons between groups.

Anti-HSV-2 gE-Specific IgG Antibodies

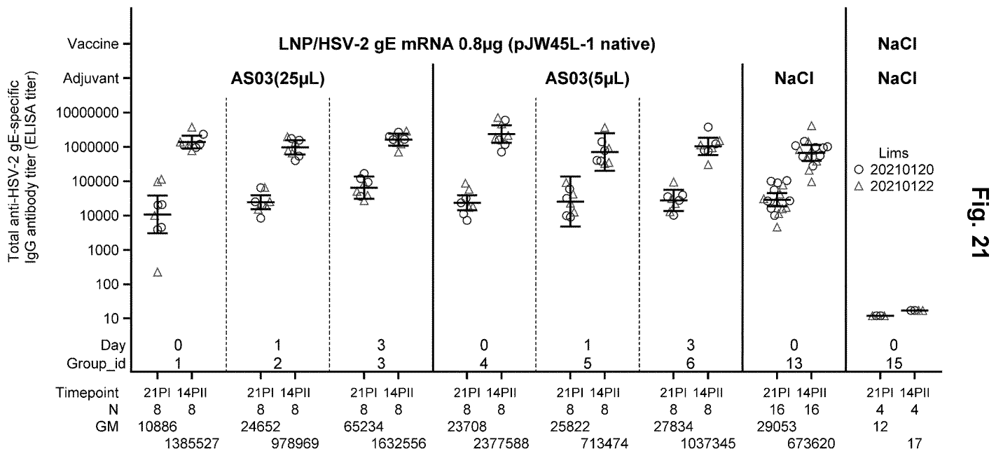

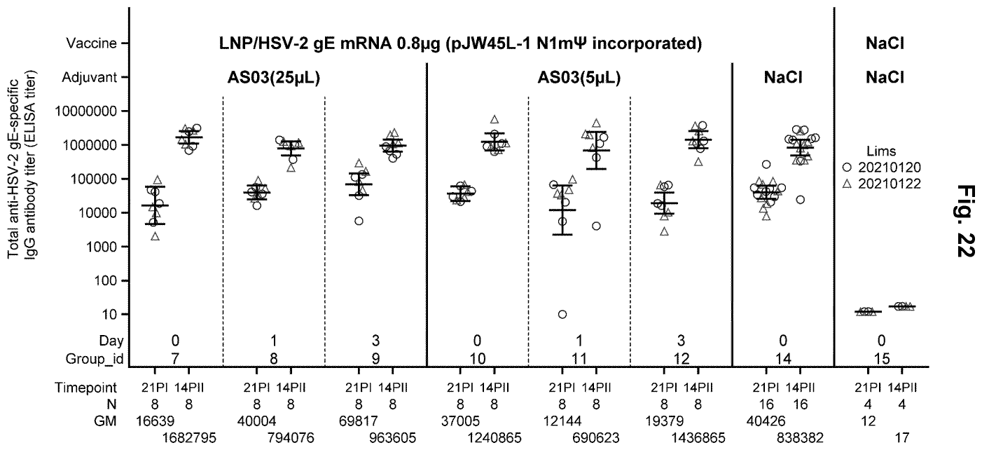

The results for anti-HSV-2 gE-specific IgG antibodies are shown in FIG. 21 (native mRNA) and FIG. 22 (N1mψ).

In all circumstances tested, after the second vaccination (14PII) AS03 adjuvanted LNP/mRNA (native) resulted in increased GM titers of anti-HSV-2 gE-specific IgG antibodies, relative to unadjuvanted control.

Statistical analysis found higher anti-HSV-2 gE-specific IgG antibody response seemed to be observed in the group of mice immunized with native LNP/HSV-2 gE mRNA vaccine and administered simultaneously (at day 0) with low dose of AS03 (5 μL) compared to the corresponding unadjuvanted-mRNA vaccinated group of mice (GMR of 3.53 with CI not containing 1). In addition, higher anti-HSV-2 gE-specific IgG antibody response was also observed in mice that received the highest dose of AS03 (25 μL) co-administered at day 3 post immunization with the native LNP/HSV-2 gE mRNA vaccine compared to the unadjuvanted group of mice receiving the same vaccine dose (GMR of 2.42, CI not containing 1). Finally, higher anti-HSV-2 gE-specific IgG antibody response seemed to be observed in groups of mice immunized with both native and chemically modified (N1mψ incorporation) LNP/HSV-2 gE mRNA vaccines and administered simultaneously (at day 0) with the highest dose of AS03 (25 μL) compared to the corresponding unadjuvanted-mRNA vaccinated group of mice (GMRs of 2.01-2.06 with CIs not containing 1).

Evaluation of Total Anti-HSV-1 gE/Gl Cross-Reactive IgG Antibodies Measured by ELISA

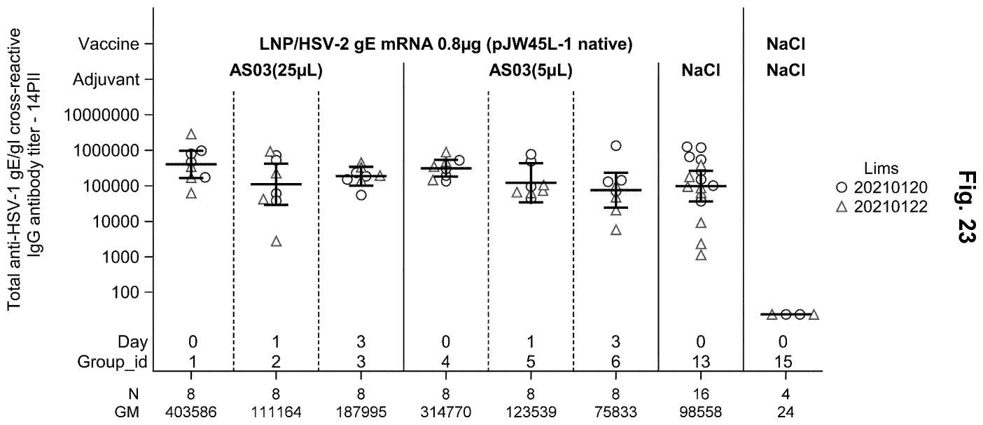

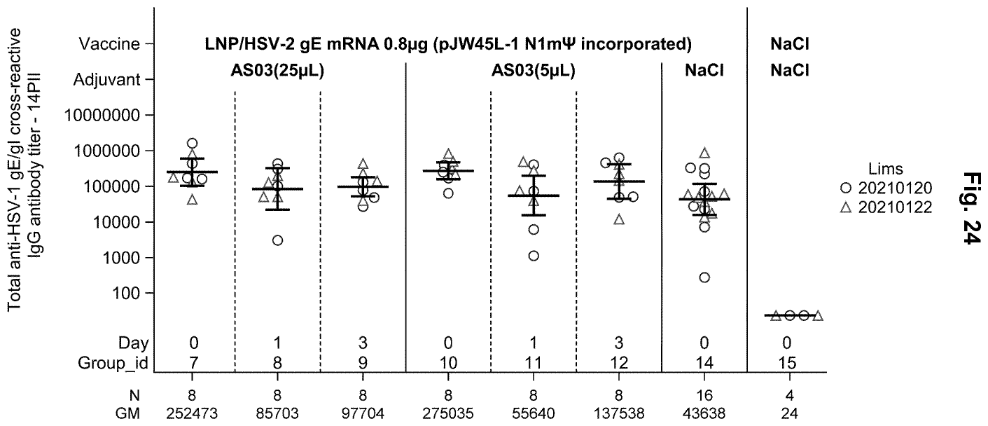

The results for anti-HSV-1 gE/gl cross-reactive IgG antibodies are shown in FIG. 23 (native mRNA) and FIG. 24 (N1mψ).

At 14 days post second immunization, higher anti-HSV-1 gE/gl cross-reactive IgG antibody response seemed to be observed in both groups of mice immunized with native or chemically modified LNP/HSV-2 gE mRNA vaccine and administered simultaneously (at day 0) with both doses of AS03 (25 or 5 μL) compared to the corresponding unadjuvanted-mRNA vaccinated group of mice (GMRs of 3.19-6.30 with CIs not containing 1).

### Evaluation of Vaccine-Specific Antibody Function 14 Days Post-Second Vaccination

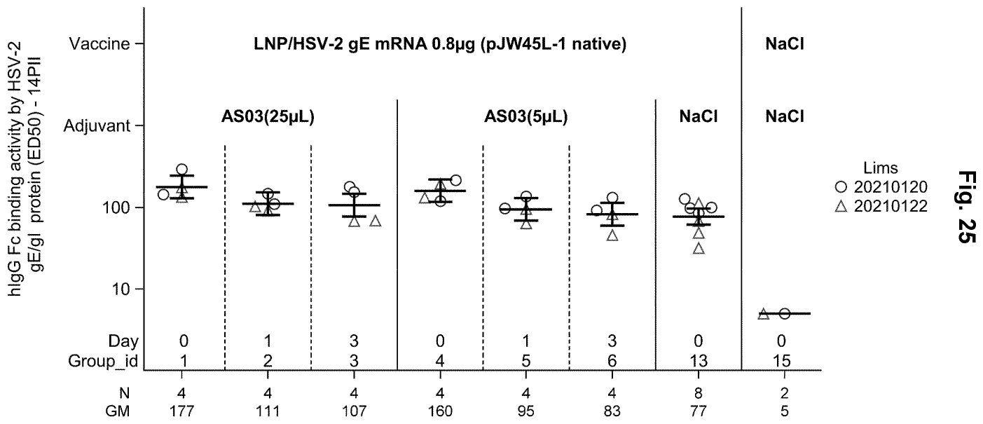

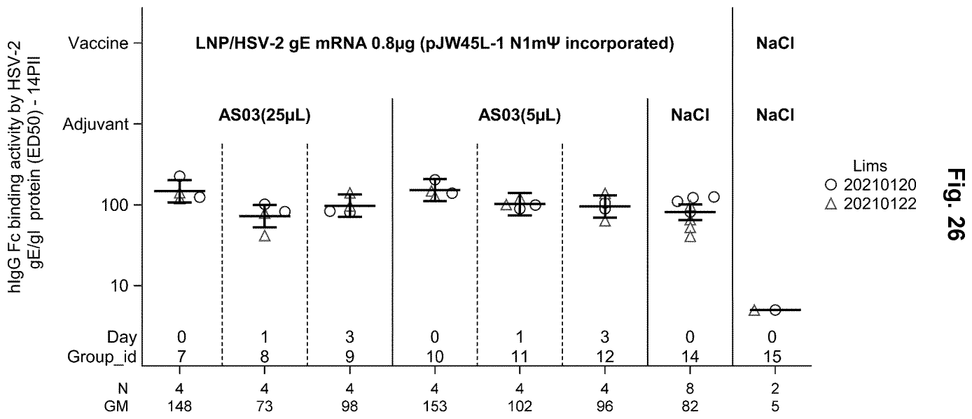

Vaccine-specific antibody functions were investigated in the sera collected at 14 days post-second immunization by assaying the ability of polyclonal antibodies to decrease binding of human IgG (hlgG) Fc by HSV-2 gE/gl protein. Results are shown in FIG. 25 (native mRNA) and FIG. 26 (N1mψ).

In all circumstances tested, after the second vaccination (14PII) AS03 adjuvanted LNP/mRNA (native) resulted in increased GM ED50, relative to unadjuvanted control.

### Evaluation of Vaccine-Specific T Cell Responses 14 Days Post-Second Vaccination

Fourteen days after the second immunization (day 35), the anti-HSV-2 gE-specific and anti-HSV-1 gE cross-reactive CD4+/CD8+ T cell responses were measured for all mice enrolled in this study in the spleen. Note that due to technical issues, no data was available for one mouse in gr14 for HSV-2 gE, HSV-1 gE and p-actin peptides pools stimulations.

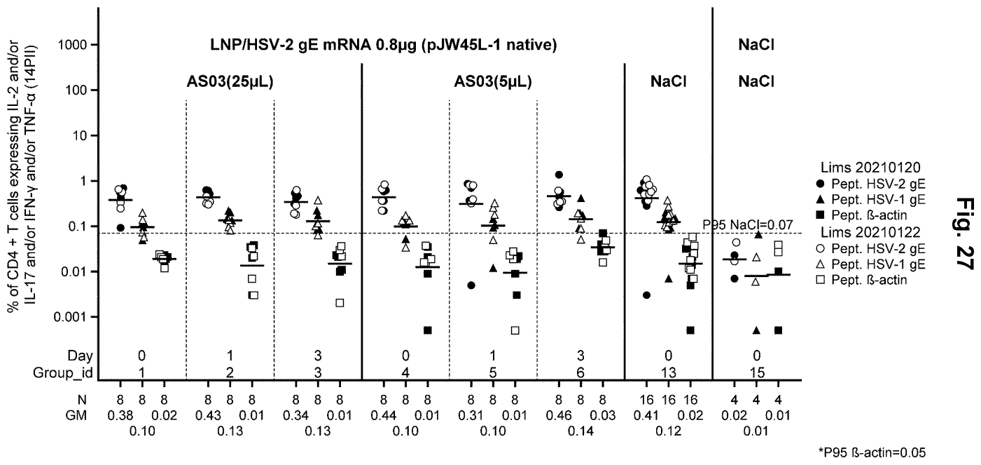

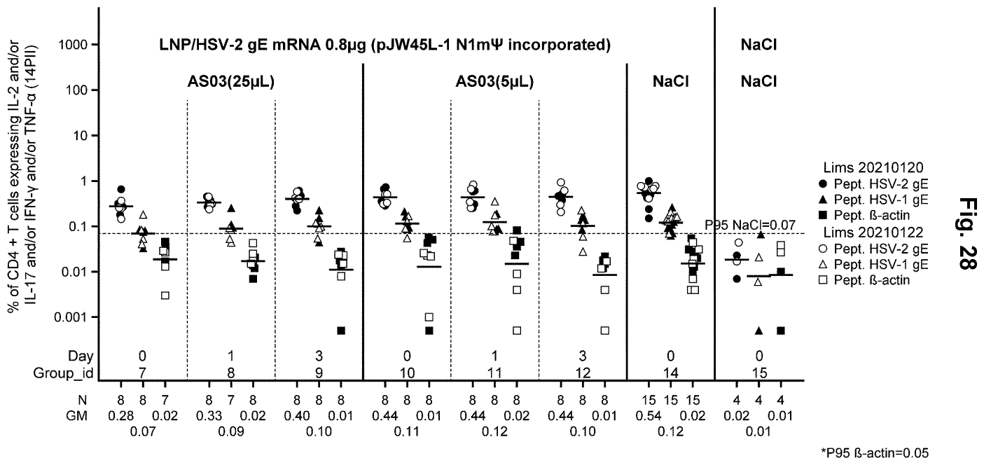

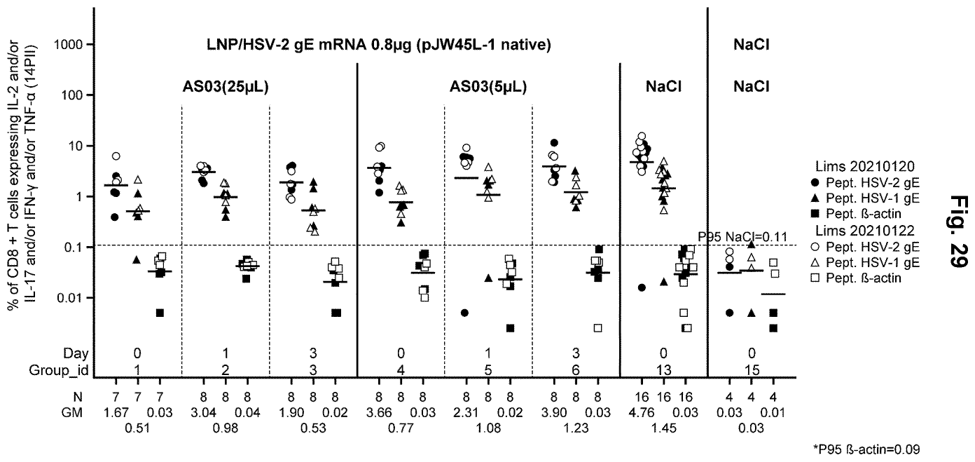

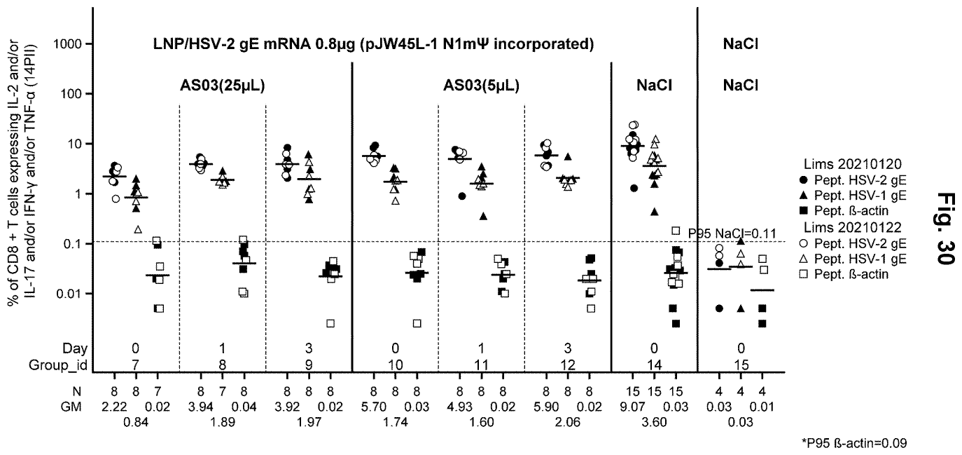

Results are provided in FIGS. 27 to 30.

### Example 5—Immunogenicity Evaluation of Non-Replicating mRNA Administered with a Different Squalene Emulsion Adjuvant in Mice

### Materials and Methods

### Mouse Immunisation, Vector Production and Vaccination Scheme

Female CB6F1 inbred mice aged 6-8 weeks old were randomly assigned to study groups (n=8 for groups 1-12 (gr1-12), n=16 for groups 12-14 (gr13-14) and n=4 for group 15 (gr15)) and kept under pathogen-free conditions. Mice (gr-14) were intramuscularly (i.m.) immunized, in the gastrocnemius muscle, at days 0 & 21 with (0.8 ug of LNP-formulated mRNA ((SEQ ID No: 11, non-modified; SEQ ID No: 12, uridine replacement by N1-methyl-pseudouridine—‘N1mψ’)) encoding HSV-2 gE (SEQ ID No: 13) (25 uL volume) and were injected, in the same muscle, with different doses of squalene emulsion adjuvant (AddaVax) as detailed above under Example 3 (5 uL of emulsion or 22.8 uL of emulsion, each made up to 25 uL with PBS) either at the same time (day+0) or 1 or 3 days after each of the two mRNA immunizations. As positive controls for vaccine response, two groups of mice (gr13-14) were injected i.m. with a saline solution (NaCl 150 mM, 25 uL) at the same time as mRNA. Finally, as a negative control, mice (gr15) were i.m injected with only 50 uL of a saline solution (NaCl 150 mM), following the same schedule of immunization (see Table 5 for details of each group).

TABLE 5
Summary of the study design and formulations tested

| | | | Vaccine | Adjuvant | |
|---|---|---|---|---|---|
| | | | immunization | dose and | Volume and |
| | Number of | | schedule | immunization | route of |
| Group | animals | Vaccine name & dose | (Days) | schedule | injection |
| 1 | 8 | LNP/HSV-2 gE mRNA | Days 0 & 21 | AddaVax | i.m. |
| | | (native) | | (22.8 μL) | 25 uL each |
| | | | | Day + 0 | |
| 2 | 8 | LNP/HSV-2 gE mRNA | Days 0 & 21 | AddaVax | i.m. |
| | | (native) | | (22.8 μL) | 25 uL each |
| | | | | Day + 1 | |
| 3 | 8 | LNP/HSV-2 gE mRNA | Days 0 & 21 | AddaVax | i.m. |
| | | (native) | | (22.8 μL) | 25 uL each |
| | | | | Day + 3 | |
| 4 | 8 | LNP/HSV-2 gE mRNA | Days 0 & 21 | AddaVax | i.m. |
| | | (native) | | (5 μL) | 25 uL each |
| | | | | Day + 0 | |
| 5 | 8 | LNP/HSV-2 gE mRNA | Days 0 & 21 | AddaVax | i.m. |
| | | (native) | | (5 μL) | 25 uL each |
| | | | | Day + 1 | |
| 6 | 8 | LNP/HSV-2 gE mRNA | Days 0 & 21 | AddaVax | i.m. |
| | | (native) | | (5 μL) | 25 uL each |
| | | | | Day + 3 | |
| 7 | 8 | LNP/HSV-2 gE mRNA | Days 0 & 21 | AddaVax | i.m. |
| | | (N1mΨ) | | (22.8 μL) | 25 uL each |
| | | | | Day + 0 | |
| 8 | 8 | LNP/HSV-2 gE mRNA | Days 0 & 21 | AddaVax | i.m. |
| | | (N1mΨ) | | (22.8 μL) | 25 uL each |
| | | | | Day + 1 | |
| 9 | 8 | LNP/HSV-2 gE mRNA | Days 0 & 21 | AddaVax | i.m. |
| | | (N1mΨ) | | (22.8 μL) | 25 uL each |
| | | | | Day + 3 | |
| 10 | 8 | LNP/HSV-2 gE mRNA | Days 0 & 21 | AddaVax | i.m. |
| | | (N1mΨ) | | (5 μL) | 25 uL each |
| | | | | Day + 0 | |
| 11 | 8 | LNP/HSV-2 gE mRNA | Days 0 & 21 | AddaVax | i.m. |
| | | (N1mΨ) | | (5 μL) | 25 uL each |
| | | | | Day + 1 | |
| 12 | 8 | LNP/HSV-2 gE mRNA | Days 0 & 21 | AddaVax | i.m. |
| | | (N1mΨ) | | (5 μL) | 25 uL each |
| | | | | Day + 3 | |
| 13 | 16 | LNP/HSV-2 gE mRNA | Days 0 & 21 | (NaCl 150 mM) | i.m. |
| | | (native) | | Day + 0 | 25 uL each |
| 14 | 16 | LNP/HSV-2 gE mRNA | Days 0 & 21 | (NaCl 150 mM) | i.m. |
| | | (N1mΨ) | | Day + 0 | 25 uL each |
| 15 | 4 | NaCl 150 mM | Days 0 & 21 | (NaCl 150 mM) | i.m. |
| | | | | Day + 0 | 25 uL each |

Serum samples were collected at days 21 & 35 post-prime immunization (21 PI, 14PII) to measure anti-HSV-2 gE-specific and HSV-1 gE/gl cross-reactive IgG antibody responses and investigate the function of vaccine-specific antibodies. Spleens were also collected 35 days post-prime immunization (14PII) to evaluate anti-HSV-2 gE-specific and anti-HSV-1 gE/gl cross-reactive CD4+/CD8+ T cell responses. The study was split across two independent experiments—Experiment A (LIMS20210123) and Experiment B (LIMS20210124).

mRNA was prepared as described in Example 4.

mRNA-LNP was prepared by rapidly mixing ethanolic solutions of lipids with aqueous buffers that contain the mRNA. The mRNA/lipid complexes are mixed to form lipid nanoparticles that entrap the RNA. Following this mixing step, the lipid nanoparticles matured to entrap the RNA and then were transferred to a final Tris/sucrose storage buffer through a buffer exchange step. The LNP solutions were then characterized for size, lipid content, RNA entrapment, final mRNA concentration, and in vitro potency. The LNP solutions comprised 20 mM Tris, 5 mM NaCl, 7.5% sucrose, and had a pH of 8.

### Detection of Total Anti-HSV-2 gE IgG Antibodies by ELISA

Performed as described in Example 4.

Detection of Total Anti-HSV-1 gE/Gl Cross-Reactive IgG Antibodies Measured by ELISA

Performed as described in Example 4.

Evaluation of Anti-HSV-2 gE-Specific and Anti-HSV-1 gE Cross-Reactive CD4+/CD8+ T Cell Responses by Intracellular Cytokine Staining (ICS)

The frequencies of HSV-2 gE-specific and HSV-1 gE cross-reactive CD4+ and CD8+ T-cells producing IL-2 and/or IFN-γ and/or TNF-α (Th1) and/or IL-17 (Th17) were evaluated in splenocytes collected 14 days second immunization after ex-vivo stimulation with HSV-2 gE or HSV-1 gE peptides pools.

Performed as described in Example 4.

### Statistical Analysis

For anti-HSV-2 gE-specific IgG antibody responses, an analysis of variance model for repeated measures was fitted on log 10 titers by including Group (all groups except NaCl group 15), Timepoint (PI and P1l), and Group x Timepoint interaction as fixed effects. The variance-covariance structure related to both timepoints was modelled via an Unstructured matrix, which considers different variances at PI and PII. This structure was estimated differently for each day x adjuvant condition, indicating different variances and timepoint correlations between them. Geometric means and their 95% CIs were derived from this model as well as geometric mean ratios and their 90% CIs.

For cross-reactive IgG antibody titers, an analysis of variance model was fitted on log 10 anti-HSV-1 gE/gl cross-reactive IgG antibody response by including Group (all groups except NaCl group 15) as fixed effect. No clear heterogeneity of variance was detected and therefore identical variances were assumed for the different groups. Geometric means and their 95% CIs were derived from this model as well as geometric mean ratios and their 90% CIs of adjuvanted over unadjuvanted groups.

For hlgG Fc binding by HSV-2 gE/gl protein (P1l), an analysis of variance model was fitted on log 10 ED50 by including Group (all groups except NaCl group 15) and Experiment as fixed effects. No clear heterogeneity of variance was detected and therefore identical variances were assumed for the different groups. Geometric means and their 95% CIs were derived from this model as well as geometric mean ratios and their 90% CIs of adjuvanted over unadjuvanted groups.

For T cell responses, the NaCl P95 threshold is the 95^{th} percentile on % of CD4+ or CD8+ T cell data across HSV-2 & HSV-1 gE stimulation in NaCl negative control group 15. For information, the R-actin P95 threshold was also computed based on data from all groups. For % of anti-HSV-2 gE-specific CD4+ or CD8+ T cell, or anti-HSV-1 gE cross-reactive CD4+ T cell, an analysis of variance (ANOVA) model is fitted on log 10 values by including group (all groups except NaCl group 15), experiment (Lims) and Group x experiment (Lims) interaction as fixed effects. For % of anti-HSV-1 gE cross-reactive CD8+ T cell, an analysis of variance (ANOVA) model is fitted on log 10 values by including Group (all groups except NaCl group 15) and experiment (Lims) as fixed effects. For the model on % of anti-HSV-2 gE-specific CD4+ T cell, no clear heterogeneity of variance was detected and therefore identical variances were assumed for the different groups. For the model on % of anti-HSV-2 gE-specific CD8+ T cell, different variances were modelled between adjuvant conditions. For models on % of anti-HSV-1 gE cross-reactive CD4/CD8+ T cell, different variances were modelled between day x adjuvant conditions. Geometric means and their 95% CIs are derived from these models as well as geometric mean ratios and their 90% CIs of adjuvanted over unadjuvanted groups. The impact of experiment (Lims) on group effects seemed small.

### Results

### Evaluation of Vaccine-Specific Antibody Responses

The anti-HSV-2 gE-specific IgG antibody response was investigated by ELISA at day 21 (21PI) and day 35 (14PII) while the anti-HSV-1 gE/gl cross-reactive IgG antibody response was only investigated at day 35 (14PII). Results are expressed in ELISA titers and normalized using a sample reference to allow titers comparisons between groups.

Anti-HSV-2 gE-Specific IgG Antibodies

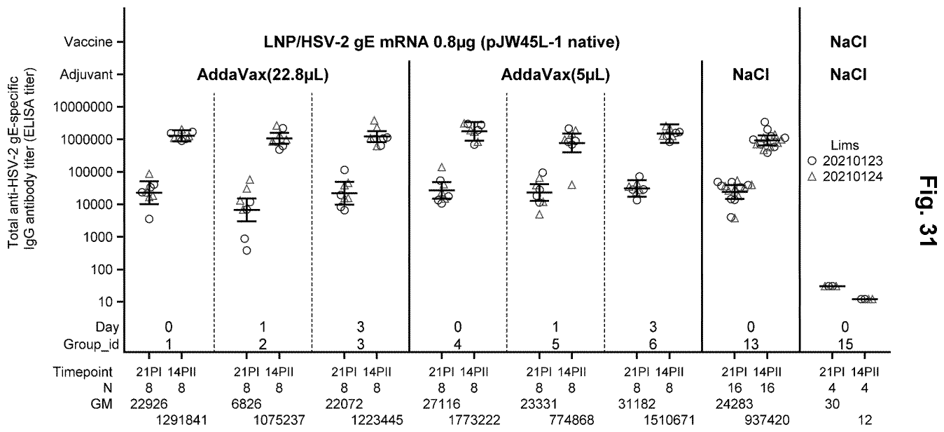

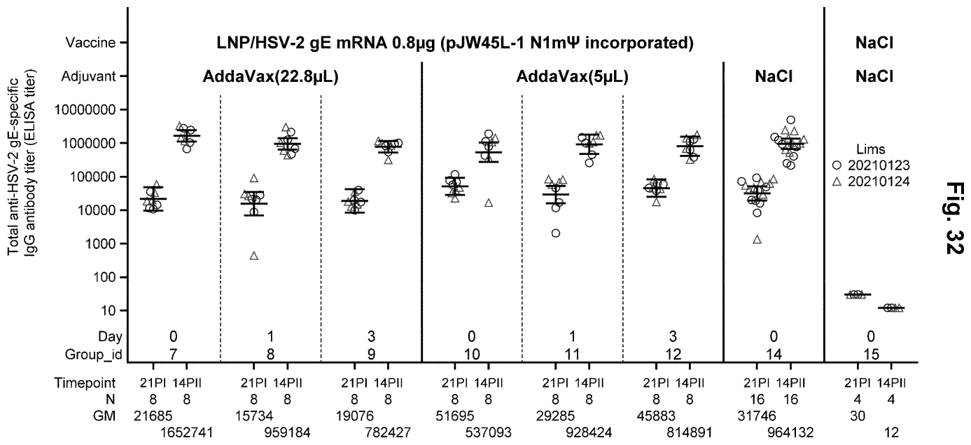

The results for anti-HSV-2 gE-specific IgG antibodies are shown in FIG. 31 (native mRNA) and FIG. 32 (N1mψ).

After the second vaccination (14PII) for all three dose timings, 22.8 uL of AddaVax adjuvanted LNP/mRNA (native) resulted in increased GM titers of anti-HSV-2 gE-specific IgG antibodies, relative to unadjuvanted control.

Statistical analysis found higher anti-HSV-2 gE-specific IgG antibody response seemed to be observed in the group of mice immunized with native LNP/HSV-2 gE mRNA vaccine and administered simultaneously (at day 0) with low dose of AddaVax (5 μL) compared to the corresponding unadjuvanted-mRNA vaccinated group of mice (GMR of 1.89 with CI not containing 1). Similarly, higher anti-HSV-2 gE-specific IgG antibody response seemed to be observed in mice immunized with chemically modified LNP/HSV-2 gE mRNA vaccine and administered simultaneously (at day 0) with a high dose of AddaVax (22.8 μL) compared to the corresponding unadjuvanted-mRNA vaccinated group of mice (GMR of 1.71 with CI not containing 1). Note that both positive effects were statistically significant (CIs not containing 1), although limited in magnitude (GMRs <2).

Evaluation of Total Anti-HSV-1 gE/Gl Cross-Reactive IgG Antibodies Measured by ELISA

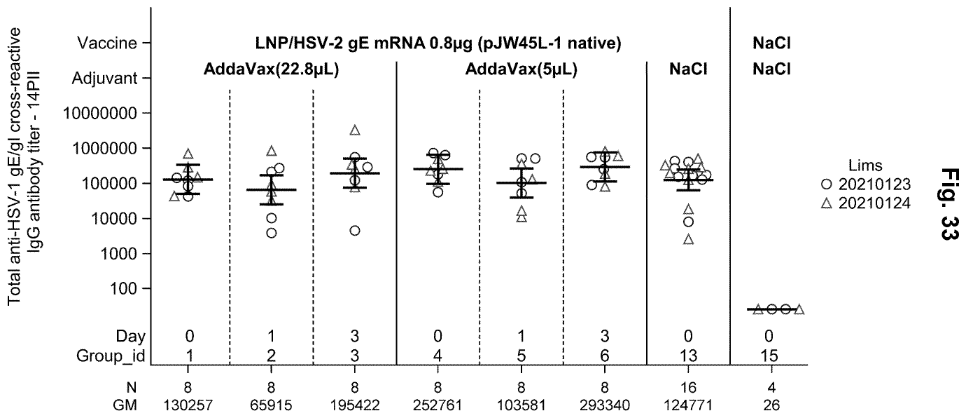

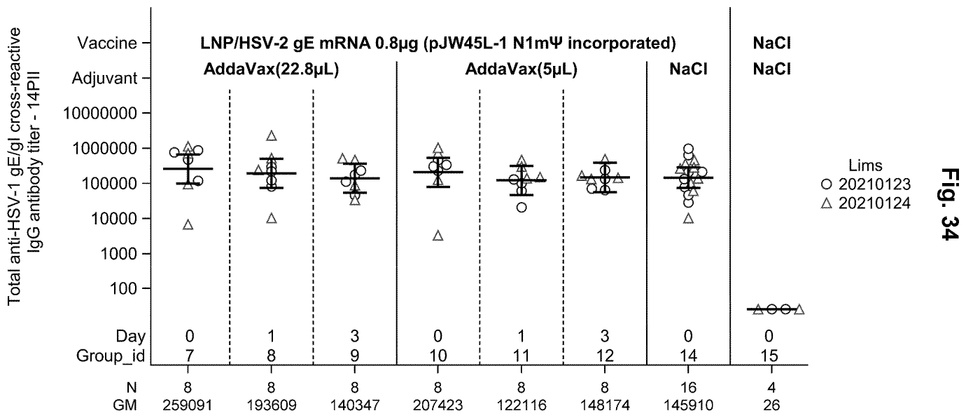

The results for anti-HSV-1 gE/gl cross-reactive IgG antibodies are shown in FIG. 33 (native mRNA) and FIG. 34 (N1mψ).

At 14 days post second immunization, higher anti-HSV-1 gE/gl cross-reactive IgG antibody response was observed in 8 of 12 groups of mice immunized with native or chemically modified LNP/HSV-2 gE mRNA vaccine and administered with both doses of AddaVax (25 or 5 μL) compared to the corresponding unadjuvanted-mRNA vaccinated group of mice.

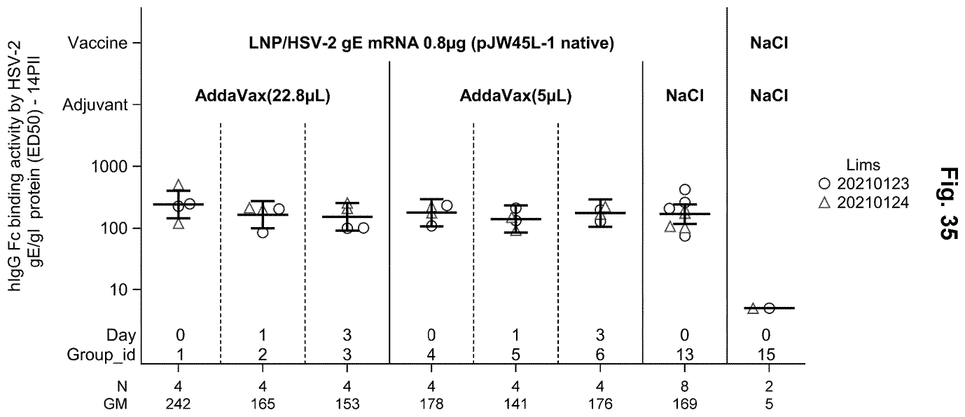

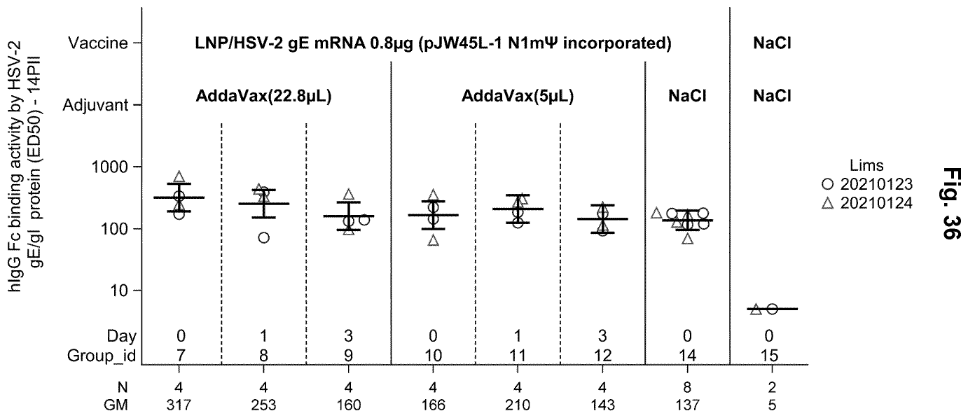

Evaluation of vaccine-specific antibody function 14 days post-second vaccination Vaccine-specific antibody functions were investigated in the sera collected at 12 days post-second immunization by assaying the ability of polyclonal antibodies to decrease binding of human IgG (hlgG) Fc by HSV-2 gE/gl protein. Results are shown in FIG. 35 (native mRNA) and FIG. 36 (N1mψ).

### Evaluation of Vaccine-Specific T Cell Responses 14 Days Post-Second Vaccination

Fourteen days after the second immunization (day 35), the anti-HSV-2 gE-specific and anti-HSV-1 gE cross-reactive CD4+/CD8+ T cell responses were measured for all mice enrolled in this study in the spleen.

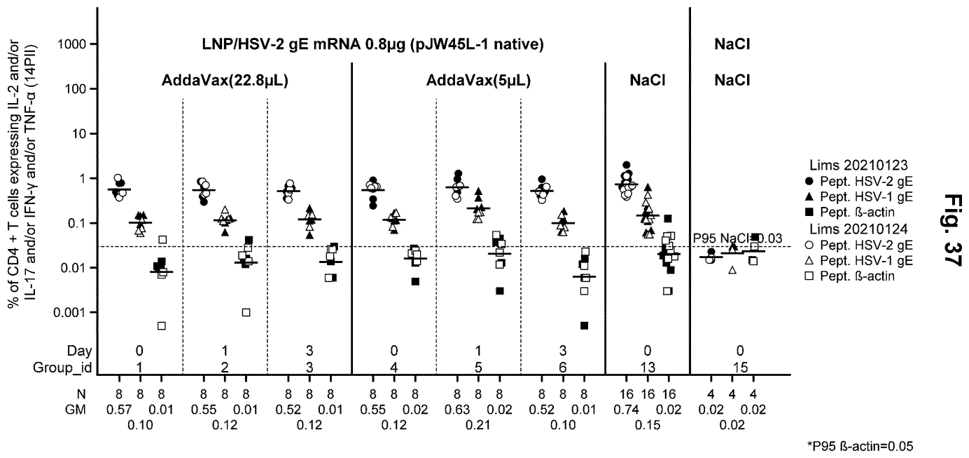

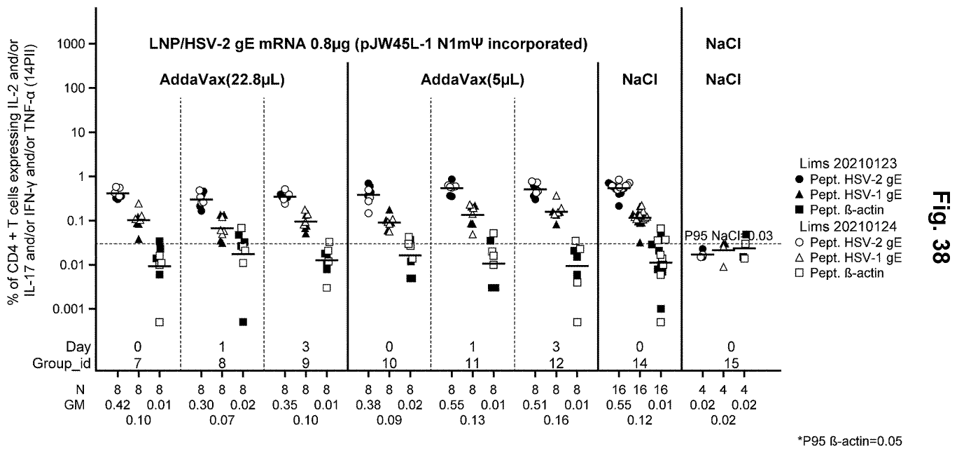

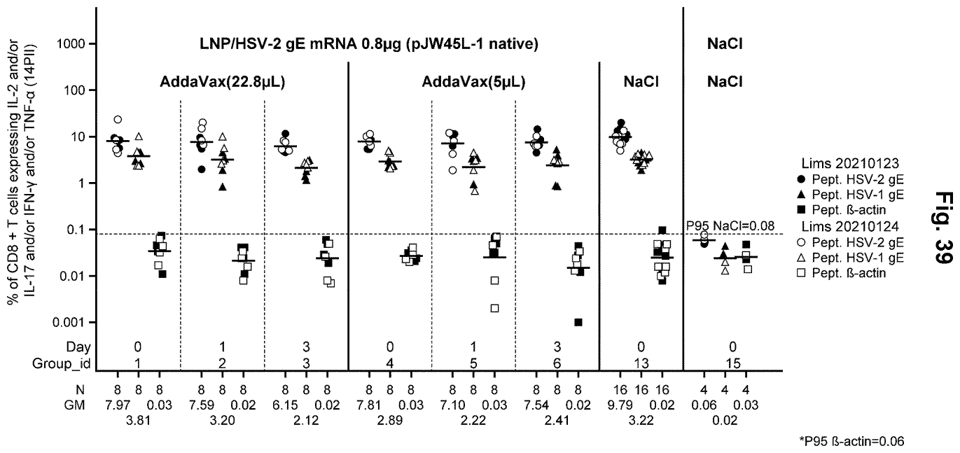

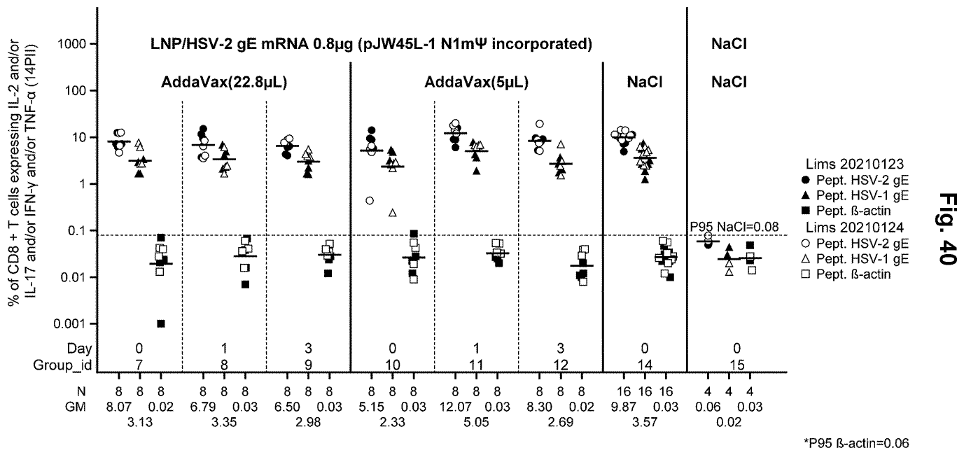

Results are provided in FIGS. 37 to 40.

### Example 6—Subject Immunisation

A subject, such as a human and in particular a human adult, is intramuscularly administered a carrier-formulated mRNA encoding an antigen, such as lipid nanoparticle (LNP)-formulated mRNA encoding a SARS-CoV-2 S protein, and a squalene emulsion adjuvant, such as a tocopherol containing squalene emulsion adjuvant.

Antibody and cellular responses to the administration are quantified.

### REFERENCES

Brito, L. A et al. A cationic nanoemulsion for the delivery of next-generation RNA vaccines, Molecular Therapy, 2014, 22, 12, 2118-2129.
Bosch et al., The Coronavirus Spike Protein Is a Class I Virus Fusion Protein: Structural and Functional Characterization of the Fusion Core Complex J. Virol. 77:8801-8811 (2003)
Carmona Martinez A, et al. A phase 1, open-label safety and immunogenicity study of an AS03-adjuvanted trivalent inactivated influenza vaccine in children aged 6 to 35 months. Hum Vaccin Immunother. 2014 10(7):1959-1968
Carter C, et al. A structure-function approach to optimizing TLR4 ligands for human vaccines, Clinical & Translation Immunology, 2016, 5, e108.
Cohet C, et al. Safety of AS03-adjuvanted Influenza Vaccines: A Review of the Evidence Vaccine 2019 37(23):3006-3021
Dayhoff M, O et al. A model of evolutionary change in proteins, Atlas of Protein Sequence and Structure, 1978 vol 5, supp. 3.
Garçon N, et al. Development and Evaluation of AS03, an Adjuvant System Containing α-Tocopherol and Squalene in an Oil-In-Water Emulsion Expert Rev Vaccines 2012 11:349-366
Fox C. Squalene Emulsions for Parenteral Vaccine and Drug Delivery Molecules 2009 14:3286-3312
Hamid, M. M. A et al. Vaccination with Plasmodium knowlesi AMA1 formulated in the novel adjuvant co-vaccine HT™ protects against blood-stage challenge in rhesus macaques. PLoS One. 2011; 6(5):e20547.
Hilgers, L. A. et al. Sucrose fatty acid sulphate esters as novel vaccine adjuvant, Vaccine, 2006, 24 Suppl 2:S2-81-2.
Jackson L, et al. Effect of Varying Doses of a Monovalent H7N9 Influenza Vaccine With and Without AS03 and MF59 Adjuvants on Immune Response: A Randomized Clinical Trial JAMA 2015 314(3):237-246
Jiang S, et al., Lipidoid-Coated Iron Oxide Nanoparticles for Efficient DNA and siRNA delivery Nano Lett., 13:1059-1064 (2013).
Kirchdoerfer et al., Stabilized coronavirus spikes are resistant to conformational changes induced by receptor recognition or proteolysis Sci. Rep. 8:15701 (2018)
Klucker M-F, et al. AF03, an Alternative Squalene Emulsion-Based Vaccine Adjuvant Prepared by a Phase Inversion Temperature Method J Pharm Sci. 2012 101(12):4490-4500
Lodaya R, et al. Formulation Design, Optimization and In Vivo Evaluations of an α-Tocopherol-Containing Self-Emulsified Adjuvant System using Inactivated Influenza Vaccine J Control Release 2019 316:12-21
Li, Structure, Function, and Evolution of Coronavirus Spike Proteins Annu. Rev. Virol. 3:237-261 (2016)
Liu et al., Improvement of Pharmacokinetic Profile of TRAIL via Trimer-Tag Enhances Its Antitumor Activity in Vivo Sci Rep. 7:8953 (2017)
Morel S, et al. Adjuvant System AS03 Containing α-Tocopherol Modulates Innate Immune Response and Leads to Improved Adaptive Immunity Vaccine 2011 29:2461-2473
O'Hagan D. MF59 Is a Safe and Potent Vaccine Adjuvant That Enhances Protection Against Influenza Virus Infection Expert Rev Vaccines 2007 6(5):699-710
O'Hagan D, et al. The History of MF59(®) Adjuvant: A Phoenix That Arose From the Ashes Expert Rev Vaccines 2013 12(1):13-30
Pallesen et al., Immunogenicity and structures of a rationally designed prefusion MERS-CoV spike antigen Proc. Natl. Acad. Sci. U.S.A. 114:E7348-E7357 (2017)
Pardi N. et al. Nature Reviews Drug Discovery 2018 17:261-279
Podda A. The Adjuvanted Influenza Vaccines With Novel Adjuvants: Experience With the MF59-adjuvanted Vaccine Vaccine 2001 19:2673-2680
Podda A and Giudice D. MF59-adjuvanted vaccines: increased immunogenicity with an optimal safety profile Expert Rev Vaccines 2003 2:197-203
Shah R, et al. The impact of size on particulate vaccine adjuvants. Nanomedicine (Lond) 2014 9:2671-2681
Shah R, et al. The development of self-emulsifying oil-in-water emulsion adjuvant and an evaluation of the impact of droplet size on performance. J Pharm Sci. 2015 104:1352-1361
Shah R, et al. The droplet size of emulsion adjuvants has significant impact on their potency, due to differences in immune cell-recruitment and -activation Scientific Reports 2019 9:11520
Sun, J et al. Analytical characterization of an oil-in-water adjuvant emulsion, AAPS PharmSciTech, 2017 18, 5, 1595-1604.
Wrapp et al., Cryo-EM structure of the 2019-nCoV spike in the prefusion conformation Science 367:1260-1263 (2020)
Younis, S et al. Down selecting adjuvanted vaccine formulations: a comparative method for harmonized evaluation, BMC Immunol, 19, 6 (2018).
Younis, S et al. Identification of adjuvants for clinical trials performed with Plasmodium falciparum AMA1 in rabbits. BMC Immunol, 20, 25 (2019).
Zhang, C et al. Advances in mRNA vaccines for infectious diseases, Front. Immunol. 2019, 10:594.
Handbook of Pharmaceutical Excipients (eds. Rowe, Sheskey, & Quinn) 6th edition, 2009 ISBN 978-0-85369-792-3
Light Scattering from Polymer Solutions and Nanoparticle Dispersions Schartl W. 2007 ISBN 978-3-540-71950-2
Vaccine Design: The Subunit and Adjuvant Approach (chapter 10; Ott G, et al.) 1995 ISBN 978-1-4613-5737-7.
Vaccine Adjuvants: Preparation Methods and Research Protocols (chapter 12; Ott G, et al.) 2000 ISBN 0-89603-735-5.
New Generation Vaccines (chapter 19) (eds. Levine et al.). 3rd edition, 2004. ISBN 0-8247-4071-8
WHO, https://www.who.int/emergencies/diseases/novel-coronavirus-2019; accessed 16 Jun. 2020.
Moderna Announces Positive Interim Phase 1 Data for its mRNA Vaccine (mRNA-1273)
Against Novel Coronavirus. Press Release May 18, 2020.
Humenza™ Summary of Product Characteristics
WO90/14837
WO2005/113782
WO2006/100109
WO2008/043774
WO2011/076807
WO2011/154443
WO2011/154444
WO2012/006359
WO2012/006372
WO2012/006376
WO2012/006378
WO2012/006380
WO2012/030901
WO2012/031043
WO2012/031046
WO2013/006825
WO2013/006834
WO2013/006837
WO2013/033563
WO2014/136086
WO2015/095340
WO2015/095346
WO2015/140138
WO2016/037053
WO2016/135154
WO2016/172396
WO2017/070620
WO2017/162265
WO2018/176103
WO2020/160080 (PCT/US2020/15565)
EP0 868 918B1
US2007/0014805
U.S. Pat. No. 4,373,071
U.S. Pat. No. 4,458,066
U.S. Pat. No. 4,500,707
U.S. Pat. No. 4,668,777
U.S. Pat. No. 4,973,679
U.S. Pat. No. 5,047,524
U.S. Pat. No. 5,132,418
U.S. Pat. No. 5,153,319
U.S. Pat. No. 5,262,530
U.S. Pat. No. 5,700,642

1. A method of eliciting an immune response in a subject, the method comprising administering to the subject (i) carrier-formulated mRNA encoding an antigen, and (ii) a squalene emulsion adjuvant.

2. A method of adjuvanting the immune response of a subject to an antigen expressed following administration of carrier-formulated mRNA encoding the antigen, the method comprising administering to the subject a squalene emulsion adjuvant.

3-6. (canceled)

7. A kit comprising:
(i) a first container comprising carrier-formulated mRNA encoding an antigen; and
(ii) a second container comprising a squalene emulsion adjuvant.

8. An immunogenic composition comprising:
(i) carrier-formulated mRNA encoding an antigen, wherein the carrier is an LNP, and
(ii) a squalene emulsion adjuvant.

9. (canceled)

10. The method of claim 1, wherein the squalene emulsion adjuvant has an average droplet size of less than 1 um.

11. The method of claim 10, wherein the squalene emulsion adjuvant has an average droplet size of 50 to 200 nm.

12. The method of claim 1, wherein the squalene emulsion adjuvant has a polydispersity of 0.5 or less.

13. The method of claim 1, wherein the squalene emulsion adjuvant comprises a surfactant that comprises one or more of from poloxamer 401, poloxamer 188, polysorbate 80, sorbitan trioleate, sorbitan monooleate and polyoxyethylene 12 cetyl/stearyl ether.

14. The method according to of claim 1, wherein the squalene emulsion adjuvant comprises a surfactant that comprises polysorbate 80.

15. The method of claim 1, wherein the amount of squalene in a single dose of the squalene emulsion adjuvant is 20 mg or less.

16. The method of claim 1, wherein the amount of squalene in a single dose of the squalene emulsion adjuvant is 0.5 mg or more.

17. The method of claim 1, wherein the weight ratio of squalene to surfactant in the squalene emulsion adjuvant is 0.73 to 6.6.

18. The method of claim 1, wherein the squalene emulsion adjuvant consists essentially of squalene, surfactant and water.

19. The method of claim 1, wherein the squalene emulsion adjuvant comprises squalene, polysorbate 80, sorbitan trioleate and water.

20. The method of claim 1, wherein the squalene emulsion adjuvant comprises squalene, sorbitan monooleate, polyoxyethylene cetostearyl ether, water, and mannitol.

21. The method of claim 1, wherein the squalene emulsion adjuvant comprises squalene, phosphatidyl choline, poloxamer 188, water, and glycerol.

22. The method, of claim 1, wherein the squalene emulsion adjuvant comprises tocopherol.

23. The method of claim 22, wherein the squalene emulsion adjuvant consists essentially of squalene, tocopherol, surfactant and water.

24. The method of claim 22, wherein the weight ratio of squalene to tocopherol in the squalene emulsion adjuvant is 20 or less.

25. The method of claim 24, wherein the weight ratio of squalene to tocopherol in the squalene emulsion adjuvant is 0.1 or more.

26. The method of claim 25, wherein the squalene emulsion adjuvant comprises squalene, tocopherol, polysorbate 80 and water.

27. The method of claim 26, wherein the squalene emulsion adjuvant consists essentially of squalene, tocopherol, polysorbate 80 and water.

28. The method of claim 17, wherein the squalene emulsion adjuvant comprises squalene, tocopherol, phosphatidyl choline, poloxamer 188, water, and glycerol.

29. The method of claim 1, wherein the subject is human.

30-35. (canceled)

36. The method of claim 1, wherein the mRNA comprises at least one chemical modification.

37-38. (canceled)

39. The method of claim 36, wherein the chemical modification is N1-methylpseudouridine.

40. (canceled)

41. The method of claim 1, wherein the mRNA is not chemically modified.

42. The method of claim 1, wherein each mRNA is non-replicating.

43. (canceled)

44. The method of claim 1, wherein the mRNA is self-replicating.

45. (canceled)

47. The method of claim 1, wherein a single dose of mRNA is 0.001 to 1000 ug.

48. The method of claim 1, wherein the carrier is a lipid nanoparticle (LNP).

49-52. (canceled)

53. The method of claim 48, wherein the LNP comprise a PEG-modified lipid at around 0.5 to 15 molar %, a non-cationic lipid at around 5 to 25 molar %, a sterol at around 25 to 55 molar % and an ionisable cationic lipid at around 20 to 60 molar %.

54-56. (canceled)

57. The method of claim 48, wherein at least 85% of the RNA is encapsulated in the LNP.

58. The method of claim 1, wherein the carrier-formulated mRNA and squalene emulsion adjuvant are administered as a co-formulation.

59. The method of claim 1, wherein the carrier-formulated mRNA and squalene emulsion adjuvant are administered as separate formulations.

60. The method of claim 59, wherein the carrier-formulated mRNA and squalene emulsion adjuvant are administered within 24 hours of each other.

61. (canceled)

62. The method of claim 61, wherein the carrier-formulated mRNA and squalene emulsion adjuvant are administered to the same location.

63. A process for manufacturing the composition of claim 8, comprising: a step of combining (i) a carrier formulated mRNA, wherein the carrier is LNP, with (ii) a squalene emulsion adjuvant.


SEQUENCE LISTING (13 sequences)

SEQ ID NO: 1 - protein, 1273 aa, Human coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1208 aa, Human coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 3 - protein, 192 aa, Human coronavirus
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 4 - protein, 1208 aa, Artificial Sequence
  SARS-CoV-2 Stabilized Spike protein ectodomain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 5 - DNA, 2864 bp, Artificial Sequence
  Polynucleotide sequence of HSV-2 gE/gI insert
gggcccctataactctctacggctaacctgaatggactacgacatagtctagtccgccaagatggccaggggagccggcctcgtgttcttcgtgggcgtgtgggtggtgagctgtctggccgctgcccctagaaccagctggaagagagtgacctccggagaggacgtggtgctgctccctgctcccgctggacctgaggaaagaaccagagcccacaagctgctgtgggctgctgagcctctggacgcctgcggacctctgagaccttcctgggtggctctgtggcctcccagaagagtgctggagacagtggtggacgccgcctgcatgagagcccctgaacccctggccatcgcctactcccccccttttcccgccggagacgagggcctgtatagcgagctggcctggagagacagggtggccgtggtcaatgagagcctggtcatctacggagccctggagaccgacagcggcctgtataccctgagcgtggtgggcctgagcgacgaagccagacaggtggcttccgtcgtgctggtggtggaacctgcccccgtccctacacctacccctgacgactacgacgaggaggacgacgccggcgtgtccgaaagaacccccgtgagcgtgcctccccccacccctcctagaagacctcctgtggctcctcctacccaccccagggtgatccctgaggtgagccacgtcagaggcgtgaccgtgcacatggagacacctgaggccatcctgttcgcccctggcgagaccttcggaaccaatgtgtccatccacgccattgcccacgacgacggcccctacgccatggacgtggtgtggatgaggtttgacgtgccctccagctgcgccgagatgaggatctacgaggcctgcctgtaccacccccagctgcccgagtgtctgtcccctgctgacgctccttgcgccgtcagcagctgggcctatagactggccgtgagatcctacgctggctgtagcagaaccaccaggccccccagatgcttcgccgaagccagaatggagcctgtgcctggactggcctggctggcttccaccgtgaatctggagtttcagcacgccagcccccaacacgccggactgtacctgtgcgtggtgtacgtggacgaccacatccatgcctggggacacatgaccatcagcaccgccgcccagtacaggaacgctgtggtggaacagcacctgccccagaggcaacctgagcccgtggagcccaccagacctcatgtgagagcccctccccctgctccctccgccagaggacctctgagactgggctgattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagtctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatgcctggcaggagcctgcaaggcctggccatcctgggcctgtgggtctgtgctaccggcctggtggtgagaggacccaccgtgtccctggtgagcgacagcctggtggatgccggagctgtgggacctcagggcttcgtggaagaggacctgagggtgttcggcgaactccactttgtgggcgcccaggtgccccacaccaactactacgacggcatcatcgagctgttccactaccccctgggtaatcattgtcccagagtggtgcacgtggtgaccctgaccgcctgtcctagaaggcccgctgtggccttcaccctgtgcagaagcacccaccacgcccacagccctgcctaccccacactggaactgggactggccagacagcccctgctgagggtgaggacagccaccagggactatgccggactgtacgtgctgagagtgtgggtgggaagcgccaccaacgcctccctgttcgtgctgggcgtggctctgagcgccaatggcaccttcgtgtacaacggctccgactatggcagctgtgaccccgcccagctcccctttagcgctcctaggctgggacctagctccgtgtacacccccggagccagcagacccacaccccccagaaccaccaccagccctagctcccctcgtgatcctacccctgcccctggcgatacaggaacccctgcccccgctagcggagaaagagcccctcccaacagcaccaggtccgccagcgagtccaggcacaggctgacatgatgaggcgcgcccacccagcggccgc

SEQ ID NO: 6 - DNA, 12775 bp, Artificial Sequence
  Polynucleotide sequence of parent SAM pTC83R_ P989 plasmid
aagttgggtaacgccagggttttcccagtcacacgcgtaatacgactcactatagataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggccaggggagccggcctcgtgttcttcgtgggcgtgtgggtggtgagctgtctggccgctgcccctagaaccagctggaagagagtgacctccggagaggacgtggtgctgctccctgctcccgctggacctgaggaaagaaccagagcccacaagctgctgtgggctgctgagcctctggacgcctgcggacctctgagaccttcctgggtggctctgtggcctcccagaagagtgctggagacagtggtggacgccgcctgcatgagagcccctgaacccctggccatcgcctactcccccccttttcccgccggagacgagggcctgtatagcgagctggcctggagagacagggtggccgtggtcaatgagagcctggtcatctacggagccctggagaccgacagcggcctgtataccctgagcgtggtgggcctgagcgacgaagccagacaggtggcttccgtcgtgctggtggtggaacctgcccccgtccctacacctacccctgacgactacgacgaggaggacgacgccggcgtgtccgaaagaacccccgtgagcgtgcctccccccacccctcctagaagacctcctgtggctcctcctacccaccccagggtgatccctgaggtgagccacgtcagaggcgtgaccgtgcacatggagacacctgaggccatcctgttcgcccctggcgagaccttcggaaccaatgtgtccatccacgccattgcccacgacgacggcccctacgccatggacgtggtgtggatgaggtttgacgtgccctccagctgcgccgagatgaggatctacgaggcctgcctgtaccacccccagctgcccgagtgtctgtcccctgctgacgctccttgcgccgtcagcagctgggcctatagactggccgtgagatcctacgctggctgtagcagaaccaccaggccccccagatgcttcgccgaagccagaatggagcctgtgcctggactggcctggctggcttccaccgtgaatctggagtttcagcacgccagcccccaacacgccggactgtacctgtgcgtggtgtacgtggacgaccacatccatgcctggggacacatgaccatcagcaccgccgcccagtacaggaacgctgtggtggaacagcacctgccccagaggcaacctgagcccgtggagcccaccagacctcatgtgagagcccctccccctgctccctccgccagaggacctctgagactgggctgattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagtctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatgcctggcaggagcctgcaaggcctggccatcctgggcctgtgggtctgtgctaccggcctggtggtgagaggacccaccgtgtccctggtgagcgacagcctggtggatgccggagctgtgggacctcagggcttcgtggaagaggacctgagggtgttcggcgaactccactttgtgggcgcccaggtgccccacaccaactactacgacggcatcatcgagctgttccactaccccctgggtaatcattgtcccagagtggtgcacgtggtgaccctgaccgcctgtcctagaaggcccgctgtggccttcaccctgtgcagaagcacccaccacgcccacagccctgcctaccccacactggaactgggactggccagacagcccctgctgagggtgaggacagccaccagggactatgccggactgtacgtgctgagagtgtgggtgggaagcgccaccaacgcctccctgttcgtgctgggcgtggctctgagcgccaatggcaccttcgtgtacaacggctccgactatggcagctgtgaccccgcccagctcccctttagcgctcctaggctgggacctagctccgtgtacacccccggagccagcagacccacaccccccagaaccaccaccagccctagctcccctcgtgatcctacccctgcccctggcgatacaggaacccctgcccccgctagcggagaaagagcccctcccaacagcaccaggtccgccagcgagtccaggcacaggctgacatgatgaggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgatt

SEQ ID NO: 7 - DNA, 10522 bp, Artificial Sequence
  Polynucleotide sequence of SAM gE_P317R_IRES_gI transcript
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggccaggggagccggcctcgtgttcttcgtgggcgtgtgggtggtgagctgtctggccgctgcccctagaaccagctggaagagagtgacctccggagaggacgtggtgctgctccctgctcccgctggacctgaggaaagaaccagagcccacaagctgctgtgggctgctgagcctctggacgcctgcggacctctgagaccttcctgggtggctctgtggcctcccagaagagtgctggagacagtggtggacgccgcctgcatgagagcccctgaacccctggccatcgcctactcccccccttttcccgccggagacgagggcctgtatagcgagctggcctggagagacagggtggccgtggtcaatgagagcctggtcatctacggagccctggagaccgacagcggcctgtataccctgagcgtggtgggcctgagcgacgaagccagacaggtggcttccgtcgtgctggtggtggaacctgcccccgtccctacacctacccctgacgactacgacgaggaggacgacgccggcgtgtccgaaagaacccccgtgagcgtgcctccccccacccctcctagaagacctcctgtggctcctcctacccaccccagggtgatccctgaggtgagccacgtcagaggcgtgaccgtgcacatggagacacctgaggccatcctgttcgcccctggcgagaccttcggaaccaatgtgtccatccacgccattgcccacgacgacggcccctacgccatggacgtggtgtggatgaggtttgacgtgccctccagctgcgccgagatgaggatctacgaggcctgcctgtaccacccccagctgcccgagtgtctgtcccctgctgacgctccttgcgccgtcagcagctgggcctatagactggccgtgagatcctacgctggctgtagcagaaccaccaggccccccagatgcttcgccgaagccagaatggagcctgtgcctggactggcctggctggcttccaccgtgaatctggagtttcagcacgccagcccccaacacgccggactgtacctgtgcgtggtgtacgtggacgaccacatccatgcctggggacacatgaccatcagcaccgccgcccagtacaggaacgctgtggtggaacagcacctgccccagaggcaacctgagcccgtggagcccaccagacctcatgtgagagcccctccccctgctccctccgccagaggacctctgagactgggctgattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagtctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatgcctggcaggagcctgcaaggcctggccatcctgggcctgtgggtctgtgctaccggcctggtggtgagaggacccaccgtgtccctggtgagcgacagcctggtggatgccggagctgtgggacctcagggcttcgtggaagaggacctgagggtgttcggcgaactccactttgtgggcgcccaggtgccccacaccaactactacgacggcatcatcgagctgttccactaccccctgggtaatcattgtcccagagtggtgcacgtggtgaccctgaccgcctgtcctagaaggcccgctgtggccttcaccctgtgcagaagcacccaccacgcccacagccctgcctaccccacactggaactgggactggccagacagcccctgctgagggtgaggacagccaccagggactatgccggactgtacgtgctgagagtgtgggtgggaagcgccaccaacgcctccctgttcgtgctgggcgtggctctgagcgccaatggcaccttcgtgtacaacggctccgactatggcagctgtgaccccgcccagctcccctttagcgctcctaggctgggacctagctccgtgtacacccccggagccagcagacccacaccccccagaaccaccaccagccctagctcccctcgtgatcctacccctgcccctggcgatacaggaacccctgcccccgctagcggagaaagagcccctcccaacagcaccaggtccgccagcgagtccaggcacaggctgacatgatgaggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaa

SEQ ID NO: 8 - RNA, 10522 nt, Artificial Sequence
  SAM pTC83R_P989 transcript theoretical RNA sequence
gauaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagauggccaggggagccggccucguguucuucgugggcguguggguggugagcugucuggccgcugccccuagaaccagcuggaagagagugaccuccggagaggacguggugcugcucccugcucccgcuggaccugaggaaagaaccagagcccacaagcugcugugggcugcugagccucuggacgccugcggaccucugagaccuuccuggguggcucuguggccucccagaagagugcuggagacagugguggacgccgccugcaugagagccccugaaccccuggccaucgccuacucccccccuuuucccgccggagacgagggccuguauagcgagcuggccuggagagacaggguggccguggucaaugagagccuggucaucuacggagcccuggagaccgacagcggccuguauacccugagcguggugggccugagcgacgaagccagacagguggcuuccgucgugcuggugguggaaccugcccccgucccuacaccuaccccugacgacuacgacgaggaggacgacgccggcguguccgaaagaacccccgugagcgugccuccccccaccccuccuagaagaccuccuguggcuccuccuacccaccccagggugaucccugaggugagccacgucagaggcgugaccgugcacauggagacaccugaggccauccuguucgccccuggcgagaccuucggaaccaauguguccauccacgccauugcccacgacgacggccccuacgccauggacgugguguggaugagguuugacgugcccuccagcugcgccgagaugaggaucuacgaggccugccuguaccacccccagcugcccgagugucuguccccugcugacgcuccuugcgccgucagcagcugggccuauagacuggccgugagauccuacgcuggcuguagcagaaccaccaggccccccagaugcuucgccgaagccagaauggagccugugccuggacuggccuggcuggcuuccaccgugaaucuggaguuucagcacgccagcccccaacacgccggacuguaccugugcgugguguacguggacgaccacauccaugccuggggacacaugaccaucagcaccgccgcccaguacaggaacgcugugguggaacagcaccugccccagaggcaaccugagcccguggagcccaccagaccucaugugagagccccucccccugcucccuccgccagaggaccucugagacugggcugauuaaaacagcuguggguuguucccacccacagggcccacugggcgcuagcacucugauuuuacgaaauccuugugcgccuguuuuauaucccuucccuaauucgaaacguagaagcaaugcgcaccacugaucaauaguaggcguaacgcgccaguuacgucaugaucaagcauaucuguucccccggacugaguaucaauagacugcuuacgcgguugaaggagaaaacguucguuauccggcuaacuacuucgagaagcccaguaacaccauggaagcugcaggguguuucgcucagcacuucccccguguagaucaggucgaugagccacugcaauccccacaggugacuguggcaguggcugcguuggcggccugccuauggggagacccauaggacgcucuaauguggacauggugcgaagagucuauugagcuaguuaguaguccuccggccccugaaugcggcuaauccuaacugcggagcacaugccuucaacccagaggguagugugucguaacgggcaacucugcagcggaaccgacuacuuuggguguccguguuucuuuuuuauucuuauauuggcugcuuauggugacaauuacagaauuguuaccauauagcuauuggauuggccauccgguguguaauagagcuguuauauaccuauuuguuggcuuuguaccacuaacuuuaaaaucuauaacuacccucaacuuuauauuaacccucaauacaguugaccaugccuggcaggagccugcaaggccuggccauccugggccugugggucugugcuaccggccugguggugagaggacccaccgugucccuggugagcgacagccugguggaugccggagcugugggaccucagggcuucguggaagaggaccugaggguguucggcgaacuccacuuugugggcgcccaggugccccacaccaacuacuacgacggcaucaucgagcuguuccacuacccccuggguaaucauugucccagaguggugcacguggugacccugaccgccuguccuagaaggcccgcuguggccuucacccugugcagaagcacccaccacgcccacagcccugccuaccccacacuggaacugggacuggccagacagccccugcugagggugaggacagccaccagggacuaugccggacuguacgugcugagagugugggugggaagcgccaccaacgccucccuguucgugcugggcguggcucugagcgccaauggcaccuucguguacaacggcuccgacuauggcagcugugaccccgcccagcuccccuuuagcgcuccuaggcugggaccuagcuccguguacacccccggagccagcagacccacaccccccagaaccaccaccagcccuagcuccccucgugauccuaccccugccccuggcgauacaggaaccccugcccccgcuagcggagaaagagccccucccaacagcaccagguccgccagcgaguccaggcacaggcugacaugaugaggcgcgcccacccagcggccgcauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaaa

SEQ ID NO: 9 - protein, 419 aa, Artificial Sequence
  Polypeptide sequence of HSV-2 gE P317R
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTRPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLG

SEQ ID NO: 10 - protein, 256 aa, Artificial Sequence
  Polypeptide sequence of HSV-2 gI
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTSPSSPRDPTPAPGDTGTPAPASGERAPPNSTRSASESRHRLT

SEQ ID NO: 11 - RNA, 1593 nt, Artificial Sequence
  gE mRNA with 5′ UTR, 3′ UTR and polyA tail
gggaaauaagagagaaaagaagaguaagaagaaauauaagaccccggcgccgccaccauggccaggggagccggccucguguucuucgugggcguguggguggugagcugucuggccgcugccccuagaaccagcuggaagagagugaccuccggagaggacguggugcugcucccugcucccgcuggaccugaggaaagaaccagagcccacaagcugcugugggcugcugagccucuggacgccugcggaccucugagaccuuccuggguggcucuguggccucccagaagagugcuggagacagugguggacgccgccugcaugagagccccugaaccccuggccaucgccuacucccccccuuuucccgccggagacgagggccuguauagcgagcuggccuggagagacaggguggccguggucaaugagagccuggucaucuacggagcccuggagaccgacagcggccuguauacccugagcguggugggccugagcgacgaagccagacagguggcuuccgucgugcuggugguggaaccugcccccgucccuacaccuaccccugacgacuacgacgaggaggacgacgccggcguguccgaaagaacccccgugagcgugccuccccccaccccuccuagaagaccuccuguggcuccuccuacccaccccagggugaucccugaggugagccacgucagaggcgugaccgugcacauggagacaccugaggccauccuguucgccccuggcgagaccuucggaaccaauguguccauccacgccauugcccacgacgacggccccuacgccauggacgugguguggaugagguuugacgugcccuccagcugcgccgagaugaggaucuacgaggccugccuguaccacccccagcugcccgagugucuguccccugcugacgcuccuugcgccgucagcagcugggccuauagacuggccgugagauccuacgcuggcuguagcagaaccaccccuccccccagaugcuucgccgaagccagaauggagccugugccuggacuggccuggcuggcuuccaccgugaaucuggaguuucagcacgccagcccccaacacgccggacuguaccugugcgugguguacguggacgaccacauccaugccuggggacacaugaccaucagcaccgccgcccaguacaggaacgcugugguggaacagcaccugccccagaggcaaccugagcccguggagcccaccagaccucaugugagagccccucccccugcucccuccgccagaggaccucugagacugggcugaucuagacggcgcgcccacccagcggccgcugauaauaggcuggagccucgguggccaugcuucuugccccuugggccuccccccagccccuccuccccuuccugcacccguacccccguggucuuugaauaaagucugauuuucauugcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - RNA, 1593 nt, Artificial Sequence; identical to SEQ ID NO: 11
  gE mRNA with 5′ UTR, 3′ UTR and polyA tail - all U replaced with N1methyl-pseudouridine

SEQ ID NO: 13 - protein, 419 aa, Artificial Sequence
  HSV-2 gE
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLG